(19) United States
(12) Patent Application Publication
(10) Pub. No.: US 2026/0263685 A1
(43) Pub. Date: Sep. 10, 2026

(54) A cassette of a medicament delivery device and a medicament delivery device

(71) Applicant: SHL Medical AG, Zug (CH)

(72) Inventors: Hsuan Wang, Zug (CH); Torbjörn Neby, Nacka Strand (SE); Thomas Daniel James, Baltimore, MD (US); Sebastian Karlsson, Stigtomta (SE); Stephen Allison Porter, Baltimore, MD (US); Mariano Mumpower, Baltimore, MD (US); Ralph Howald, Zug (CH); Christopher James Franzese, Boonton, NJ (US); Chester Boyd Larrow, Baltimore, MD (US); Yukiko Naoi, Norwalk, CT (US); Daniel Scott, Deerfield Beach, FL (US); Christoph Egloff, Zug (CH); Jacob Leon Turetsky, Norwalk, CT (US); Jeffrey Kapec, Norwalk, CT (US); Plamen Balkandjiev, Nacka Strand (SE); Florence Olivia Stevenson, Baltimore, MD (US); Joshua Thomas Hartl, Baltimore, MD (US); Benjamin Richard Lane, Baltimore, MD (US); Katherine Ford Brigham, Boonton, NJ (US); Amy Catherine Rinaldi, Boonton, NJ (US); James Thomas Hawthorne, Boonton, NJ (US); Demosthenis Katsaros, Boonton, NJ (US); Mattias Myrman, Nacka Strand (SE); Ola Hallström, Nacka Strand (SE); Rasmus Renstad, Nacka Strand (SE); Nils Weber, Zug (CH); Samuel Wyler, Zug (CH); Lukas Bannwart, Zug (CH); Nuria De Santiago Giner, Zug (CH); Christopher Martin Defrancisci, Baltimore, MD (US); Jeffrey Manfred Gunnarsson, Baltimore, MD (US); Mason Mitchell Watts, Baltimore, MD (US); Raghuraj Jitendrasinh Chauhan, Baltimore, MD (US); Christopher Fleck, Baltimore, MD (US); Martin Michael Coyne, III, Boonton, NJ (US)

(21) Appl. No.: 18/871,915

(22) PCT Filed: Jun. 9, 2023
(86) PCT No.: PCT/EP2023/065479

§ 371 Date: Dec. 5, 2024

Related U.S. Application Data
(60) Provisional application No. 63/350,463, filed on Jun. 9, 2022.
(60) Provisional application No. 63/392,539, filed on Jul. 27, 2022.
(60) Provisional application No. 63/425,713, filed on Nov. 16, 2022.
(60) Provisional application No. 63/464,033, filed on May 4, 2023.

(30) Foreign Application Priority Data
Sep. 30, 2022 (EP) 22199278.7
Jun. 7, 2023 (EP) 23386042.8

(51) Int. Cl. A61M 5/14; A61J 1/10; A61M 5/142; A61M 5/162; A61M 5/168

(52) U.S. Cl. CPC A61M 5/1413; A61M 5/142; A61M 5/162; A61M 5/16881; A61J 1/10; A61M 2205/0294; A61M 2205/123

(57) ABSTRACT

The present disclosure provides a cassette of a medicament delivery device, wherein the medicament delivery device comprises a fluid pressure power source. The cassette includes a container carrier, and a medicament container with a body and a fluid outlet. The medicament container is at least partially arranged within the container carrier. The container carrier comprises a fluid-tight chamber including an inlet. The inlet is configured to fluidly connect to an outlet of the fluid pressure power source of the reusable body of the medicament delivery device.

### CROSS-REFERENCE TO RELATED APPLICATIONS

The present application is a U.S. National Phase Application pursuant to 35 U.S.C. § 371 of International Application No. PCT/EP2023/065479 filed Jun. 9, 2023, which claims priority to (i) U.S. Provisional Application No. 63/350,463 filed Jul. 27, 2022, (ii) U.S. Provisional Application No. 63/392,539 filed Jun. 9, 2022, (iii) EP Patent Application No. 22199278.7 filed Sep. 30, 2022, (iv) U.S. Provisional Application No. 63/425,713 filed Nov. 16, 2022, (v) U.S. Provisional Application No. 63/464,033 filed May 4, 2023, and (vi) EP Patent Application No. 23386042.8 filed Jun. 7, 2023. The entire disclosure contents of these applications are herewith incorporated by reference into the present application.

### TECHNICAL FIELD

The present disclosure generally relates to a cassette of a medicament delivery device, and particularly to a cassette with a medicament container comprising a flexible portion and the flexible portion is coupled to a fluid-tight chamber.

### BACKGROUND

Medicament delivery devices such as pen-type manual injectors or auto-injectors are generally known for the self-administration of a medicament by patients without formal medical training. For administrating high volume and/or viscosity medicament, or multiple different medicaments, a motor-driven medicament delivery device is desirable.

Many motor-driven systems exist to deliver medicaments, as in the case of parenteral delivery. For instance, peristaltic drives or piston pumps are common in the art. However, motor-driven medicament delivery systems have disadvantages in some applications. These disadvantages can become pronounced when considering a device that can deliver multiple medicaments, as in the case of a device that is configured to administer sequential medication regimens without the in-process intervention of a healthcare professional (HCP). Additionally, in-line pumping elements often include silicone oil as means of lubrication, which while largely inert, can introduce potential drug stability considerations when particularly sensitive medicaments are delivered through them.

It is difficult to accommodate multiple medications without increasing the size of the drive unit; if a device requires a separate motor for each additional medication, the power requirement, size, complexity, and cost of the device in each application becomes potentially infeasible to manage, especially during final configuration and assembly of the device (i.e., dispensing) by a pharmacist. Administration of multiple medications requires a motor to be driven for a sustained period, for example between 1-6 hours. The required motor may consume large amounts of power, necessitating larger batteries or permanent power sources, which add weight or reduce mobility of a patient using the device. Further, to provide the needed torque or motive forces, powerful motors or large gear trains may be required, adding weight and noise which may be disconcerting for patients. Additionally, such components may add manufacturing complexity and attendant cost which are undesirable in a device, as in the case of devices used at home. Thus, there is a need for a lightweight delivery device whose drive is preferably light, quiet, and powerful, preferably external to the medicament flow path, and preferably independent of the number of medications delivered or the duration of delivery.

In some cases, multiple medications must remain fluidically separate to prevent mixing of medications and potential compatibility issues between medications and/or medication formulation components (i.e., excipients) that may cause unintended or undesirable effects (e.g., loss of potency, efficacy, or aggregation). While a single medication device need not be concerned with this, this no longer holds true when the drug delivery device is intended to deliver two or more drug products.

Different medications used with the system may each have different volumes and/or viscosities. For instance, some medications may be fixed doses, while others may be variable doses that are patient-specific. The power requirement for a motor-driven pump may vary based on the desired flow rate and volume to be delivered for each medication. This may require novel approaches to program the pump for each medication. This complicates dispensing. Thus, there is a need for improved systems and apparatus for administration that may deliver a volume of medication at a given viscosity that is not known a priori using separate single-use fluidic paths (e.g., reservoir and tubing set). Further, there is a need for improved systems and apparatus for administration of multiple medications while using a single pump mechanism that does not require a priori knowledge of the number, volume, or viscosity of medications, and may be flexibly configured to deliver one or more medications in a desired sequence governed by a prescribed medication regimen.

### SUMMARY

The invention is defined by the appended claims, to which reference should now be made.

There is hence provided a cassette of a medicament delivery device, wherein the medicament delivery device comprises a reusable body comprising a fluid pressure power source. The cassette comprises a container carrier, and a medicament container with a body and a fluid outlet. The body comprises a flexible portion. The medicament container is at least partially arranged within the container carrier. The container carrier comprises a fluid-tight chamber. The fluid-tight chamber comprises an inlet. The inlet is configured to fluidly connect to an outlet of the fluid pressure power source of the reusable body of the medicament delivery device. The fluid-tight chamber is coupled to the flexible portion of the body of the medicament container so that when the inlet receives output fluid from the fluid pressure power source, the output fluid flows into the fluid-tight chamber and the medicament contained within the medicament container is pressed out under the pressure of the output fluid. The fluid outlet is configured to be connected to a medicament delivery member of the medicament delivery device when the cassette is attached to the medicament delivery device.

The cassette is configured for use with a medicament delivery device.

Preferably, according to another embodiment, the cassette is configured to be used with a portable medicament delivery device.

Preferably, according to another embodiment, the body of the medicament container comprises a flexible bag and/or a flexible tube.

Preferably, according to another embodiment, the body of the medicament container is a flexible bag received within the fluid-tight chamber.

Preferably, according to another embodiment, the fluid pressure power source is a pneumatic power source or a hydraulic power source. The pneumatic power source is configured to output gas to the cassette and expel the medicament contained within the cassette by increasing the gas pressure around the fluid-tight chamber. The hydraulic power source is configured to output liquid to the cassette and expel the medicament contained within the cassette by increasing the liquid pressure around the fluid-tight chamber. When the fluid pressure power source is the pneumatic power source, the fluid-tight chamber is gas-tight. When the fluid pressure power source is hydraulic power source, the fluid-tight chamber can be either gas-tight or liquid-tight.

Therefore, a medicament delivery device comprising the cassette as disclosed in this disclosure can provide a drive system for a pneumatic pressure-driven or hydraulic pressure-driven medicament delivery device capable of delivering one or more medicaments in a desired sequence, each at a desired flow rate of delivery of the medicament. The system does not require prior knowledge of the volume and/or viscosity of each medicament. Therefore, the reusable body of the medicament delivery device can be used for different medicaments contained within different disposable cassettes. The medicament container can be filled with medication by a pharmacy (“fill at point of use”), e.g., in a hospital or infusion center compounding suite) or by a drug maker (“prefilled”). During use, fluid, e.g., gas or liquid, can be added to the interior of the cassette at a known mass flow rate to collapse the flexible container inside the cassette and deliver its contents to a patient at a known and controllable volumetric flow rate.

Preferably, according to another embodiment, the cassette is configured to be attached to the reusable body of the medicament delivery device.

Preferably, according to another embodiment, the cassette comprises a cassette housing.

Preferably, according to another embodiment, the cassette housing is configured to be attached to the reusable body of the medicament delivery device.

Preferably, according to another embodiment, the container carrier is arranged within the cassette housing.

Alternatively, the container carrier is configured to be operably attached to the reusable body of the medicament delivery device. In this example, the cassette does not need the cassette housing.

Preferably, according to another embodiment, the cassette is configured to be releasably attached to the reusable body of the medicament delivery device.

Preferably, according to another embodiment, the fluid-tight chamber is a secondary flexible bag that fully encloses the medicament container. This could be accomplished via a multiple-layer bag assembly with one port as the fluid outlet and the other port as the inlet of the fluid-tight chamber.

Preferably, according to another embodiment, the body of the medicament container comprises a delivery tube.

Preferably, according to another embodiment, the delivery tube is the flexible tube of the body of the medicament container.

Preferably, according to another embodiment, the flexible portion of the body is at least partially accommodated within the fluid-tight chamber.

Preferably, according to another embodiment, the fluid-tight chamber comprises a tube inlet and a tube outlet. The flexible delivery tube is configured to be positioned between the tube inlet and the tube outlet.

Preferably, according to another embodiment, the flexible delivery tube comprises two tube valves. The fluid-tight chamber is configured to surround a portion of the flexible delivery tube between the two tube valves.

Preferably, according to another embodiment, the fluid-tight chamber comprises an outlet configured to be connected to a vacuum device, so that when the pressure within the fluid-tight chamber is reduced, medicament contained within the medicament container is sucked into the flexible delivery tube.

Preferably, according to another embodiment, the inlet of the fluid-tight chamber is the outlet of the fluid-tight chamber.

Alternatively, according to another embodiment, the medicament container is configured to be attached to a tube set comprising the delivery tube.

Preferably, according to another embodiment, the fluid outlet of the medicament container is configured to be connected to a medicament delivery member via the delivery tube. In this example, the fluid outlet of the medicament container is configured to be attached at one end of the delivery tube; and the medicament delivery member is configured to be attached at the other end of the delivery tube.

Preferably, according to another embodiment, the tube set comprises a piercing member configured to penetrate the fluid outlet of the medicament container to establish fluid communication between the delivery tube and the medicament container.

Preferably, according to another embodiment, the piercing member is a needle cannular.

Preferably, according to another embodiment, the delivery tube comprises a tube valve.

Preferably, according to another embodiment, the tube valve is an in-line valve included in the delivery tube.

Preferably, according to another embodiment, the tube valve of the delivery tube is a one-way valve so that the fluid cannot flow towards the medicament container through the delivery tube.

Preferably, according to another embodiment, the tube valve of the delivery tube is an umbrella valve or a Belleville valve or a ball valve or a pinch valve.

Preferably, according to another embodiment, the medicament delivery device comprises a delivery flow rate sensor.

Preferably, according to another embodiment, the delivery flow rate sensor is attached at the fluid outlet of the medicament container and/or the delivery tube of the tube set.

Preferably, according to another embodiment, the container carrier comprises a container chamber configured to at least partially accommodate the medicament container.

Preferably, according to another embodiment, the fluid-tight chamber is inflatable. The fluid-tight chamber is adjacent to the flexible portion of the body so that the output fluid from the fluid pressure power source is configured to flow into the fluid-tight chamber and inflate the fluid-tight chamber to press on the medicament container.

Preferably, according to another embodiment, the inlet of the fluid-tight chamber comprises a valve.

Preferably, according to another embodiment, the valve of the inlet of the fluid-tight chamber is a one-way valve so that the fluid can only pass the one-way valve for entering the fluid-tight chamber.

Preferably, according to another embodiment, the cassette comprises at least two medicament containers.

Preferably, according to another embodiment, the cassette comprises at least two container carriers.

Preferably, according to another embodiment, the at least two container carriers the at least two medicament containers respectively.

Preferably, according to another embodiment, the medicament delivery device comprises a multi-directions valve, e.g., 2/2-way valve, 3/2-way valve, 5/2-way valve, connected to the fluid pressure power source. One port of the multi-directions valve is configured to be attached to the fluid-tight chamber in one of the least two container carriers, and another port of the multi-directions valve is configured to be attached to the fluid-tight chamber in another one of the least two container carriers.

Preferably, according to another embodiment, the cassette comprises one container carrier; and the container carrier comprises at least two fluid-tight chambers.

Preferably, according to another embodiment, the at least two fluid-tight chambers container carriers accommodate the at least two medicament containers respectively.

Preferably, according to another embodiment, each medicament container is provided with a fluidically separate connection to the patient in a manner by which separate medications do not mix during administration unless desired at the patient site.

Preferably, according to another embodiment, a one-way valve is arranged between the at least two fluid-tight chambers.

Preferably, according to another embodiment, the one-way valve arranged between the at least two fluid-tight chambers is a single-use valve, e.g., a frangible valve.

Preferably, according to another embodiment, only one fluid-tight chamber of one of the at least two container carriers comprises the inlet that is configured to be fluidly connected to the fluid pressure power source.

Preferably, according to another embodiment, the one-way valve between the two fluid-tight chambers is configured to open when a first fluid pressure threshold is reached. The valve of the inlet of the fluid-tight chamber that is configured to be fluidly connected to the fluid pressure power source is configured to open when a second fluid pressure threshold is reached. The first fluid pressure threshold is equal to or greater than the second fluid pressure threshold.

Preferably, according to another embodiment, the valve of the inlet of the fluid-tight chamber that is configured to be fluidly connected to the fluid pressure power source is configured to open when a second fluid pressure threshold is reached. The predetermined threshold is greater than the second fluid pressure threshold.

Preferably, according to another embodiment, the predetermined threshold is greater than the first fluid pressure threshold.

Preferably, according to another embodiment, the fluid-tight chamber comprises a release valve configured to release the fluid flowing within the fluid-tight chamber out of the fluid-tight chamber.

Preferably, according to another embodiment, the release valve is configured to release the fluid that flows from the fluid pressure power source and accumulates within the fluid-tight chamber out of the fluid-tight chamber to the ambient around the cassette or a receiving container included in the cassette when the fluid pressure reaches a predetermined threshold.

Preferably, according to another embodiment, the release valve is connected to an emergency button. The release valve is configured to release the fluid flowing within the fluid-tight chamber out of the fluid-tight chamber and to the ambient around the cassette or a receiving container included in the cassette when the emergency button is activated, thus, to cease the motivation of the medicament contained within the container.

Preferably, according to another embodiment, the emergency button can be pressed, or pulled, or slid, or twisted relative to the container carrier or the reusable body of the medicament delivery device for activating the emergency button.

Alternatively, according to another embodiment, the release valve is configured to slow down the medicament delivery rate.

Alternatively, according to another embodiment, the release valve is connected to a turned orifice.

Preferably, according to another embodiment, the fluid-tight chamber of the container carrier is at least partially made of rigid material.

Preferably, according to another embodiment, the fluid-tight chamber of the container carrier formed by a container frame configured to be attached to the medicament container; and an interior chamber of the container carrier. The container frame is configured to surround the medicament container.

Alternatively, or additionally, according to another embodiment, the fluid-tight chamber of the container carrier formed by a container frame configured to be attached to the medicament container; and a cap configured to be attached to the container frame. The container frame is configured to surround the medicament container.

Preferably, according to another embodiment, the cap comprises the tube set.

Preferably, according to another embodiment, when the cap is attached to the container frame, the delivery tube is fluidly connected to the medicament container surrounded by the container frame.

Preferably, according to another embodiment, when the cap is attached to the container frame, the piercing member is configured to pierce the fluid outlet of the medicament container.

Alternatively, according to another embodiment, when the cap is attached to the container frame, the piercing member is configured to pierce the fluid outlet of the medicament container once a piercing trigger is activated.

Preferably, according to another embodiment, the fluid-tight chamber comprises a pressure sensor.

Preferably, according to another embodiment, the fluid-tight chamber is coupled to a fluid-tight measure chamber. The fluid-tight measure chamber is configured to be connected to the fluid pressure power source and is configured to be in the same fluid pressure level as the fluid-tight chamber. The fluid-tight measure chamber comprises a piston. The piston is configured to be operably connected to a position sensor configured to sense the position of the piston within the fluid-tight measure chamber.

Preferably, according to another embodiment, the position sensor of the piston is configured to monitor the pressure level within the fluid-tight chamber by monitoring the position of the piston.

Preferably, according to another embodiment, the container carrier comprises a position sensor configured to detect the position of the medicament container.

Preferably, according to another embodiment, the position sensor of the medicament container is configured to monitor the pressure level within the fluid-tight chamber by monitoring the position of the medicament container.

Another aspect of the invention provides a medicament delivery device comprising the cassette.

Preferably, according to another embodiment, the medicament delivery device is portable.

Preferably, according to another embodiment, the medicament delivery device is body worn, underneath or over clothing.

Preferably, according to another embodiment, the medicament delivery device is an injection device, e.g., an infusion device or an on-body injector.

Preferably, according to another embodiment, the medicament delivery member is an injection needle or an insertion needle with a soft cannula.

Preferably, according to another embodiment, the medicament delivery device comprises a reusable body and a changeable medicament delivery member.

Preferably, according to another embodiment, the reusable body of the medicament delivery device comprises the fluid pressure power source connected to the inlet of the fluid-tight chamber of the container carrier. The fluid outlet is operably connected to the medicament delivery member.

Preferably, according to another embodiment, the medicament delivery device comprises a processor electrically connected to the fluid pressure power source, an electrical power source connected to the processor. The processor and the electrical power source are accommodated within the reusable body.

Preferably, according to another embodiment, the fluid-tight chamber is adjacent to the fluid pressure power source. The fluid pressure power source is fluidly connected to the inlet of the fluid-tight chamber directly.

Preferably, according to another embodiment, a transmission tube is arranged between the fluid pressure power source and the inlet of the fluid-tight chamber. The fluid pressure power source is fluidly connected to the inlet of the fluid-tight chamber via the transmission tube.

Preferably, according to another embodiment, the transmission tube is attached to the container frame.

Preferably, according to another embodiment, the medicament delivery device is configured to be attached to two or more cassettes.

Preferably, according to another embodiment, the two or more cassettes are stacked on one another.

Preferably, according to another embodiment, the medicament delivery device is configured to be attached to the cassette comprising multiple medicament containers.

Preferably, according to another embodiment, the medicament delivery device comprises a user interface attached to the reusable body.

Preferably, according to another embodiment, the user interface is electrically connected to the processor.

Preferably, according to another embodiment, the user interface is a button protruding from an outer surface of the reusable body.

Preferably, according to another embodiment, the user interface is a screen or touch panel arranged on an outer surface of the reusable body.

Preferably, according to another embodiment, the medicament delivery device comprises a display.

Preferably, according to another embodiment, the medicament delivery device comprises an orientation sensor such that the display, the scree, or the touch panel can always be presented in the right-reading graphic indicia to a user.

Preferably, according to another embodiment, the medicament delivery device comprises a wireless communication receiver connected to the processor. The wireless communication receiver can be based on the technology of radio frequency identification (RFID), near-field communication (NFC), Bluetooth, Bluetooth low energy (BLE), Ultra wide band (UWB), wireless fidelity (Wi-Fi), cellular communication, and infrared (IR), as non-limiting examples.

Preferably, according to another embodiment, the medicament delivery device comprises a wireless communication transmitter connected to the processor. The wireless communication transmitter can be based on the technology of RFID, NFC, Bluetooth, BLE, UWB, Wi-Fi, cellular communication, and IR, as non-limiting examples.

Preferably, according to another embodiment, the wireless communication receiver is configured to receive a wireless signal from a remote device or an information tag.

Preferably, according to another embodiment, the wireless communication transmitter is configured to transmit a wireless signal to a remote device or an information tag. Preferably, according to another embodiment, the pressure sensor and/or the position sensor of the piston of the fluid-tight measure chamber and/or the position sensor of the container carrier is electrically connected to the processor.

Preferably, according to another embodiment, the processor is configured to control the fluid pressure power source to output fluid into the fluid-tight chamber according to a signal from the pressure sensor and/or the position sensor of the piston of the fluid-tight measure chamber and/or the position sensor of the container carrier.

Therefore, the medicament delivery device provides a medicament delivery system with real-time control of the medicament delivery operation. The system can have two control modalities for medication dispense: pressure-controlled and flow rate-controlled. In a pressure-controlled embodiment, a set pressure may be held in the fluid-tight chamber, allowing medicament outflow to be throttled by pressure on the medicament delivery site, e.g., a patient's subcutaneous backpressure, and/or the medicament, environment, or system parameters, e.g., a temperature-dependent viscosity decreasing the hydraulic resistance of the medicament as the medicament warms up due to environmental conditions.

Alternatively, when the fluid pressure power source is a pneumatic power source, the flow rate may be controlled in a closed-loop fashion using the Ideal Gas Law and/or its simplifications, namely Boyle's law and Charles's Law. The system may be configured to calculate (i.e., infer) the remaining medicament. The system may be configured to calculate (i.e., infer) the remaining dose-volume by measuring the pressure in a fixed internal volume fluid-tight chamber, thus calculating the void space within the fluid-tight chamber based on the mass of fluid that has flowed into the fluid-tight chamber. This is accomplished through continuous or periodic monitoring of the dispensing of each medication by the processor and at least one of the pressure sensor(s), the position sensor(s) of the piston of the fluid-tight measure chamber, and the position sensor(s) of the container carrier. It should be noted that, in this example, the flow rate means the rate of the flow that leaves the medicament container. The flow rate is not necessarily equal to a delivery rate of the medicament contained within the medicament container, namely the rate that the user receives medicament from the medicament delivery device (the medicament delivery rate). For example, when other rate control arranged is used, e.g., a pinch valve attached to the flexible tube, the delivery rate of the medicament contained might be different with the flow rate. Alternatively, if there is no other rate control arranged is used, for example, the flexible bag is attached to a medicament delivery member, e.g., a needle, in this example, the flow rate is substantially equal to a delivery rate of the medicament contained within the medicament container.

Furthermore, in the example where the fluid pressure power source is a pneumatic power source, the volume of the medicament within the medicament container may be determined using the Ideal Gas Law and its simplifications, e.g., Boyle's law and Charles's Law. Application of the Ideal Gas Law is critical to volumetric system actions, (flow rate control, medication volume sensing), as volumes of abstract geometry cannot be directly probed by known cost-effective sensing methods. For a commercial presentation of this device, it is unlikely, however, that the system use pure ideal gases; use of ambient air is highly advantageous. So, while the Ideal Gas Law, PV=nRT, applies directly to ideal gases, it is not a perfect representation of ambient air. Introducing a compressibility factor, z, to the Ideal Gas Law allows for general application to ambient air; PV=znRT. At the foreseeable system temperatures and pressures (280-310 K, 1-10 bar), 0.9992<z<1.0004, and therefore air can be approximated as an ideal gas. Alternatively, to compensate for the inherent sensitivities to environmental variables and improve the pump performance in the presence of environmental uncertainty, the medicament delivery device may optionally comprise a “compensation block,” comprising an atmospheric pressure sensor and/or an ambient temperature sensor such that the calculation can be calibrated based on the detection from the compensation block.

To further simplify the equation, it is evident that the temperature term T, does not have great influence, since the foreseeable values are in a tight window of 280-310K. Even in the event that the volume of air starts at 280K and rises to 310K over the course of a volume-sensing action, the temperature term alone could not cause more than a +/−5% reduction in volumetric assessment accuracy. That is a very conservative case, as the control air temperature is likely to be dominated by the medicament container enclosure temperature and indirectly by ambient room temperature. Furthermore, as the container carrier can be vented to the ambient air (by the release valve, for example), the air within the container carrier would be approximately equivalent to room temperature, which would not introduce any temperature-based inaccuracies. Allowing +/−5% measurement sensitivity in this step is likely acceptable clinically, and notably, a much greater sensitivity than volume verification methods in current pharmacy practice, which contain no capability of empirical assessment. However, introduction of a system air temperature sensor, or a local container carrier air temperature sensor would remove uncertainty due to a temperature difference between room-temperature air and the cassette's internal air. Obviously, although injections and infusions are used herein, accuracy of the system may be determined on a case by case basis, given a physiologic route of administration and appropriate clinical parameters thereto.

In a preferred system control model, the reduced Ideal Gas Law becomes PV∝nR. Since nR represents the number of molecules in the system, or mass, m, the reduction is therefore PV∝m; V∝m/P. To determine void volume of the system at any given time, the system must keep track of the transferred mass to the control volume, e.g., via a known relationship between the drive parameters and the injection mass or via a comparative, proxy control region of known volume that air is dispensed from, such as a traditional accumulator model. In a volume assessment step, it is critical that the unfilled volume of the medicament cassette and the device's internal air volume is known. This is because the volume assessment device is assessing the system's air volume, and the filled volume of medicament is equal to the difference between an unfilled system's air volume and the filled system's air volume.

The ability to determine the volume of the medicament within the medicament container is particularly advantageous as the system is insensitive to initial fill, and can determined based only on the change in mass and without knowing the initial fill volume (i.e., no programming step is required). As such, the direct calculation of the volume of medicament in the medicament container allows the unique benefit of a blind, third-party verification of medicament contained within the medicament container before use. Thus, the initial condition of the medicament container can be decided.

Furthermore, in another example, the flow rate is not sensed directly. Rather, the system repeatedly calculates the void volume of the system. Provided the only way void volume can change is by liquid leaving the medicament container, the rate of change of the void volume equates to the liquid flow rate.

To avoid noise observed in the liquid flow calculation when adjacent void volume measurements are used, it is possible to filter the measurements to obtain a cleaner signal. One such approach involves a buffer and linear regression. At each controller evaluation, the initial condition of the container carrier (calculated by the Ideal Gas Law and its simplifications) is added to a buffer and a linear regression is run on that buffer. The slope of the regression line is the flow rate.

The system allows for continuous control of flow rate: either seeking a target flow rate by making continuous fluid delivery into the fluid-tight chamber while monitoring pressure, by removing the fluid flowed from the fluid pressure power source from the fluid-tight chamber while monitoring pressure, and/or by stopping flow abruptly (e.g., as in an emergency or during a systemic infusion reaction) by delivering the accumulated pressure or evacuating the fluid-tight chamber, reducing the pressure to ambient around the cassette or a receiving container included in the cassette and removing all forward fluid motivation. For example, the system can control the release valve to release the fluid to the ambient around the cassette or a receiving container included in the cassette based on a detection from one or more connected sensor(s). The system allows alteration of flow during medicament delivery operation, as may be needed during rate adjustment regimens common in oncology and provides for each cassette to have a desired flow rate that may be configured independently. Different medication cassettes or container carriers may be combined in a desired sequence, each cassette may have any desired fluid volume and may be delivered at a desired flow rate independently of viscosity, volume, or other medicament, patient, or system configuration (e.g., cannula gauge) parameters.

Furthermore, in another example, the system can be arranged with target maintenance mechanism. In this example, the target maintenance period simply adjusts the pressure target of the system proportionally to the measured flow rate error, as ideally there is a linear relationship between pressure and liquid flow rate. The flow rate error is interpreted as a ratio between target and measured liquid flow rate rather than a difference. The pressure target adjustment method may take many forms, but all inherently would be fed the flow error as an input for the response adjustment. Some controllers, such as Proportional Integral (PI) controller, Proportional-Integral-Derivative (PID) controller, or bang-bang controller can be used.

Furthermore, when the medicament delivery member is an injection needle or an insertion needle with a soft cannula, the system can also detect a needle removal when a pressure drop within the fluid-tight chamber is detected.

Preferably, according to another embodiment, an intentional breakpoint can be arranged close to the terminating needle end. For example, such that if the delivery tube were to be pulled in a fashion that would normally dislodge the needle from the patient skin, the tube would instead break at this joint separating the delivery tube from the needle end.

This break would have the effect of removing the pressure drop associated with the needle and subcutaneous tissue backpressure. Thus, for a medication delivered at a controlled flow rate, the upstream drive pressure would decrease.

With a drive system capable of continuously monitoring pressure and/or flow rate of medicament leaving the medicament container, these sudden flow changes would be detectable and would indicate detachment of the delivery tube from the terminating needle, presenting an error condition that would stop the injection.

Preferably, according to another embodiment, the fluid pressure power source is connected to the fluid-tight chambers of the two container carriers; and wherein the processor is configured to control the fluid pressure power source to selectively output fluid into at least one of the fluid-tight chambers.

Preferably, according to another embodiment, the processor is configured to control the fluid pressure power source to selectively output fluid into at least one of the fluid-tight chambers according to a signal from the pressure sensor and/or the position sensor of the piston of the fluid-tight measure chamber and/or the position sensor of the container carrier.

Preferably, according to another embodiment, the processor is configured to control the fluid pressure power source to output a certain amount of fluid. The certain amount is predetermined or is dependent on a signal from the pressure sensor and/or the position sensor of the piston of the fluid-tight measure chamber and/or the position sensor of the container carrier; thereby only the certain amount of medicament contained within the medicament container can be pressed out of the fluid outlet.

Preferably, according to another embodiment, the fluid pressure power source comprises a piezo pump configured to cause fluid to output from the fluid pressure power source. Furthermore, the piezo pump is preferably used as the pneumatic power source. Using a piezo pump can be advantageous as it can provide quiet or silent operation and can provide lower power operation when compared to motor-driven solutions.

Preferably, according to another embodiment, the fluid pressure power source comprises a motor-based fluid pump configured to cause fluid to output from the fluid pressure power source.

Preferably, according to another embodiment, the fluid pressure power source comprises a piston pump when lower cost or complexity is desired.

Preferably, according to another embodiment, when the fluid pressure power source is the pneumatic power source, the pneumatic power source is a well-controlled diaphragm pump when lower cost or complexity is desired.

Preferably, according to another embodiment, when the fluid pressure power source is the pneumatic power source, the pneumatic power source comprises an electronic engine, e.g., a MEMS engine, and a liquid substance. In this example, the engine is configured to trigger an electro-chemical reaction of the liquid substance to generate propellant gas.

Preferably, according to another embodiment, the fluid pressure power source a fluid pump having an inlet fluidly connected to the environment and an inlet filter connected to the inlet of the fluid pump comprises an inlet filter connected to the fluid pump such that contamination from the environment, e.g., dust, can be prevented from entering to the fluid pump.

Preferably, according to another embodiment, the fluid pressure power source comprises a pump-outlet check valve, followed by a downstream controllable release valve (venting to atmosphere), a flow sensor, a pressure sensor, an outlet filter connected to the fluid-tight chamber.

Preferably, according to another embodiment, the fluid pressure power source comprises a pressurized fluid canister configured to cause fluid to output from the fluid pressure power source.

Preferably, according to another embodiment, when the fluid pressure power source is the pneumatic power source, the pneumatic power source comprises a pressurized gas canister.

Preferably, according to another embodiment, the medicament delivery device is an infusion device.

Preferably, according to another embodiment, the fluid pressure source is a pneumatic power source. In this embodiment, the fluid outputted from the fluid pressure power source is gas, e.g., air or nitrogen.

Preferably, according to another embodiment, the fluid pressure source is a hydraulic power source. In this embodiment, the fluid outputted from the fluid pressure power source is liquid, e.g., water or oil.

Furthermore, another aspect of the invention provides a method for controlling a medicament delivery device. The medicament delivery device comprises the fluid pressure power source being a pneumatic power source as mentioned in any of the above-mentioned embodiments. The medicament delivery device comprises the fluid-tight chamber as mentioned in any of the above-mentioned embodiments, the fluid-tight chamber contains a medicament container. The medicament container is the flexible bag having the fluid outlet. The flexible bag contains medicament. The method comprising the following steps in the following order:
receiving at least one of a pressure level measurement of fluid pressure within the fluid-tight chamber, and a flow rate measurement of the medicament leaving rate from the fluid outlet the flexible bag;
fetching information from a database with the received measurement; and
providing a signal based on the fetched information to make one or more electronic components of the medicament delivery device perform an action or stop the currently performing action of one or more electronic components of the medicament delivery device.

The method is configured to monitor the condition within the fluid-tight chamber by monitoring the pressure level within the fluid-tight chamber and/or the medicament leaving rate from the fluid outlet the flexible bag and adjust the medicament delivery operation accordingly. As a result, the pneumatic power source and the medicament delivery device is protected, as, for example, the overshooting situation of the pneumatic power source and/or the entire medicament delivery system can be avoided. Furthermore, the user behavior, e.g., premature medicament delivery member removal, can be track.

The method can also be used to probe the actual filled medicament within the medicament container, calculating the actual delivery medicament and/or the residual medicament within the medicament by comparing the initial probed volume of the medicament and the after-used probed volume of the medicament. It should be noted that as the medicament filled volume calculation is carried out based on the pressure level measurement of fluid pressure within the fluid-tight chamber; thus, the measurement can be either obtained by measure the pressure level within the fluid-tight chamber directly or calculated with the medicament leaving rate from the fluid outlet the flexible bag (a measurement of the flow rate). Furthermore, for calculating the medicament filled volume, the pressure level measurement of fluid pressure within the fluid-tight chamber can be positive pressure level or negative pressure level. For example, the pneumatic fluid power source can be controlled to input a quantity of fluid, e.g., gas, air, into the fluid-tight chamber for measuring the medicament filled volume (the quantity of fluid will be released from the release valve after the medicament filled volume calculation and before medicament delivery operation); alternatively, the pneumatic fluid power source can be controlled to pull out fluid, e.g., gas, air, between the fluid-tight chamber and the flexible bag as the medicament filled volume calculation is carried out based on the information of magnitude of vacuum.

Preferably, according to another embodiment, the step of receiving at least one of a pressure level measurement of fluid pressure within the fluid-tight chamber, and a flow rate measurement of the medicament leaving rate from the fluid outlet the flexible bag comprises a step of.
receiving a pressure level measurement of fluid pressure within the fluid-tight chamber, and a flow rate measurement of the medicament leaving rate from the fluid outlet the flexible bag.

Preferably, according to another embodiment, the step of fetching information from a database with the received measurement comprises the following steps in the following order:
calculating, based on the received measurement with the Ideal Gas Law and its simplifications, a value;
comparing the calculated value and a predetermined value; and
generating a compared result.

Preferably, according to another embodiment, the step of generating a compared result, the step of fetching information from a database with the received measurement comprises further comprises a step of: providing the fetched information by matching the compared result with information from the database.

Preferably, according to another embodiment, after the step of generating a compared result, the step of fetching information from a database with the received measurement comprises further comprises a step of: providing the fetched information by providing the compared result.

Preferably, according to another embodiment, the fetched information is about at least one of an actual filled volume of the medicament within the medicament container, a residual volume of the medicament within the medicament container after use, air in a delivery tube, a delivery member is away from a delivery site, and delivery occlusion.

Preferably, according to another embodiment, the predetermined value is about at least one of a volume of the medicament container, a volume of the medicament contained within the medicament container, a target flow rate of the medicament leaving rate from the fluid outlet the flexible bag, a target pressure level of fluid pressure within the fluid-tight chamber, a previously received flow rate of the medicament leaving rate from the fluid outlet the flexible bag, a previously received pressure level of fluid pressure within the fluid-tight chamber, previously calculated volume of the medicament contained within the medicament container.

Preferably, according to another embodiment, the predetermined value is received from an information tag on the medicament container.

Preferably, according to another embodiment, the action of the one or more electronic components of the medicament delivery device is configured to perform or stop by the provided signal is at least one of providing an indication to a user of the medicament delivery device, medicament delivery operation, sending data to a remote server, adjusting the pressure level of fluid pressure within the fluid-tight chamber, and adjusting the medicament leaving rate from the fluid outlet the flexible bag.

Preferably, according to another embodiment, a processor of the medicament delivery device as mentioned in any one of the embodiments above is configured to perform the method according to any one of the above-mentioned embodiments.

Preferably, according to another embodiment, the fluid-tight chamber of the cassette is operably connected to a pressure sensor configured to measure a pressure level measurement of fluid pressure within the fluid-tight chamber and/or a flow rate sensor configured to measure the medicament leaving rate from the fluid outlet the flexible bag.

Preferably, according to another embodiment, the cassette is operably connected to the pressure sensor and the flow rate sensor.

Preferably, according to another embodiment, the cassette comprises the pressure sensor and the flow rate sensor.

Preferably, according to another embodiment, the reusable body of the medicament delivery device comprises the processor.

Preferably, according to another embodiment, the processor electrically connected to the fluid pressure power source.

Preferably, according to another embodiment, the processor electrically connected to the pressure sensor and the flow rate sensor when the cassette is attached to the reusable body of the medicament delivery device.

Preferably, according to another embodiment, the reusable body of the medicament delivery device comprises a communication unit configured to read an information tag on the medicament cassette when the cassette is attached to the reusable body of the medicament delivery device.

Preferably, according to another embodiment, the communication unit is electrically connected to the processor.

Preferably, according to another embodiment, the communication unit is an RFID/NFC reader and/or RFID/NFC writer.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, small molecules, hormones, cytokines, blood products, antibodies, antibody-drug conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Exemplary drugs that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary drugs that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary drugs that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Exemplary drugs that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a cassette according to the invention, the cassette being connected to a fluid pressure power source.

FIGS. 2A-2B schematically shows a fluid-tight chamber of the cassette of FIG. 1. Arrows on FIG. 2A are used to show moving path of fluid from the fluid pressure power source. Arrows on FIG. 2B are used to show fluid path of the medicament from the medicament container.

FIGS. 3A-3B schematically shows the fluid-tight chamber of the cassette of FIG. 1 in another embodiment. Arrows on FIG. 3A are used to show moving path of fluid from the fluid pressure power source. Arrows on FIG. 3B are used to show moving path of fluid that is pulling out of the fluid-tight chamber by a vacuum device.

FIG. 4 schematically shows the cassette of FIG. 1 in another embodiment.

FIG. 5 schematically shows the cassette of FIG. 1 with a container carrier comprising multiple fluid-tight chambers.

FIG. 6 schematically shows the cassette of FIG. 1 with the container carrier comprising multiple fluid-tight chambers in another example.

FIG. 7 schematically shows the cassette of FIG. 1 connected to the fluid pressure power source via a transmission tube.

FIG. 8 schematically shows the fluid-tight chamber with a fluid-tight measure chamber.

FIG. 9 shows a perspective view of a medicament delivery device comprising the cassette of the invention.

FIG. 10 shows a perspective view of another medicament delivery device comprising the cassette of the invention.

FIG. 11 schematically shows the medicament delivery device of FIG. 9 in another embodiment.

FIG. 12 shows a perspective view of the fluid-tight chamber in one example.

FIG. 13 schematically shows the medicament delivery device with multiple fluid-tight chambers. Each of the fluid-tight chambers are connected to a fluid pressure power source directly.

FIG. 14 schematically shows the medicament delivery device with multiple fluid-tight chambers. One of the fluid-tight chambers are connected to a fluid pressure power source directly and the rest of the fluid-tight chambers are connected to a fluid pressure power source indirectly, via at least one of another fluid-tight chambers.

FIG. 15 shows a perspective view of a medicament delivery device in which the concepts described herein could be implemented. The medicament delivery device comprises a main body and a hinge lid.

FIG. 16 shows a perspective view of a medicament delivery device in which the concepts described herein could be implemented. The medicament delivery device comprises a reusable body with a side cavity that the cassette can be loaded into the reusable body from the side cavity.

FIG. 17 shows a perspective view of the medicament delivery device of FIG. 15. The medicament delivery device comprises a user wearable feature. The usable wearable feature is a strap.

FIG. 18 shows a perspective view of the medicament delivery device of FIG. 16. The medicament delivery device comprises a user wearable feature. The usable wearable feature is a strap.

FIG. 19 shows a perspective view of the medicament delivery device of FIG. 15. The medicament delivery device comprises a user wearable feature. The usable wearable feature is a belt clip.

FIGS. 20-23 show perspective views of a medicament delivery device in which the concepts described herein could be implemented. The medicament delivery device comprises a reusable body connected to multiple fluid-tight chamber and a user wearable feature attached to the reusable body. The usable wearable feature is a neck strap.

FIGS. 24-25C show perspective views of a medicament delivery device in which the concepts described herein could be implemented. The medicament delivery device comprises a reusable body connected to multiple fluid-tight chamber. The reusable body comprises a main body and a lid.

FIG. 26 shows a perspective view of the medicament delivery device of FIG. 16.

FIGS. 27-28 schematically show a fluid pressure power source of the invention.

FIGS. 29-30 schematically show the fluid pressure power source, and a medicament delivery device of the invention comprises a multi-ports valve.

FIGS. 31-34C show perspective views of cassettes of the invention in different examples.

FIG. 35 shows a control flow chart of the fluid pressure power source of the invention.

FIG. 36 shows a chart of the relation between the pressure and the flow rate.

FIGS. 37A-37B how perspective views of the medicament delivery device of the invention in another example.

### DETAILED DESCRIPTION

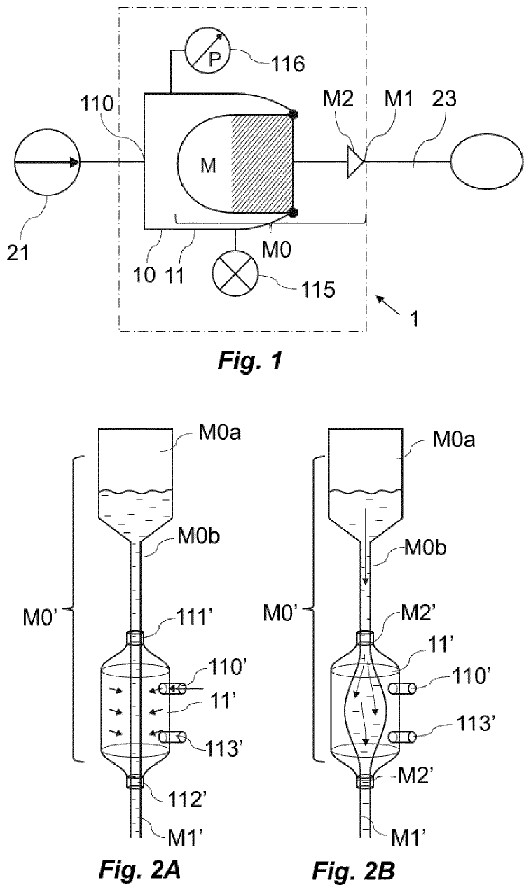

FIGS. 1-37B illustrate a number of example cassettes 1; 1″; 1′″ of a medicament delivery device 2; 2; 2″. The medicament delivery device 2; 2; 2″ comprises a reusable body 20; 20′; 20″; 20′″ comprising a fluid pressure power source 21. The fluid pressure power source can be a pneumatic power source or a hydraulic power source. The pneumatic power source is configured to output gas to the cassette for expelling contained medicament by increasing the fluid pressure of the gas; and the hydraulic power source is configured to output liquid to the cassette for expelling contained medicament by increasing the fluid pressure of the liquid. In a preferred example, the medicament delivery device 2 is a portable device. The cassette 1; 1″; 1′″ comprises a container carrier 10 and a medicament container M; Ma, Mb, Mc, Md; Ma′, Mb′, Mc′, Md′.

The medicament container M; Ma, Mb, Mc, Md; Ma′, Mb′, Mc′, Md′ is configured to be at least partially accommodated within the container carrier 10; 10′; 10″; 10′″; 10a″, 10b″, 10c″, 10d″. The medicament container M; Ma, Mb, Mc, Md; Ma′, Mb′, Mc′, Md′ comprises a body M0; M0′; M0″; M0a, M0b; M0a, M0a′, M0a″, M0b, M0b′; M0b″ and a fluid outlet M1; M1′; Ma1, Mb1, Mc1. The body M0; M0′; M0″; M0a, M0b; M0a, M0a′, M0a″, M0b, M0b′; M0b″ is configured to accommodate medicament. The medicament contained within the body MO; M0′; M0″; M0a, M0b; M0a, M0a′, M0a″, M0b, M0b′; M0b″ is configured to move out of the body M0; M0′; M0″; M0a, M0b; M0a, M0a′, M0a″, M0b, M0b′; M0b″ through the fluid outlet M1; M1′; M1″; Ma1, Mb1, Mc1.

Each component of the cassette, including optional components, will first be explained in detail. Examples of cassettes comprising different combinations of the described components will be explained in detail later.

The body M0; M0′; M0″; M0a, M0b; M0a, M0a′, M0a″, M0b, M0b′; M0b″ comprises a flexible portion. In a preferred example, the body M0; M0′; M0″; M0a, M0b; M0a, M0a′, M0a″, M0b, M0b′; M0b″ comprises a flexible bag and/or a flexible tube. It should be noted that, in some examples below, the term ‘delivery tube’ and the term ‘flexible tube’ are used interchangeably, when the delivery tube is mentioned to be flexible. In other words, when the delivery tube is flexible, the delivery tube can be the flexible tube of the body of the medicament container. In one example, the body MO comprises the flexible bag M0a configured to accommodate the medicament, as shown in FIGS. 2A-2B. In this example, the flexible portion of the body MO is defined by the flexible bag. In this example, the fluid outlet M1 can be connected directly to a medicament delivery member 23 of the medicament delivery device, e.g., a steel injection needle, or the fluid outlet M1 is configured to be operably connected to the medicament delivery member 23. For example, the fluid outlet M1 is operably connected to one end of a delivery tube 31, and the other end of the delivery tube 31 is connected to the medicament delivery member 23, as shown in FIGS. 32-34C, or the body comprises a rigid delivery tube connected to the body MO. The rigid delivery tube defines the fluid outlet M1.

Alternatively, the body M0′ comprises the delivery tube M0b, in this example, the delivery tube M0b is flexible. In this example, the flexible portion of the body M0′ is defined by a portion of the delivery tube M0b. In this example, the body M0′ can be partially made of a rigid material, e.g., a glass ampule and can be assembled/integral with the delivery tube M1′. In this example, the delivery tube M1′ defines the fluid outlet M1, meaning that the flexible delivery tube M1′ can be connected to the medicament delivery member 23.

In a preferred example, the body of the medicament container comprises the flexible bag received within the fluid-tight chamber.

In a preferred example, the body of the medicament container comprises the flexible bag and the flexible tube; or the body of the medicament container comprises the flexible bag configured to be operably connected to a flexible tube 31 (not a part of the body). In this example, regardless of whether the flexible portion of the body that is configured to be pressed by the fluid from the fluid pressure power source is a part of the flexible bag or a part of the flexible tube (when the body comprises the flexible tube), the delivery rate of the medicament contained within the medicament container can be controlled by manipulating the flexible tube. For example, a pinch valve can be attached to the flexible tube, so that the delivery rate of the medicament contained within the medicament container (the medicament delivery rate) can be adjusted via the pinch valve. Preferably, in this example, a flowmeter can be connected to the flexible tube for measuring the delivery rate of the medicament contained within the medicament container. The flowmeter can be a pinwheel sensor, an ultrasound sensor and/or a calorimetric sensor. It should be noted that the medicament delivery rate might not be equal to the flow rate of the medicament leaves the medicament container (the flow rate) as the rate control arrangement may be positioned outside of the medicament container, e.g., a pinch valve is attached to the flexible tube that is not a part of the body of the medicament container.

In one example where the body comprises the delivery tube, or the body of the medicament container comprises the flexible bag configured to be operably connected to a flexible tube 31 (not a part of the body), the delivery tube optionally comprises a tube valve M2. Additionally, in a preferred example, the tube valve is a one-way valve M2 so that medicament flowing in the delivery tube cannot flow back towards the body of the medicament container M, instead, the medicament flowing in the delivery tube can only flow towards the fluid outlet M1; M1′; Ma1, Mb1, Mc1. Additionally, in a preferred example, the tube valve M2 of the delivery tube is an umbrella valve, a spring-based valve or a ball valve. It should be noted that the tube valve is a useful safety arrangement; however, it is not a necessary component for the cassette of the disclosure. For example, when the medicament container is configured to be upright relative to the ground, namely that the fluid outlet is aiming towards the ground, during use of the medicament delivery device comprising the cassette of the disclosure, the gravity of the medicament within the body may prevent the medicament from flowing back towards the body.

Alternatively, when the medicament container is configured to be subjected to pressure that is higher enough to expel the medicament out from the fluid outlet, the pressure may also prevent the medicament from flowing back towards the body.

In one example, the container carrier 10; 10′; 10″; 10′″; 10a″, 10b″, 10c″, 10d″ comprises a fluid-tight chamber 11; 11′; 11″; 11a′″, 11b′″, 11c′″; 11a″″, 11b″″, 11c″″, 11d″″; 11′″″, 11″″″, 11a′″″, 11b′″″, 11c′″″, 11d′″″; 11a′″″″, 11b′″″″, 11c′″″″, 11d″″″″. In this example, the medicament delivery device can be easily to be used by the end user, as most of the connection between components are preassembled, the end user will only need to attach the cassette to the power source 21 and connect the delivery member 23 to the cassette. In one example, the fluid-tight chamber 11a′″″″″, 10e′″″; 11a″″″″″, 11b″″″″″ of the container carrier formed by a container frame 11a′″″″″; 11a″″″″″ configured to be attached to the medicament container M, as shown in FIG. 32 and FIGS. 34A-34C. In one example, as shown in FIG. 32, the fluid-tight chamber 11a′″″″″, 10e′″″of the container carrier is formed by the container frame 11a′″″″″ and an interior chamber 10e′″″ of the container carrier 10′″″. Alternatively, or additionally, the fluid-tight chamber 11a″″″″″, 11b″″″″″ of the container carrier is formed by the container frame 11a′″″″″ and a cap 11b″″″″″ configured to be attached to the container frame 11a″″″″″.

The container frame 11a′″″″″; 11a″″″″″ is configured to surround the medicament container M. In one example, the container frame 11a″″″″ is configured to surround the outer contour of the medicament container M, as shown in FIG. 32. This example is suitable when the medicament container is a flexible bag. As the container frame supports the outer contour of the medicament container, the risk of kinking during filling the medicament can be reduced. In this example, the container frame 11a″″″″ can be form in a shape that is matched to the outer contour of the flexible bag. For example, if the flexible bag is generally rectangular, the container frame can be a generally rectangular ring configured to closely surrounds the outer contour of the flexible bag.

Alternatively, or additionally, the container frame 11a″″″″″ is configured to receive a part of the medicament container M, as shown in FIGS. 34A-34C. In this example, the container frame comprises a cavity to receive the flexible bag at least partially. In this example, comparing to the previous example, more outer surface area of the flexible bag will be surrounded by the container frame.

Preferably, the cap comprises a tube set 3 (will be explained in detail later). In a preferred example, the medicament container M comprises a fixture M4 configured to engage with one or more counter fixtures 30a, 30b of the cap. For example, the fixture M4 is a flange, and the counter fixture 30a, 30b is a protrusion. In one example as shown in FIGS. 34A-34C, the cap comprises a first set of counter fixture 30a positioned closer to a piercing member 32 (will be explained in detail later); and a second set of counter fixture 30b. In this example, the medicament container M is configured to be attached to the second set of counter fixture 30b, as shown in FIG. 34B, and pushed to engage with the first set of the counter fixture 30a before use, as shown in FIG. 34C. As a result, the fluid connection between the delivery tube 31 and the medicament container M is established. For example, the medicament container M can be pre-attached to the second set of counter fixture 30b, as shown in FIG. 34B, such that the user can simply push the medicament container to be engaged with the first set of counter fixture 30a before use.

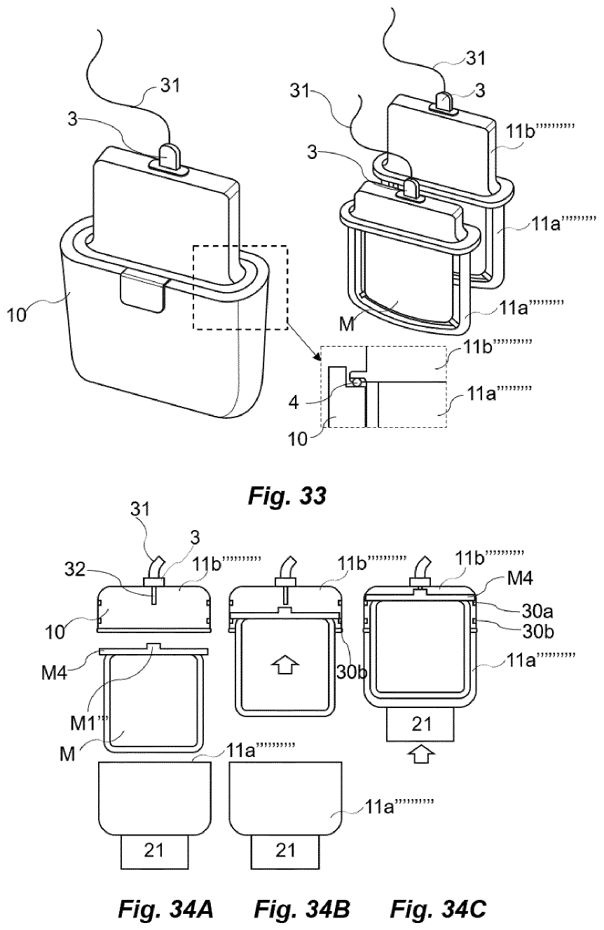

In one example, the cap 11b″″″″″ and the container frame 11a″″″″″ form the fluid-tight chamber, as shown in FIGS. 34A-34C. Alternatively, the cap 11b″″″″″, the container frame 11a′″″″″, and the interior chamber 10e′″″of the container carrier 10′″″ form the fluid-tight chamber as shown in FIG. 33. Alternatively, the container frame 11a′″″″″, and the interior chamber 10e′″″ of the container carrier 10′″″ form the fluid-tight chamber as shown in FIG. 32.

The pre-attachment can be done by a caregiver or in production line. The cap is configured to seal the container frame to form the fluid-tight chamber. In a preferred example, the cap is attached to the container frame via a thread engagement, a snap-fit engagement, or being clamped. In a preferred example, a rubber ring 4 is positioned between the cap 11b″″″″″ and the container frame 11a′″″″″ to enhance the fluid-tight seal, dependent on what level of fluid-tight seal is needed for the design. The cap can be dome shape with the delivery tube extending through the center of the dome shape cap, or the cap can be any suitable shape dependent on the design of container frame and/or the container carrier.

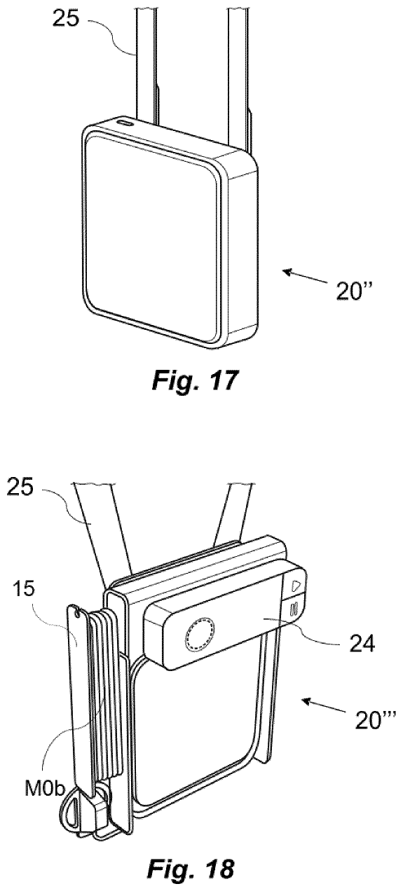

In another example, the container carrier 10; 10′; 10″; 10′″; 10a″, 10b″, 10c″, 10d′ does not completely comprise the fluid-tight chamber 11; 11′; 11″; 11a′″, 11b′″, 11c′″; 11a″″, 11b″″, 11c″″, 11d″″; 11′″″, 11″″″, 11a′″″″, 11b′″″″, 11c′″″″, 11d′″″″; 11a″″″″, 11b″″″″, 11c″″″″, 11d″″″″. Instead, the fluid-tight chamber 11; 11′; 11″; 11a′″, 11b′″, 11c′″; 11a″″, 11b″″, 11c″″, 11d″″; 11′″″, 11″″″, 11a′″″″, 11b′″″″, 11c′″″″, 11d′″″″; 11a″″″″, 11b″″″″, 11c″″″″, 11d″″″″ is formed by a combination of the container carrier 10; 10′; 10″; 10′″; 10a″, 10b″, 10c″, 10d′ and a part of the medicament delivery device 2 that comprises the cassette, meaning that the container carrier of the cassette partially comprises the fluid-tight chamber 11; 11′; 11″; 11a′″, 11b′″, 11c′″; 11a″″, 11b″″, 11c″″, 11d″″; 11′″″, 11″″″, 11a′″″″, 11b′″″″, 11c′″″″, 11d′″″″; 11a″″″″, 11b″″″″, 11c″″″″, 11d″″″″ and the medicament delivery device also partially comprises the fluid-tight chamber 11; 11′; 11″; 11a′″, 11b′″, 11c′″; 11a″″, 11b″″, 11c″″, 11d″″; 11′″″, 11″″″, 11a′″″″, 11b′″″″, 11c′″″″, 11d′″″″; 11a″″″″, 11b″″″″, 11c″″″″, 11d″″″″. In other words, the fluid-tight chamber 11; 11′; 11″; 11a′″, 11b′″, 11c′″; 11a″″, 11b″″, 11c″″, 11d″″; 11′″″, 11″″″, 11a′″″″, 11b′″″″, 11c′″″″, 11d′″″″; 11a″″″″, 11b″″″″, 11c″″″″, 11d″″″″ is formed only when the cassette is attached to a part of the medicament delivery device, as shown in FIG. 16, for example. In this example, the container carrier 10; 10′; 10″; 10′″; 10a″, 10b″, 10c″, 10d′ can be a chamber or a frame (will explained in detail later). In this example, the medicament delivery device can be compact, and the material cost can be reduced. Furthermore, as the cassette comprises the container carrier 10; 10′; 10″; 10′″; 10a″, 10b″, 10c″, 10d′, multiple connecting ports, such as connecting ports to multiple delivery members and/or multiple connecting ports to the power source 21 can be provided by the container carrier. Thus, the end user can easily connect to the power source and/or the delivery member to the medicament container.

The fluid-tight chamber 11; 11′; 11″; 11a′″, 11b′″, 11c′″; 11a″″, 11b″″, 11c″″, 11d″″; 11′″″, 11″″″, 11a′″″″, 11b′″″″, 11c′″″″, 11d′″″″; 11a″″″″, 11b″″″″, 11c″″″″, 11d″″″″ comprises an inlet 110; 110′; 110″; 110′″; 110″″; 110a. The inlet 110; 110′; 110″; 110′″; 110″″; 110a is configured to fluidly connect to an outlet 210 of the fluid pressure power source 21 of the reusable body of the medicament delivery device 2. It should be noted that when the fluid pressure power source is the pneumatic power source, the fluid-tight chamber is gas-tight; and when the fluid pressure power source is the hydraulic power source, the fluid-tight chamber is gas-tight or liquid-tight.

The fluid-tight chamber 11; 11′; 11″; 11a′″, 11b′″, 11c′″; 11a″″, 11b″″, 11c″″, 11d″″; 11′″″, 11″″″, 11a′″″″, 11b′″″″, 11c′″″″; 11d′″″″, 11a″″″″, 11b″″″″, 11c″″″″, 11d″″″″ is coupled to the flexible portion of the body M0; M0′; M0″; M0a, M0b; M0a, M0a′, M0a″, M0b, M0b′; M0b″ of the medicament container M: M′ so that when the inlet 110; 110′ receives output fluid from the fluid pressure power source 21, the output fluid flows into the fluid-tight chamber 11; 11′; 11″; 11′″ and the medicament contained within the medicament container M; Ma, Mb, Mc, Md; Ma′, Mb′, Mc′, Md′ is pressed out under the pressure of the output fluid M1; M1′.

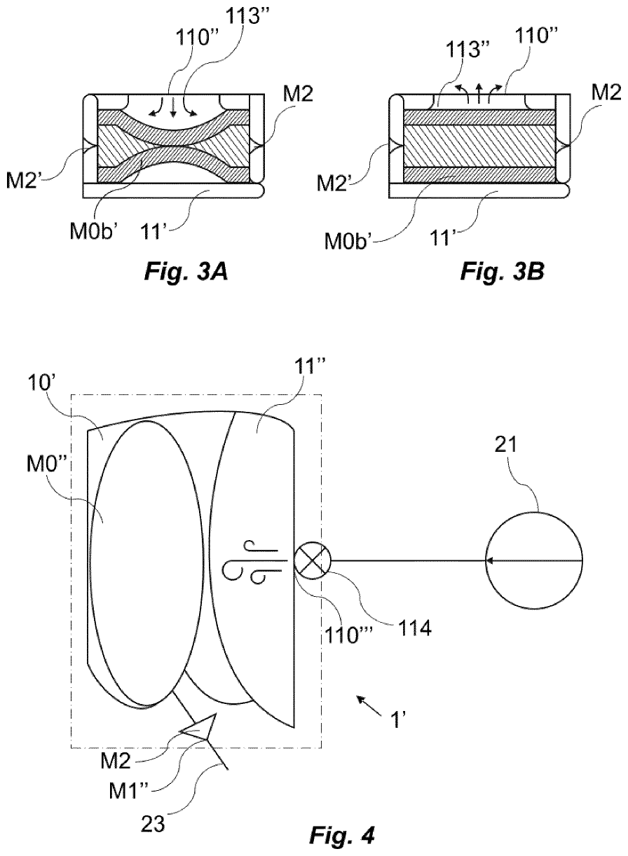

In one example, the flexible portion of the body M0; M0′; M0a, M0b; M0a, M0a′, M0a″, M0b, M0b′; M0b″ is at least partially accommodated within the fluid-tight chamber 11; 11′; 11a′″, 11b′″, 11c′″; 11a″″, 11b″″, 11c″″, 11d″″; 11′″″, 11″″″, 11a′″″″, 11b′″″″, 11c′″″″, 11d′″″″; 11a″″″″, 11b″″″″, 11c″″″″, 11d″″″″. In one example, the entire body MO can be arranged within the fluid-tight chamber 11′″″, as shown in FIG. 8 and FIG. 32. In one example, the fluid outlet M1 extends out of the fluid-tight chamber 11′″″. Alternatively, the fluid outlet M1′″ is fluidly connected to the delivery tube 31″, as shown in FIG. 34C. In these two examples, the body MO comprises the flexible bag. In one example, when the body comprises the delivery tube M0b′, and the delivery tube M0b′ is flexible, as shown in FIG. 2A, the fluid-tight chamber 11′ comprises a tube inlet 111′ and a tube outlet 112′. In this example, the delivery tube is configured to be positioned between the tube inlet 111′ and the tube outlet 112′. In a preferred example, the delivery tube M0b comprises two tube valves M2, M2′. In a preferred example, the fluid-tight chamber 11′ is configured to surround a portion of the delivery tube between the two tube valves M2, M2′, as shown in FIGS. 3A-B.

Alternatively, as shown in FIG. 4, the fluid-tight chamber is inflatable 11″. The fluid-tight chamber 11″ is adjacent to the flexible portion of the body MO, so that the output fluid from the fluid pressure power source is configured to flow into the fluid-tight chamber 11″ and inflate the fluid-tight chamber 11″ to press on the medicament container M. In one example as shown in FIG. 4, the body M0″ comprises the flexible bag, the fluid-tight chamber 11″ is configured to be inflated and thereby press on the flexible bag when the output fluid from the fluid pressure power source flows into the fluid-tight chamber 11″, therefore, the medicament contained within the flexible bag can be expelled. In this example, the flexible bag can be connected to a medicament delivery member directly or the flexible bag can comprise a flexible tube extending out of the fluid-tight chamber 11″ and/or the container carrier. Alternatively, when the body comprises the flexible tube, the fluid-tight chamber 11″ is configured to be inflated and thereby press on the flexible tube when the output fluid from the fluid pressure power source flows into the fluid-tight chamber 11″. In a preferred example, the fluid-tight chamber is also a tube. In this example, the fluid-tight chamber is attached to one or more flexible tube of the body of the medicament container. In this example, a multiple lumens medicament delivery tube is formed by at least one fluid-tight chamber tube and at least one flexible tube of the body of the medicament container.

In this example, one lumen of the multiple lumens medicament delivery tube is connected to the fluid pressure power source and the other one lumen of the multiple lumens is connected to the medicament container.

In this example, the container carrier comprises a rigid container chamber where the flexible portion of the medicament container and the fluid-tight chamber is at least partially located within the rigid container chamber of the container carrier.

The inlet 110; 110′; 110″; 110′″; 110″″; 110a of the fluid-tight chamber is configured to be connected to the fluid pressure power source 21. For example, the outlet 210 of the fluid pressure power source 21 can be connected to the inlet 110; 110′ of the fluid-tight chamber directly, or the outlet of the fluid pressure power source 21 can be connected to the inlet of the fluid-tight chamber through a transmission tube 22. In one example, the inlet 110′″ of the fluid-tight chamber comprises a valve 114; 114′. Alternatively, when the outlet of the fluid pressure power source 21 comprises a valve, the inlet of the fluid-tight chamber does not need to comprise the valve. The valve can be a multi-direction valve 114′ (will be explained in detail later). Alternatively, the valve 114 can be a one-way valve so that the fluid output from the fluid pressure power source 21 can only flow towards the fluid-tight chamber. In one example, the fluid-tight chamber is at least partially made of rigid material. In one example where the fluid-tight chamber is at least partially made of rigid material, the fluid-tight chamber is made of plastic material with structural enhancement to be a wall of the fluid-tight chamber, such as a honeycomb matrix. In other words, the wall of the fluid-tight chamber is not flat, instead, a honeycomb matrix or multiple ribs protrusion is provided. When the fluid-tight chamber is made of rigid plastic, the hard plastic wall can be fragile; for example, the hard plastic is easy to crack when the cassette or the medicament delivery device comprising the cassette falls and hits on a hard surface, e.g., the flat, hard ground, for example. The structural enhancement, such as the honeycomb matrix, makes the plastic wall be more flexible; thus, the plastic wall can be more robust. Alternatively, the fluid-tight chamber is a secondary flexible bag 11″″ that fully encloses the medicament container, as shown in FIG. 12. This could be accomplished via a multiple-layer bag assembly with one port as the fluid outlet and the other port as the inlet of the fluid-tight chamber. Alternatively, or additionally, the fluid-tight chamber can be cylindrical; due to the self-reinforcing nature of cylinders under internal pressure, a cylindrical fluid-tight chamber can achieve a high degree of structural stability under pressure without excessive use of plastic.

In another example, as shown in FIG. 1, the fluid-tight chamber 11 comprises a release valve 115 configured to release the fluid that flows into the fluid-tight chamber 11 out of the fluid-tight chamber 11. The release valve is an optional, safety design that is configured to immediately stop the medicament delivery operation. For example, the release valve 115 can be connected to a user-accessible emergency button and can be either a part of the cassette or a part of the reusable body 20; 20′; 20″; 20′″ of the medicament delivery device. Therefore, when the user needs to stop the medicament delivery operation, e.g., due to an adverse drug reaction, the user can activate, e.g., push/pull, the emergency button to open the release valve 115. Therefore, the fluid flows within the fluid-tight chamber can be released immediately and thereby the medicament delivery operation can be stopped. Additionally, the release valve 115 can also be designed to release the fluid that flows into the fluid-tight chamber out of the fluid-tight chamber when the fluid pressure reaches a predetermined threshold. Therefore, the pressure within the fluid-tight chamber can be controlled under a safety pressure value, e.g., a pressure value that will not damage the medicament delivery device and/or the medicament container.

Furthermore, the release valve can be configured to slow down the medicament delivery rate. In this example, a partial cassette depressurization mechanism can be provided. This effect is intentional, controlled, and has the effect of reducing or re-increasing (by closing the valve) the flow rate of the medicament leaving the medicament container. In a preferred example, the release valve is connected to a turned orifice.

Furthermore, in one example where the body M0a, M0b comprises the flexible tube, the fluid-tight chamber 11′ comprises an outlet 113′ configured to be connected to a vacuum device, so that when the pressure within the fluid-tight chamber is reduced, medicament contained within the medicament container is sucked into the flexible tube, as shown in FIGS. 2B and 3B. In this example, the delivered dosage can be more accurate. In this example, a certain amount of the medicament is configured to be sucked into the flexible tube when the pressure of the fluid-tight chamber 11′ is reduced, as shown in FIGS. 2B and 3B; this certain amount of the medicament is then configured to be expelled out of the fluid outlet when the fluid flows into the fluid-tight chamber 11′, as shown in FIGS. 2A and 3A. Preferably, in this example, the flexible tube comprises two tube valves M2, M3 positioned between the tube inlet of the fluid-tight chamber 11′ and the tube outlet of the fluid-tight chamber 11′. Therefore, any potential leakage of the medicament within the fluid-tight chamber 11′ that might impact the dose accuracy can be avoided. In one example, as shown in FIGS. 2A-3B, the fluid-tight chamber 11′ comprises an opening as the inlet 110 and another opening as the outlet 113′. Alternatively, the inlet of the fluid-tight chamber is the outlet of the fluid-tight chamber. In this example, the medicament delivery device comprises a ‘Y’ shape tube with a main tube portion connected to the fluid-tight chamber and two split tube portions. One of the split tube portions connects to the vacuum device and the other one connects to the pneumatic fluid source. Two valves can be positioned within the two split tube portions respectively. It should be noted that, the vacuum device and the fluid pressure power source can be two separable devices, e.g., two independent pump, or one pump and one ventilator. Alternatively, the vacuum device can be a part of the fluid pressure power source; for example, the fluid pressure power source can be a reversible pump.

In another example, the fluid-tight chamber 11 comprises a pressure sensor 116, as shown in FIG. 1. Alternatively, the fluid-tight chamber 11′″″ chamber is coupled to a fluid-tight measure chamber 13, as shown in FIG. 8. In this example, the container carrier 10′″ comprises the fluid-tight measure chamber 13 and the fluid-tight chamber 11. The fluid-tight measure chamber 13 is configured to be connected to the fluid pressure power source 21 and is configured to be in the same fluid pressure level as the fluid-tight chamber 11′″″. The fluid-tight measure chamber comprises a piston 130. The piston 130 is configured to be operably connected to a position sensor configured to sense the position of the piston 130 within the fluid-tight measure chamber 13. In this example, the position sensor can be either a part of the cassette or a part of the medicament delivery device. In this example, the pressure within the fluid-tight chamber can be measured based on the sensed position of the piston 130 of the fluid-tight measure chamber 13. Alternatively, the container carrier comprises a position sensor configured to detect the position of the medicament container. In this example, the pressure within the fluid-tight chamber can be measured based on the sensed position of the medicament container. For example, the position sensor is configured to continuously capture images of the medicament container. Therefore, the pressure level within the fluid-tight chamber can be calculated based on the image recognition technology with the images of the medicament container, e.g., indicating a different level of deformation. Additionally, the medicament cassette optionally comprises a flow sensor.

The cassette 1; 1′; 1″ is configured to be attached to the reusable body 20; 20′; 20″; 20′″ of the medicament delivery device 2. Preferably, the cassette 1; 1′; 1″ is configured to be releasably attached to the reusable body of the medicament delivery device 2. In this example, the user can exchange with a new cassette and return the used cassette after use. This example would be suitable for a user who needs to get regular medicament delivery. Alternatively, the cassette can be undetachably (for the user of the medicament delivery device) attached to the reusable body of the medicament delivery device. In this example, the user can return the entire medicament delivery device after use. This example would be suitable for a user who needs to receive a highly regulated medication, e.g., the medicament that is toxic or addictive. In one example, the cassette can be locked to the reusable body via a magnetic lock that can only be released by a certain tool, e.g., a predetermined magnet array.

When the cassette 1; 1′; 1″ is attached to the medicament delivery device 2, the fluid outlet M1; M1′; Ma1, Mb1, Mc1 is configured to be connected to the medicament delivery member 23 of the medicament delivery device 2.

In one example, the cassette comprises a cassette housing. In this example, the container carrier is arranged within the cassette housing. Alternatively, the container carrier is configured to be operably attached to the reusable body of the medicament delivery device. In this example, the cassette does not need the cassette housing. These two examples are suitable for the container carrier comprises a flexible part. For example, when the fluid-tight chamber is made of a secondary flexible bag.

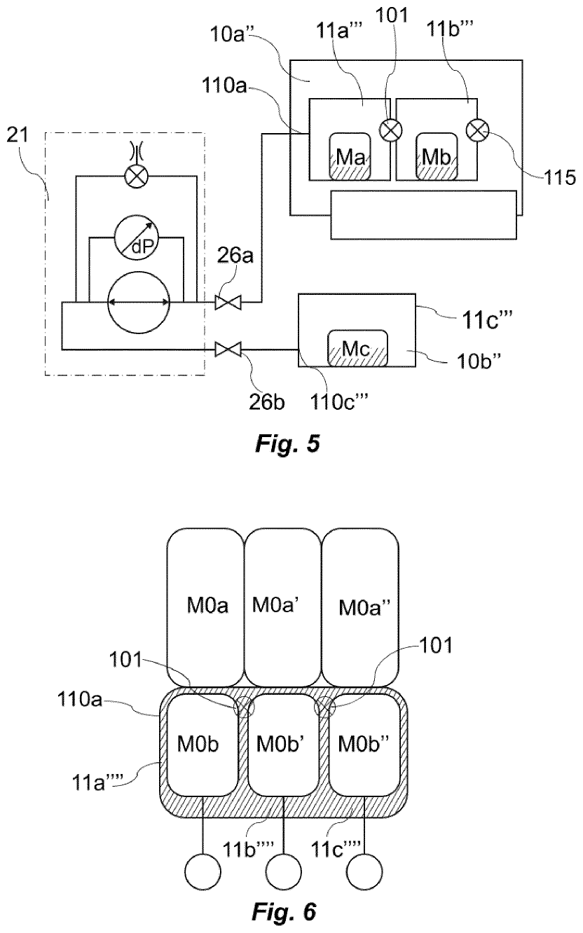

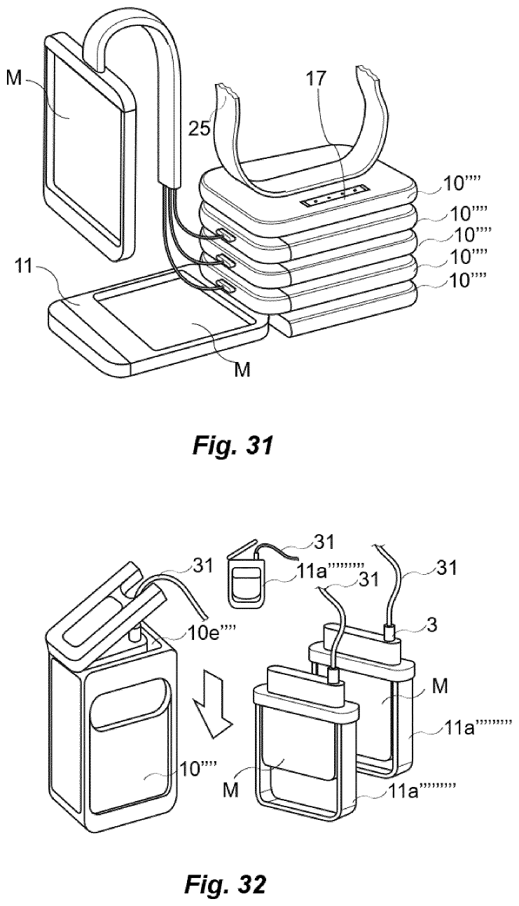

In another example, the cassette comprises two medicament containers. In one example, the cassette comprises two container carriers 10a″, 10b″, as shown in FIGS. 5-6 and FIG. 31. The two container carriers 10a″, 10b″ comprise two medicament containers respectively. The two container carriers 10a″, 10b″ comprise at least two fluid-tight chambers 11a′″, 11b′″; 11c′″ respectively. FIG. 5 and FIG. 31 shows one example where the medicament container comprises Ma, Mb, Mc the flexible bag positioned within the fluid-tight chambers 11a′″, 11b′″; 11c′″. In a preferred example, the two container carriers are stacked on one another, as shown in FIG. 31.

In one example where there is no cassette housing, the multiple cassettes are stacked on one another. Alternatively, or additionally, the cassette comprises a cassette housing, the two container carriers are stacked on one another within the cassette housing. FIG. 6 shows one example where the body M0a, M0b of the medicament container comprises the flexible tubes M0b positioned within the fluid-tight chambers 11a″″, 11b″″; 11c″″. Alternatively, the container carrier 10a″; 10′″″comprises at least two fluid-tight chambers 11a′″, 11b′″; 11a′″″″″, 10e′″″. In this example, the at least two fluid-tight chambers 11a′″, 11b′″ are configured to accommodate two flexible portions of the at least two bodies of the two medicament containers Ma, Mb respectively. In one example, as shown in FIG. 32, the two fluid-tight chambers 11a′″″″″, 10e′″″ can be formed by the container frame 11a′″″″″ and the interior chamber 10e′″″of the container 10′″″.

In one example, each medicament container is provided with a fluidically separate connection to the patient in a manner by which separate medications do not mix during administration unless desired at the patient site. For example, each medicament container is connected with its own medicament delivery member 23a′, 23b′, 23c′, 23d′.

In a preferred example, the cassette comprises more than two medicament containers, as shown in FIGS. 5-6, 13-14. FIG. 22, FIG. 24, and FIG. 32. In a preferred example, the multiple medicament containers are accommodated within the cassette with a combination of multiple container carriers and multiple fluid-tight chambers. For example, the cassette comprises three medicament containers and two container carriers. In this example, one container carrier comprises two fluid-tight chambers and is configured to accommodate two medicament containers respectively; the other one of the container carriers comprises one fluid-tight chamber configured to accommodate the third medicament container.

In a preferred example, a one-way valve 101 is arranged between two fluid-tight chambers 11a″, 11b″. The two fluid-tight chambers 11a″, 11b″ can be a part of one single container carrier or can be included in two container carriers respectively, as shown in FIG. 5 and FIG. 14.

In another preferred example, only one fluid-tight chamber 11a″ of one of the two fluid-tight chambers 11a″, 11b″ comprises the inlet 110 that is configured to be fluidly connected to the fluid pressure power source 21. In this example, the medicament container within the two medicament containers respectively can be sequentially delivered to the patient. For example, two medicament containers are configured to contain two different medicament and only the first predetermined medicament is fully delivered to the patient, the second predetermined medicament can be delivered to the patient.

For example, the one-way valve 101 between the two fluid-tight chambers 11a″, 11b″ is configured to open when a first fluid pressure threshold is reached. The valve 114 of the inlet 110′″ of the fluid-tight chamber that is configured to be fluidly connected to the fluid pressure power source 21 is configured to open when a second fluid pressure threshold is reached. The first fluid pressure threshold is equal to or greater than the second fluid pressure threshold. Alternatively, the first fluid pressure threshold can be lower than the second fluid pressure threshold, but greater than the resistant pressure from the tissue that the medicament delivery member is positioned within. In this example, the medicament delivery member is an injection needle or a soft cannula. The specific value of the resistant pressure is dependent on the target tissue, e.g., muscle or subcutaneous tissue.

In one example where the fluid-tight chamber comprises the release valve 115, the predetermined threshold is greater than the second fluid pressure threshold. In another preferred example, the predetermined threshold is greater than the first fluid pressure threshold.

In one example where the fluid-tight chamber comprises the inlet 110′ and outlet 113′, two one-way valves are positioned between the two fluid-tight chambers and are configured to open to the opposition direction. Alternatively, one multi-directions valve can be positioned between the two fluid-tight chambers. The multi-direction valve can be configured to open in different directions based on different pressure thresholds. The multi-directions valve can be a multi-directions valve, e.g., 2/2-way valve, 3/2-way valve, 5/2-way valve.

Some examples of the variant cassettes are explained in detail as follows.

In the first example, the cassette comprises the container carrier 10 with one fluid-tight chamber 11. More specifically, in this example, the container carrier 10 is the fluid-tight chamber 11, as shown in FIG. 1. In this example, the fluid-tight chamber 11 is made of rigid material. In the first example, the body MO of the medicament container M is the flexible bag. The flexible bag is partially or fully accommodated within the fluid-tight chamber 11 so that when the fluid flows into the fluid-tight chamber 11, the fluid will press on the flexible bag, and thereby expels at least a part of the contained medicament out of the fluid outlet M1 of the medicament container M. In this example, the fluid outlet M1 of the medicament container M is a part of the flexible bag, as shown in FIG. 8, and the fluid outlet M1 is configured to connect to a medicament delivery member of the medicament delivery device 2; 2; 2″ when the cassette is attached to the medicament delivery device 2; 2; 2″. More specifically, the fluid outlet M1 of the medicament container M is connected to an injection needle or a soft cannula that is configured to be placed under a skin of a patient. Additionally, the fluid-tight chamber 11 optionally comprises the release valve 115, as shown in FIG. 1. Furthermore, in a preferred example, the inlet 110 of the fluid-tight chamber 11 comprises a one-way valve 114, e.g., an umbrella valve or a Belleville spring valve or a ball valve. Alternatively, the inlet 110 of the fluid-tight chamber 11 comprises a multi-directions valve. Alternatively, in another example, the fluid-tight chamber 11 is made of flexible material, e.g., a secondary flexible bag 11″″, as shown in FIG. 12.

In the second example, the cassette comprises the container carrier 10 with one fluid-tight chamber. More specifically, in this example, the container carrier 10 is the fluid-tight chamber. In this example, the fluid-tight chamber is made of rigid material. In the second example, the body M0a, M0b of the medicament container M is a combination of the flexible bag M0a and the flexible tube M0b extending from the flexible bag M0a to a delivery tube outlet, as shown in FIGS. 2A-2B. In this example, the delivery tube M0b is flexible. In this example, the fluid outlet M1′ is the delivery tube outlet. The flexible bag M0a is partially or fully accommodated within the fluid-tight chamber so that when the fluid flows into the fluid-tight chamber, the fluid will press on the flexible bag, and thereby expels at least a part of the contained medicament out of the fluid outlet M1′ of the medicament container M. In this example, the fluid outlet M1′ of the medicament container M is configured to connect to a medicament delivery member of the medicament delivery device when the cassette is attached to the medicament delivery device. More specifically, the fluid outlet M1′ of the medicament container M is connected to an injection needle or a soft cannula that is configured to be placed under a skin of a patient. Additionally, the fluid-tight chamber optionally comprises the release valve 115, as shown in FIG. 1. Additionally, the inlet of the fluid-tight chamber optionally comprises a one-way tube valve, e.g., an umbrella valve or a Belleville spring valve or a ball valve. Additionally, the delivery tube M0b optionally comprises a tube valve, the tube valve is a one-way valve, e.g., an umbrella valve or a Belleville spring valve or a ball valve, the tube valve is configured to prevent any fluid from flowing back to the flexible bag M0a of the body M0a, M0b of the medicament container M through the delivery tube M0b. Alternatively, the inlet of the fluid-tight chamber comprises a multi-directions valve. Alternatively, in another example, the fluid-tight chamber is made of flexible material, e.g., a secondary flexible bag M0a11″″, as shown in FIG. 12.

In the third example, the cassette comprises the container carrier with one fluid-tight chamber. More specifically, in this example, the container carrier is the fluid-tight chamber. In this example, the fluid-tight chamber is made of rigid material. In the third example, the body of the medicament container is a combination of a glass cartridge and the delivery tube extending from the glass cartridge to a delivery tube outlet. In this example, the delivery tube is flexible. Alternatively, the cartridge can be made of rigid plastic material. In this example, the fluid outlet is the delivery tube outlet. In the third example, the delivery tube is partially accommodated within the fluid-tight chamber. In this example, the fluid-tight chamber comprises a tube inlet and a tube outlet. The delivery tube is configured to be positioned between the tube inlet and the tube outlet. Therefore, when the fluid flows into the fluid-tight chamber, the fluid will press on the delivery tube, and thereby expels at least a part of the contained medicament out of the fluid outlet of the medicament container. It should be noted that the delivery tube can be fully accommodate within the fluid-tight chamber. In the third example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Additionally, the delivery tube optionally comprises a tube valve. The tube valve is a one-way valve, e.g., an umbrella valve or a Belleville spring valve or a ball valve, the tube valve is configured to prevent any fluid from flowing back to the glass cartridge of the body of the medicament container through the delivery tube. Alternatively, the inlet of the fluid-tight chamber comprises a multi-directions valve. Alternatively, in another example, the fluid-tight chamber is made of flexible material, e.g., a secondary flexible bag 11″″.

In the fourth example, the cassette comprises the container carrier with one fluid-tight chamber. More specifically, in this example, the container carrier is the fluid-tight chamber. In this example, the fluid-tight chamber is made of rigid material. In this example, the body of the medicament container is a combination of a glass cartridge and the delivery tube extending from the glass cartridge to a delivery tube outlet. In this example, the delivery tube is flexible. Alternatively, the cartridge can be made of rigid plastic material. In this example, the fluid outlet is the delivery tube outlet. In this example, the delivery tube is partially accommodated within the fluid-tight chamber. In this example, the fluid-tight chamber comprises a tube inlet and a tube outlet. The delivery tube is configured to be positioned between the tube inlet and the tube outlet. In the fourth example, the delivery tube comprises two tube valves. The tube valves are a one-way valve, e.g., an umbrella valve or a Belleville spring valve or a ball valve, the tube valves are configured to prevent any fluid from flowing back to the flexible bag of the body of the medicament container through the delivery tube. More specifically, one of the two tube valves is close to the tube inlet of the fluid-tight chamber and the other one of the two tube valve is close to the tube outlet of the fluid-tight chamber. Therefore, when the fluid flows into the fluid-tight chamber, the fluid will press on the delivery tube, and thereby expels at least a part of the contained medicament out of the fluid outlet of the medicament container. It should be noted that the delivery tube can be fully accommodate within the fluid-tight chamber. In this example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve. Alternatively, in another example, the fluid-tight chamber is made of flexible material, e.g., a secondary flexible bag 11″″.

In the fifth example, the cassette comprises the container carrier with one fluid-tight chamber. More specifically, in this example, the container carrier is the fluid-tight chamber. In this example, the fluid-tight chamber is made of rigid material. In the fifth example, the fluid-tight chamber 11′ comprises the inlet 110′ and an outlet 113′, as shown in FIGS. 2A-2B. The inlet 110′ of the fluid-tight chamber 11′ is configured to be fluidly connected to the fluid pressure power source. The outlet 113′ of the fluid-tight chamber 11′ is configured to be connected to a vacuum device. In this example, the body of the medicament container is a combination of a glass cartridge and the delivery tube extending from the glass cartridge to a delivery tube outlet. In this example, the delivery tube is flexible. Alternatively, the cartridge can be made of rigid plastic material. In this example, the fluid outlet is the delivery tube outlet. In this example, the delivery tube is partially accommodated within the fluid-tight chamber. In this example, the fluid-tight chamber 11′ comprises a tube inlet 111′ and a tube outlet 112′, as shown in FIGS. 2A-2B. The delivery tube is configured to be positioned between the tube inlet 111′ and the tube outlet 112′. In the fifth example, the inlet 110′ of the fluid-tight chamber 11′ and the outlet 113′ of the fluid-tight chamber 11′ are positioned between the tube inlet 111′ of the fluid-tight chamber 11′ and the tube outlet 113′ of the fluid-tight chamber 11′. Therefore, when pressure within the fluid-tight chamber is reduced, a certain amount of medicament within the glass cartridge will be sucked into a portion of the delivery tube that is accommodated within the fluid-tight chamber. Afterward, when the fluid from the fluid pressure power source flows into the fluid-tight chamber, the fluid will press on the delivery tube, and thereby expels at least a part of the certain amount of medicament within the delivery tube out of the fluid outlet of the medicament container. It should be noted that the delivery tube can be fully accommodate within the fluid-tight chamber. In this example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Additionally, the delivery tube optionally comprises a tube valve. The tube valve is a one-way valve, e.g., an umbrella valve or a Belleville spring valve or a ball valve, the tube valve is configured to prevent any fluid from flowing back to the glass cartridge of the body of the medicament container through the delivery tube. Alternatively, the inlet of the fluid-tight chamber comprises a multi-directions valve. Alternatively, in another example, the fluid-tight chamber is made of flexible material, e.g., a secondary flexible bag 11″″, as shown in FIG. 12.

In the sixth example, the cassette comprises the container carrier with one fluid-tight chamber. More specifically, in this example, the container carrier is the fluid-tight chamber. In this example, the fluid-tight chamber is made of rigid material. In the sixth example, the fluid-tight chamber comprises the inlet and an outlet. In the sixth example, the inlet of the fluid-tight chamber and the inlet of the fluid-tight chamber is a same orifice 110″, 113″ of the fluid-tight chamber, as shown in FIGS. 3A-B. In the sixth example, the orifice 110″, 113″ of the fluid-tight chamber is connected to a device that can provide both a vacuum and an output fluid. In this example, the body of the medicament container is a combination of a glass cartridge and the delivery tube extending from the glass cartridge to a delivery tube outlet. In this example, the delivery tube is flexible. Alternatively, the cartridge can be made of rigid plastic material. In this example, the fluid outlet is the delivery tube outlet. In the sixth example, the delivery tube is partially accommodated within the fluid-tight chamber. In this example, the fluid-tight chamber comprises a tube inlet and a tube outlet. The delivery tube is configured to be positioned between the tube inlet and the tube outlet. In the example, the orifice 110″, 113″ of the fluid-tight chamber is positioned between the tube inlet of the fluid-tight chamber and the tube outlet of the fluid-tight chamber. Therefore, when pressure within the fluid-tight chamber is reduced, a certain amount of medicament within the glass cartridge will be sucked into a portion of the delivery tube that is accommodated within the fluid-tight chamber. Afterward, when the output fluid flows into the fluid-tight chamber, the fluid will press on the delivery tube, and thereby expels at least a part of the certain amount of medicament within the delivery tube out of the fluid outlet of the medicament container. It should be noted that the delivery tube can be fully accommodate within the fluid-tight chamber. In the sixth example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115, as shown in FIG. 1. Additionally, the inlet of the fluid-tight chamber optionally comprises a one-way tube valve, e.g., an umbrella valve or a Belleville spring valve or a ball valve. Additionally, the delivery tube optionally comprises a tube valve. The tube valve is a one-way valve, e.g., an umbrella valve or a Belleville spring valve or a ball valve, the tube valve is configured to prevent any fluid from flowing back to the glass cartridge of the body of the medicament container through the delivery tube. Alternatively, the inlet of the fluid-tight chamber comprises a multi-directions valve. Alternatively, in another example, the fluid-tight chamber is made of flexible material, e.g., a secondary flexible bag 11″″, as shown in FIG. 12.

In the seventh example, the cassette comprises the container carrier with one fluid-tight chamber. More specifically, in this example, the container carrier is the fluid-tight chamber. In this example, the fluid-tight chamber is made of rigid material. In the seventh example, the fluid-tight chamber comprises the inlet and an outlet. The inlet of the fluid-tight chamber is configured to be fluidly connected to the fluid pressure power source. The outlet of the fluid-tight chamber is configured to be connected to a vacuum device. In this example, the body of the medicament container is a combination of a glass cartridge and the delivery tube extending from the glass cartridge to a delivery tube outlet. In this example, the delivery tube is flexible. Alternatively, the cartridge can be made of rigid plastic material. In this example, the fluid outlet is the delivery tube outlet. In this example, the delivery tube is partially accommodated within the fluid-tight chamber. In this example, the fluid-tight chamber comprises a tube inlet and a tube outlet. The delivery tube is configured to be positioned between the tube inlet and the tube outlet. In the seventh example, the delivery tube comprises two tube valves. The tube valves are a one-way valve, e.g., an umbrella valve or a Belleville spring valve or a ball valve, the tube valves are configured to prevent any fluid from flowing back to the flexible bag of the body of the medicament container through the delivery tube. More specifically, one of the two tube valves is close to the tube inlet of the fluid-tight chamber and the other one of the two tube valve is close to the tube outlet of the fluid-tight chamber. In the seventh example, the inlet of the fluid-tight chamber and the outlet of the fluid-tight chamber are positioned between the tube inlet of the fluid-tight chamber and the tube outlet of the fluid-tight chamber. Therefore, when pressure within the fluid-tight chamber is reduced, a certain amount of medicament within the glass cartridge will be sucked into a portion of the delivery tube that is accommodated within the fluid-tight chamber. Afterward, when the fluid from the fluid pressure power source flows into the fluid-tight chamber, the fluid will press on the delivery tube, and thereby expels at least a part of the certain amount of medicament within the delivery tube out of the fluid outlet of the medicament container. It should be noted that the delivery tube can be fully accommodate within the fluid-tight chamber. In this example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115, as shown in FIG. 1. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve. Alternatively, in another example, the fluid-tight chamber is made of flexible material, e.g., a secondary flexible bag 11″″, as shown in FIG. 12.

In the eighth example, the cassette comprises the container carrier with one fluid-tight chamber. More specifically, in this example, the container carrier is the fluid-tight chamber. In this example, the fluid-tight chamber is made of rigid material. In the eighth example, the fluid-tight chamber comprises the inlet and an outlet. In this example, the inlet of the fluid-tight chamber and the inlet of the fluid-tight chamber is a same orifice 110″, 113″ of the fluid-tight chamber, as shown in FIGS. 3A-3B. In this example, the orifice 110″, 113″ of the fluid-tight chamber is connected to a device that can provide both a vacuum and an output fluid. In this example, the body of the medicament container is a combination of a glass cartridge and the delivery tube extending from the glass cartridge to a delivery tube outlet. In this example, the delivery tube is flexible. Alternatively, the cartridge can be made of rigid plastic material. In this example, the fluid outlet is the delivery tube outlet. In this example, the delivery tube is partially accommodated within the fluid-tight chamber. In this example, the fluid-tight chamber comprises a tube inlet and a tube outlet. The delivery tube is configured to be positioned between the tube inlet and the tube outlet. In this example, the orifice 110″, 113″ of the fluid-tight chamber is positioned between the tube inlet of the fluid-tight chamber and the tube outlet of the fluid-tight chamber. In the eighth example, the delivery tube comprises two tube valves. The tube valves are a one-way valve, e.g., an umbrella valve or a Belleville spring valve or a ball valve, the tube valves are configured to prevent any fluid from flowing back to the flexible bag of the body of the medicament container through the delivery tube. More specifically, one of the two tube valves is close to the tube inlet of the fluid-tight chamber and the other one of the two tube valve is close to the tube outlet of the fluid-tight chamber. In this example, the inlet of the fluid-tight chamber and the outlet of the fluid-tight chamber are positioned between the tube inlet of the fluid-tight chamber and the tube outlet of the fluid-tight chamber. Therefore, when pressure within the fluid-tight chamber is reduced, a certain amount of medicament within the glass cartridge will be sucked into a portion of the delivery tube that is accommodated within the fluid-tight chamber. Afterward, when the output fluid flows into the fluid-tight chamber, the fluid will press on the delivery tube, and thereby expels at least a part of the certain amount of medicament within the delivery tube out of the fluid outlet of the medicament container. It should be noted that the delivery tube can be fully accommodate within the fluid-tight chamber. In this example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115, as shown in FIG. 1. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve. Alternatively, in another example, the fluid-tight chamber is made of flexible material, e.g., a secondary flexible bag 11″″, as shown in FIG. 12.

In the ninth example, the cassette comprises the container carrier with one fluid-tight chamber 11′. More specifically, in this example, the container carrier is the fluid-tight chamber 11′. In this example, the fluid-tight chamber 11′ is made of rigid material. In the ninth example, the body M0a, M0b of the medicament container is a combination of the flexible bag M0a and the delivery tube M0b extending from the flexible bag M0a to a delivery tube outlet, as shown in FIGS. 2A-2B. In this example, the delivery tube M0b is flexible. In this example, the fluid outlet M1′ is the delivery tube outlet. In the ninth example, the delivery tube M0b is partially accommodated within the fluid-tight chamber 11′. In this example, the fluid-tight chamber 11′ comprises a tube inlet and a tube outlet. The delivery tube is configured to be positioned between the tube inlet 111′ and the tube outlet 112′. Therefore, when the fluid flows into the fluid-tight chamber, the fluid will press on the delivery tube, and thereby expels at least a part of the contained medicament out of the fluid outlet of the medicament container. It should be noted that the delivery tube can be fully accommodate within the fluid-tight chamber. In the ninth example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115, as shown in FIG. 1. Additionally, the inlet of the fluid-tight chamber optionally comprises a one-way tube valve, e.g., an umbrella valve or a Belleville spring valve or a ball valve. Additionally, the delivery tube optionally comprises a tube valve. The tube valve is a one-way valve, e.g., an umbrella valve or a Belleville spring valve or a ball valve, the tube valve is configured to prevent any fluid from flowing back to the flexible bag of the body of the medicament container through the delivery tube. Alternatively, the inlet of the fluid-tight chamber comprises a multi-directions valve. Alternatively, in another example, the fluid-tight chamber is made of flexible material, e.g., a secondary flexible bag 11″″, as shown in FIG. 12.

In the tenth example, the cassette comprises the container carrier with one fluid-tight chamber 11′. More specifically, in this example, the container carrier is the fluid-tight chamber 11′. In this example, the fluid-tight chamber is made of rigid material. In this example, the body M0a, M0b of the medicament container is a combination of the flexible bag M0a and the delivery tube M0b extending from the flexible bag M0a to a delivery tube outlet, as shown in FIGS. 2A-2B. In this example, the delivery tube is flexible. In this example, the fluid outlet M1′ is the delivery tube outlet. In this example, the delivery tube is partially accommodated within the fluid-tight chamber. In this example, the fluid-tight chamber comprises a tube inlet 111′ and a tube outlet 112′. The delivery tube is configured to be positioned between the tube inlet and the tube outlet. In the tenth example, the delivery tube comprises two tube valves M2, M2′. The tube valves M2, M2′ are a one-way valve, e.g., an umbrella valve or a Belleville spring valve or a ball valve, the tube valves M2, M2′ are configured to prevent any fluid from flowing back to the flexible bag of the body of the medicament container through the delivery tube. More specifically, one of the two tube valves M2, M2′ is close to the tube inlet 111′ of the fluid-tight chamber 11′ and the other one of the two tube valves M2, M2′ is close to the tube outlet 112′ of the fluid-tight chamber 11′. Therefore, when the fluid flows into the fluid-tight chamber, the fluid will press on the delivery tube, and thereby expels at least a part of the contained medicament out of the fluid outlet of the medicament container. It should be noted that the delivery tube can be fully accommodate within the fluid-tight chamber. In this example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115, as shown in FIG. 1. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve. Alternatively, in another example, the fluid-tight chamber is made of flexible material, e.g., a secondary flexible bag 11″″, as shown in FIG. 12.

In the eleventh example, the cassette comprises the container carrier with one fluid-tight chamber. More specifically, in this example, the container carrier is the fluid-tight chamber. In this example, the fluid-tight chamber is made of rigid material. In the eleventh example, as shown in FIGS. 2A-2B, the fluid-tight chamber comprises the inlet 111′ and an outlet 113′. The inlet 110′ of the fluid-tight chamber 11′ is configured to be fluidly connected to the fluid pressure power source. The outlet 113′ of the fluid-tight chamber 11′ is configured to be connected to a vacuum device. In this example, the body M0a, M0b of the medicament container is a combination of the flexible bag M0a and the delivery tube M0b extending from the flexible bag to a delivery tube outlet. In this example, the delivery tube is flexible. In this example, the fluid outlet M1′ is the delivery tube outlet. In this example, the delivery tube is partially accommodated within the fluid-tight chamber. In this example, the fluid-tight chamber 11′ comprises a tube inlet 111′ and a tube outlet 112′. The delivery tube M0b is configured to be positioned between the tube inlet 111′ and the tube outlet 112′. In the eleventh example, the inlet 110′ of the fluid-tight chamber 11′ and the outlet 113′ of the fluid-tight chamber 11′ are positioned between the tube inlet 111′ of the fluid-tight chamber 11′ and the tube outlet 112′ of the fluid-tight chamber 11′. Therefore, when pressure within the fluid-tight chamber 11′ is reduced, a certain amount of medicament within the flexible bag M0a will be sucked into a portion of the delivery tube M0b that is accommodated within the fluid-tight chamber 11′. Afterward, when the fluid from the fluid pressure power source flows into the fluid-tight chamber 11′, the fluid will press on the delivery tube M0b, and thereby expels at least a part of the certain amount of medicament within the delivery tube M0b out of the fluid outlet of the medicament container. It should be noted that the delivery tube can be fully accommodate within the fluid-tight chamber. In this example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115, as shown in FIG. 1. Additionally, the inlet of the fluid-tight chamber optionally comprises a one-way tube valve, e.g., an umbrella valve or a Belleville spring valve or a ball valve. Additionally, the delivery tube optionally comprises a tube valve. The tube valve is a one-way valve, e.g., an umbrella valve or a Belleville spring valve or a ball valve, the tube valve is configured to prevent any fluid from flowing back to the flexible bag of the body of the medicament container through the delivery tube. Alternatively, the inlet of the fluid-tight chamber comprises a multi-directions valve. Alternatively, in another example, the fluid-tight chamber is made of flexible material, e.g., a secondary flexible bag 11″″, as shown in FIG. 12.

In the twelfth example, the cassette comprises the container carrier with one fluid-tight chamber 11′. More specifically, in this example, the container carrier is the fluid-tight chamber 11′. In this example, the fluid-tight chamber 11′ is made of rigid material. In the twelfth example, as shown in FIGS. 3A-3B, the fluid-tight chamber 11′ comprises the inlet and an outlet. In the twelfth example, the inlet 110″ of the fluid-tight chamber 11′ and the outlet 113″ of the fluid-tight chamber 11′ is a same orifice of the fluid-tight chamber 11′. In the twelfth example, the orifice 110″, 113″ of the fluid-tight chamber 11′ is connected to a device that can provide both a vacuum and an output fluid. In this example, the body of the medicament container is a combination of the flexible bag and the delivery tube extending from the flexible bag to a delivery tube outlet. In this example, the delivery tube is flexible. In this example, the fluid outlet is the delivery tube outlet. In the twelfth example, the delivery tube is partially accommodated within the fluid-tight chamber 11′. In this example, the fluid-tight chamber 11′ comprises a tube inlet and a tube outlet. The delivery tube is configured to be positioned between the tube inlet and the tube outlet. In the example, the orifice 110″, 113″ of the fluid-tight chamber 11′ is positioned between the tube inlet 110″ of the fluid-tight chamber 11′ and the tube outlet of the fluid-tight chamber 11′. Therefore, when pressure within the fluid-tight chamber 11′ is reduced by, a certain amount of medicament within the flexible bag will be sucked into a portion of the delivery tube that is accommodated within the fluid-tight chamber 11′. Afterward, when the output fluid flows into the fluid-tight chamber 11′, the fluid will press on the delivery tube, and thereby expels at least a part of the certain amount of medicament within the delivery tube out of the fluid outlet of the medicament container. It should be noted that the delivery tube can be fully accommodate within the fluid-tight chamber 11′. In the twelfth example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber 11′ optionally comprises the release valve 115, as shown in FIG. 1. Additionally, the inlet 110″ of the fluid-tight chamber 11′ optionally comprises a one-way tube valve, e.g., an umbrella valve or a Belleville spring valve or a ball valve. Additionally, the delivery tube optionally comprises a tube valve. The tube valve is a one-way valve, e.g., an umbrella valve or a Belleville spring valve or a ball valve, the tube valve is configured to prevent any fluid from flowing back to the flexible bag of the body of the medicament container through the delivery tube. Alternatively, the inlet 110″ of the fluid-tight chamber 11′ comprises a multi-directions valve. Alternatively, in another example, the fluid-tight chamber 11′ is made of flexible material, e.g., a secondary flexible bag 11″″, as shown in FIG. 12.

In the thirteenth example, the cassette comprises the container carrier with one fluid-tight chamber. More specifically, in this example, the container carrier is the fluid-tight chamber. In this example, the fluid-tight chamber is made of rigid material. In the thirteenth example, the fluid-tight chamber comprises the inlet and an outlet. The inlet of the fluid-tight chamber is configured to be fluidly connected to the fluid pressure power source. The outlet of the fluid-tight chamber is configured to be connected to a vacuum device. In this example, the body of the medicament container is a combination of the flexible bag and the delivery tube extending from the flexible bag to a delivery tube outlet. In this example, the delivery tube is flexible. In this example, the fluid outlet is the delivery tube outlet. In this example, the delivery tube is partially accommodated within the fluid-tight chamber. In this example, the fluid-tight chamber comprises a tube inlet and a tube outlet. The delivery tube is configured to be positioned between the tube inlet and the tube outlet. In the thirteenth example, the delivery tube comprises two tube valves. The tube valves are a one-way valve, e.g., an umbrella valve or a Belleville spring valve or a ball valve, the tube valves are configured to prevent any fluid from flowing back to the flexible bag of the body of the medicament container through the delivery tube. More specifically, one of the two tube valves is close to the tube inlet of the fluid-tight chamber and the other one of the two tube valve is close to the tube outlet of the fluid-tight chamber. In the thirteenth example, the inlet of the fluid-tight chamber and the outlet of the fluid-tight chamber are positioned between the tube inlet of the fluid-tight chamber and the tube outlet of the fluid-tight chamber. Therefore, when pressure within the fluid-tight chamber is reduced, a certain amount of medicament within the flexible bag will be sucked into a portion of the delivery tube that is accommodated within the fluid-tight chamber. Afterward, when the fluid from the fluid pressure power source flows into the fluid-tight chamber, the fluid will press on the delivery tube, and thereby expels at least a part of the certain amount of medicament within the delivery tube out of the fluid outlet of the medicament container. It should be noted that the delivery tube can be fully accommodate within the fluid-tight chamber. In this example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve. Alternatively, in another example, the fluid-tight chamber is made of flexible material, e.g., a secondary flexible bag 11″″.

In the fourteenth example, the cassette comprises the container carrier with one fluid-tight chamber. More specifically, in this example, the container carrier is the fluid-tight chamber. In this example, the fluid-tight chamber is made of rigid material. In the fourteenth example, the fluid-tight chamber comprises the inlet and an outlet. In this example, the inlet of the fluid-tight chamber and the outlet of the fluid-tight chamber is a same orifice 110″, 113″ of the fluid-tight chamber, as shown in FIGS. 3A-3B. In this example, the orifice 110″, 113″ of the fluid-tight chamber is connected to a device that can provide both a vacuum and an output fluid. In this example, the body of the medicament container is a combination of the flexible bag and the delivery tube extending from the flexible bag to a delivery tube outlet. In this example, the delivery tube is flexible. In this example, the fluid outlet is the delivery tube outlet. In this example, the delivery tube is partially accommodated within the fluid-tight chamber. In this example, the fluid-tight chamber comprises a tube inlet and a tube outlet. The delivery tube is configured to be positioned between the tube inlet and the tube outlet. In this example, the orifice 110″, 113″ of the fluid-tight chamber is positioned between the tube inlet of the fluid-tight chamber and the tube outlet of the fluid-tight chamber. In the fourteenth example, the delivery tube comprises two tube valves. The tube valves are a one-way valve, e.g., an umbrella valve or a Belleville spring valve or a ball valve, the tube valves are configured to prevent any fluid from flowing back to the flexible bag of the body of the medicament container through the delivery tube. More specifically, one of the two tube valves is close to the tube inlet of the fluid-tight chamber and the other one of the two tube valve is close to the tube outlet of the fluid-tight chamber. In this example, the inlet of the fluid-tight chamber and the outlet of the fluid-tight chamber are positioned between the tube inlet of the fluid-tight chamber and the tube outlet of the fluid-tight chamber. Therefore, when pressure within the fluid-tight chamber is reduced, a certain amount of medicament within the flexible bag will be sucked into a portion of the delivery tube that is accommodated within the fluid-tight chamber. Afterward, when the output fluid flows into the fluid-tight chamber, the fluid will press on the delivery tube, and thereby expels at least a part of the certain amount of medicament within the delivery tube out of the fluid outlet of the medicament container. It should be noted that the delivery tube can be fully accommodate within the fluid-tight chamber. In this example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve. Alternatively, in another example, the fluid-tight chamber is made of flexible material, e.g., a secondary flexible bag 11″″.

In the fifteenth example, the cassette comprises the container carrier with multiple fluid-tight chambers. In this example, each fluid-tight chamber is made of rigid material. In the fifteenth example, the container carrier is configured to accommodate multiple medicament containers within the multiple fluid-tight chambers respectively. In this example, each of the multiple medicament containers are identical. Alternatively, each of the multiple medicament containers has the same shape to one another but is sized differently to one another. Alternatively, at least two of the multiple medicament containers are geometrically different from one another. In the fifteenth example, each body of each of the multiple medicament container is the flexible bag. Each flexible bag is partially or fully accommodated within one of the multiple fluid-tight chambers so that when the fluid flows into the fluid-tight chamber, the fluid will press on the flexible bag, and thereby expels at least a part of the contained medicament out of the fluid outlet of the medicament container. In this example, the fluid outlet of at least one of the multiple medicament containers is a part of the flexible bag, and the fluid outlet of at least one of the multiple medicament containers is configured to connect to a medicament delivery member of the medicament delivery device when the cassette is attached to the medicament delivery device. More specifically, the fluid outlet of at least one of the multiple medicament containers is connected to an injection needle or a soft cannula that is configured to be placed under a skin of a patient. Alternatively, the fluid outlet of each of the multiple medicament containers is a part of each flexible bag, and the fluid outlet of each of the multiple medicament containers is configured to connect to one independent medicament delivery member of the medicament delivery device when the cassette is attached to the medicament delivery device. More specifically, the fluid outlet of each of the multiple medicament containers is connected to one injection needle and/or one soft cannula that is configured to be placed under a skin of a patient. Additionally, at least one of the multiple fluid-tight chambers, in this example, comprises the release valve. Furthermore, in a preferred example, the inlet of at least one of the multiple fluid-tight chambers comprises a one-way valve, e.g., an umbrella valve or a Belleville spring valve or a ball valve. Alternatively, the inlet of at least one of the multiple fluid-tight chambers comprises a multi-directions valve. Alternatively, in another example, at least one of the multiple fluid-tight chambers is made of flexible material, e.g., a secondary flexible bag 11″″. Additionally, at least one of the multiple fluid-tight chambers optionally comprises the release valve 115.

In the sixteenth example, the cassette comprises the container carrier with multiple fluid-tight chambers. In this example, each fluid-tight chamber is made of rigid material. In the sixteenth example, the container carrier is configured to accommodate multiple medicament containers within the multiple fluid-tight chambers respectively. In this example, each of the multiple medicament containers are identical. Alternatively, each of the multiple medicament containers has the same shape to one another but is sized differently to one another. Alternatively, at least two of the multiple medicament containers are geometrically different from one another. In the sixteenth example, each body of each of the multiple medicament container is the flexible bag. Each flexible bag is partially or fully accommodated within one of the multiple fluid-tight chambers. In this example, only one of the multiple fluid-tight chambers comprises the inlet configured to be fluidly connected to the fluid pressure power source. In this example, a one-way valve is located between each two fluid-tight chambers of the multiple chambers. Therefore, when the fluid flows firstly into the fluid-tight chamber that comprises the inlet, and then sequentially flows into each of the rest of the multiple fluid-tight chambers via each one-way valve between each two fluid-tight chambers of the multiple chambers. Thus, the fluid will sequentially press on each of flexible bag within different the multiple fluid-tight chambers, and thereby sequentially expels at least a part of the contained medicament out of the fluid outlet of each the medicament containers. In this example, the fluid outlet of at least one of the multiple medicament containers is a part of the flexible bag, and the fluid outlet of at least one of the multiple medicament containers is configured to connect to a medicament delivery member of the medicament delivery device when the cassette is attached to the medicament delivery device. More specifically, the fluid outlet of at least one of the multiple medicament containers is connected to an injection needle or a soft cannula that is configured to be placed under a skin of a patient. Alternatively, the fluid outlet of each of the multiple medicament containers is a part of each flexible bag, and the fluid outlet of each of the multiple medicament containers is configured to connect to one independent medicament delivery member of the medicament delivery device when the cassette is attached to the medicament delivery device. More specifically, the fluid outlet of each of the multiple medicament containers is connected to one injection needle and/or one soft cannula that is configured to be placed under a skin of a patient. Additionally, at least one of the multiple fluid-tight chambers, in this example, comprises the release valve. Furthermore, in a preferred example, the only one inlet of the multiple fluid-tight chambers comprises a one-way valve, e.g., an umbrella valve or a Belleville spring valve or a ball valve. Alternatively, the only one inlet of the multiple fluid-tight chambers comprises a multi-directions valve. Alternatively, in another example, at least one of the multiple fluid-tight chambers is made of flexible material, e.g., a secondary flexible bag 11″″. Additionally, at least one of the multiple fluid-tight chambers optionally comprises the release valve 115.

In the seventeenth example, the cassette comprises the container carrier with multiple fluid-tight chambers. In this example, each fluid-tight chamber is made of rigid material. In the seventeenth example, the container carrier is configured to accommodate multiple medicament containers within the multiple fluid-tight chambers respectively. In this example, each of the multiple medicament containers are identical. Alternatively, each of the multiple medicament containers has the same shape to one another but is sized differently to one another. Alternatively, at least two of the multiple medicament containers are geometrically different from one another. In the seventeenth example, each body of each of the multiple medicament container is a combination of the flexible bag and the flexible tube extending from the flexible bag to a delivery tube outlet. In this example, the delivery tube is flexible. In this example, the fluid outlet is the delivery tube outlet. Each flexible tube is partially or fully accommodated within one of the multiple fluid-tight chambers so that when the fluid flows into the fluid-tight chamber, the fluid will press on the flexible tube, and thereby expels at least a part of the contained medicament out of the fluid outlet of the medicament container. In this example, the fluid outlet of at least one of the multiple medicament containers is configured to connect to a medicament delivery member of the medicament delivery device when the cassette is attached to the medicament delivery device. More specifically, the fluid outlet of at least one of the multiple medicament containers is connected to an injection needle or a soft cannula that is configured to be placed under a skin of a patient. Alternatively, the fluid outlet of each of the multiple medicament containers is a part of each flexible bag, and the fluid outlet of each of the multiple medicament containers is configured to connect to one independent medicament delivery member of the medicament delivery device when the cassette is attached to the medicament delivery device. More specifically, the fluid outlet of each of the multiple medicament containers is connected to one injection needle and/or one soft cannula that is configured to be placed under a skin of a patient. Additionally, at least one of the multiple fluid-tight chambers, in this example, comprises the release valve. Furthermore, in a preferred example, the inlet of at least one of the multiple fluid-tight chambers comprises a one-way valve, e.g., an umbrella valve or a Belleville spring valve or a ball valve. Additionally, the delivery tube optionally comprises a tube valve, the tube valve is a one-way valve, e.g., an umbrella valve or a Belleville spring valve or a ball valve, the tube valve is configured to prevent any fluid from flowing back to the flexible bag of the body of the medicament container through the delivery tube. Alternatively, the inlet of at least one of the multiple fluid-tight chambers comprises a multi-directions valve. Alternatively, in another example, at least one of the multiple fluid-tight chambers is made of flexible material, e.g., a secondary flexible bag 11″″. Additionally, the at least one of the multiple fluid-tight chambers optionally comprises the release valve 115.

In the eighteenth example, the cassette comprises the container carrier with multiple fluid-tight chambers. In this example, each fluid-tight chamber is made of rigid material. In the eighteenth example, the container carrier is configured to accommodate multiple medicament containers within the multiple fluid-tight chambers respectively. In this example, each of the multiple medicament containers are identical. Alternatively, each of the multiple medicament containers has the same shape to one another but is sized differently to one another. Alternatively, at least two of the multiple medicament containers are geometrically different from one another. In the eighteenth example, each body of each of the multiple medicament container is a combination of a cartridge and the flexible tube extending from the cartridge to a delivery tube outlet. The cartridge is made of glass or plastic. In this example, the delivery tube is flexible. In this example, the fluid outlet is the delivery tube outlet. Each flexible tube is partially or fully accommodated within one of the multiple fluid-tight chambers so that when the fluid flows into the fluid-tight chamber, the fluid will press on the flexible tube, and thereby expels at least a part of the contained medicament out of the fluid outlet of the medicament container. In this example, the fluid outlet of at least one of the multiple medicament containers is configured to connect to a medicament delivery member of the medicament delivery device when the cassette is attached to the medicament delivery device. More specifically, the fluid outlet of at least one of the multiple medicament containers is connected to an injection needle or a soft cannula that is configured to be placed under a skin of a patient. Alternatively, the fluid outlet of each of the multiple medicament containers is a part of each flexible bag, and the fluid outlet of each of the multiple medicament containers is configured to connect to one independent medicament delivery member of the medicament delivery device when the cassette is attached to the medicament delivery device. More specifically, the fluid outlet of each of the multiple medicament containers is connected to one injection needle and/or one soft cannula that is configured to be placed under a skin of a patient. Additionally, at least one of the multiple fluid-tight chambers, in this example, comprises the release valve. Furthermore, in a preferred example, the inlet of at least one of the multiple fluid-tight chambers comprises a one-way valve, e.g., an umbrella valve or a Belleville spring valve or a ball valve. Additionally, the delivery tube optionally comprises a tube valve, the tube valve is a one-way valve, e.g., an umbrella valve or a Belleville spring valve or a ball valve, the tube valve is configured to prevent any fluid from flowing back to the cartridge of the body of the medicament container through the delivery tube. Alternatively, the inlet of at least one of the multiple fluid-tight chambers comprises a multi-directions valve. Alternatively, in another example, at least one of the multiple fluid-tight chambers is made of flexible material, e.g., a secondary flexible bag 11″″. Additionally, at least one of the multiple fluid-tight chambers optionally comprises the release valve 115.

In the nineteenth example, the cassette comprises the container carrier with one fluid-tight chamber. More specifically, in this example, the fluid-tight chamber is inflatable. In this example the container carrier is made of rigid material. In this example, the body of the medicament container is the flexible bag. In this example, the flexible bag and the fluid-tight chamber are both accommodated within the container carrier. The flexible bag is adjacent to the fluid-tight chamber so that when the fluid flows into the fluid-tight chamber, the fluid-tight chamber is inflated, therefore, the fluid-tight chamber will press on the flexible bag, and thereby expels at least a part of the contained medicament out of the fluid outlet of the medicament container. In this example, the fluid outlet of the medicament container is a part of the flexible bag, and the fluid outlet is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115. Furthermore, in a preferred example, the inlet of the fluid-tight chamber comprises a one-way valve, e.g., an umbrella valve or a Belleville spring valve or a ball valve. Alternatively, the inlet of the fluid-tight chamber comprises a multi-directions valve.

In the twentieth example, the cassette comprises the container carrier 10′ with one fluid-tight chamber 11″. More specifically, in this example, the fluid-tight chamber 11″ is inflatable. In this example the container carrier 10′ is made of rigid material. In this example, the body of the medicament container is a combination of the flexible bag and the delivery tube extending from the flexible bag to a delivery tube outlet. In this example, the flexible bag and the fluid-tight chamber 11″ are both accommodated within the container carrier 10′. The flexible bag is adjacent to the fluid-tight chamber 11″ so that when the fluid flows into the fluid-tight chamber 11″, the fluid-tight chamber 11″ is inflated, therefore, the fluid-tight chamber 11″ will press on the flexible bag, and thereby expels at least a part of the contained medicament out of the fluid outlet of the medicament container, as shown in FIG. 4. In this example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber 11″ optionally comprises the release valve 115. Additionally, the inlet of the fluid-tight chamber 11″ optionally comprises a one-way tube valve, e.g., an umbrella valve or a Belleville spring valve or a ball valve. Additionally, the delivery tube optionally comprises a tube valve, the tube valve is a one-way valve, e.g., an umbrella valve or a Belleville spring valve or a ball valve, the tube valve is configured to prevent any fluid from flowing back to the flexible bag of the body of the medicament container through the delivery tube. Alternatively, the inlet of the fluid-tight chamber 11″ comprises a multi-directions valve.

In the twenty-first example, the cassette comprises the container carrier with one fluid-tight chamber. More specifically, in this example, the fluid-tight chamber is inflatable. In this example the container carrier is made of rigid material. In the twenty-first example, the body of the medicament container is a combination of a glass cartridge and the delivery tube extending from the glass cartridge to a delivery tube outlet. In this example, the delivery tube is flexible. Alternatively, the cartridge can be made of rigid plastic material. In this example, the fluid outlet is the delivery tube outlet. In this example, the flexible tube and the fluid-tight chamber are both at least partially accommodated within the container carrier. The flexible tube is adjacent to the fluid-tight chamber so that when the fluid flows into the fluid-tight chamber, the fluid-tight chamber is inflated, therefore, the fluid-tight chamber will press on the flexible tube, and thereby expels at least a part of the contained medicament out of the fluid outlet of the medicament container. In the twenty-first example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Additionally, the delivery tube optionally comprises a tube valve. The tube valve is a one-way valve, e.g., an umbrella valve or a Belleville spring valve or a ball valve, the tube valve is configured to prevent any fluid from flowing back to the glass cartridge of the body of the medicament container through the delivery tube. Alternatively, the inlet of the fluid-tight chamber comprises a multi-directions valve.

In the twenty-second example, the cassette comprises the container carrier with one fluid-tight chamber. More specifically, in this example, the fluid-tight chamber is inflatable. In this example the container carrier is made of rigid material. In the twenty-second example, the body of the medicament container is a combination of the flexible bag and the delivery tube extending from the flexible bag to a delivery tube outlet. In this example, the delivery tube is flexible. In this example, the fluid outlet is the delivery tube outlet. In this example, the flexible tube and the fluid-tight chamber are both at least partially accommodated within the container carrier. The flexible tube is adjacent to the fluid-tight chamber so that when the fluid flows into the fluid-tight chamber, the fluid-tight chamber is inflated, therefore, the fluid-tight chamber will press on the flexible tube, and thereby expels at least a part of the contained medicament out of the fluid outlet of the medicament container. In the twenty-second example, the fluid outlet of the medicament container is configured to connect to a medicament delivery member of the medicament delivery device when the cassette is attached to the medicament delivery device. More specifically, the fluid outlet of the medicament container is connected to an injection needle or a soft cannula that is configured to be placed under a skin of a patient. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Additionally, the delivery tube optionally comprises a tube valve. The tube valve is a one-way valve, e.g., an umbrella valve or a Belleville spring valve or a ball valve, the tube valve is configured to prevent any fluid from flowing back to the flexible bag of the body of the medicament container through the delivery tube. Alternatively, the inlet of the fluid-tight chamber comprises a multi-directions valve.

In the twenty-third example, the cassette comprises the container carrier 10″″ with one fluid-tight chamber. In this example, the fluid-tight chamber 11 is made of rigid material. In this example, the fluid-tight chamber 11 is configured to be placed within the container carrier 10. In this example, the container carrier 10″″ comprises a connecting port 17 configured to be releasably attached to either the outlet of the fluid pressure power source of the reusable body of the medicament delivery device or another connecting port of another container carrier 10″″. In this example, each container carrier 10″″ is configured to stacked on one another, as shown in FIG. 31. In a preferred example, the body of the medicament container is a flexible bag. In a preferred example, the fluid outlet of the medicament container M is configured to be fluidly connected to a tube set 3. Preferably, the medicament container is configured to be attached to the tube set 3. The tube set 3 comprises the delivery tube 31. Preferably, the fluid outlet of the medicament container is configured to be connected to a medicament delivery member via the delivery tube. In this example, the fluid outlet of the medicament container is configured to be attached at one end of the delivery tube; and the medicament delivery member is configured to be attached at the other end of the delivery tube. Preferably, the tube set comprises the piercing member 32 configured to penetrate the fluid outlet of the medicament container M to establish fluid communication between the delivery tube 31 and the medicament container M, as shown in FIG. 34C. In this example, the medicament container can be completely sealed before use. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve.

In the twenty-fourth, the cassette comprises the container carrier 10″″ with at least two fluid-tight chambers. In this example, the fluid-tight chamber 11a′″″″″, 10e′″″ of the container carrier is formed by the container frame 11a′″″″″ and an interior chamber 10e′″″ of the container carrier 10′″″. In this example, container frame 11a′″″″″ can be made of rigid material or flexible material. Preferably, the interface formed between the container frame 11a′″″″″ and an interior chamber 10e′″″ of the container carrier 10′″″ is gas-tight. In a preferred example, the body of the medicament container is a flexible bag. In a preferred example, the fluid outlet of the medicament container M is configured to be fluidly connected to a tube set 3. Preferably, the medicament container is configured to be attached to the tube set 3. The tube set 3 comprises the delivery tube 31. Preferably, the fluid outlet of the medicament container is configured to be connected to a medicament delivery member via the delivery tube. In this example, the fluid outlet of the medicament container is configured to be attached at one end of the delivery tube; and the medicament delivery member is configured to be attached at the other end of the delivery tube. Preferably, the tube set comprises a piercing member 32 configured to penetrate the fluid outlet of the medicament container M to establish fluid communication between the delivery tube 31 and the medicament container M, as shown in FIG. 34C. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve. Preferably, same sizes of container frames 11a′″″″″ can be provided for different volume of medicament respectively, as shown in FIG. 32.

In the twenty-fifth example, the cassette comprises the container carrier 10′″″ with one fluid-tight chambers. In this example, the fluid-tight chamber 11a″″″″″, 11b″″″″″ of the container carrier is formed by the container frame 11a′″″″″ and a cap 11b″″″″″ configured to be attached to the container frame 11a″″″″″. In this example, container frame 11a′″″″″ can be made of rigid material or flexible material. In a preferred example, the body of the medicament container is a flexible bag. In a preferred example, the fluid outlet of the medicament container M is configured to be fluidly connected to a tube set 3. Preferably, the medicament container is configured to be attached to the tube set 3. The tube set 3 comprises the delivery tube 31. Preferably, the tube set 3 is a part of the cap 11b″″″″″. Preferably, the fluid outlet of the medicament container is configured to be connected to a medicament delivery member via the delivery tube. In this example, the fluid outlet of the medicament container is configured to be attached at one end of the delivery tube; and the medicament delivery member is configured to be attached at the other end of the delivery tube. Preferably, the tube set 3 comprises a piercing member 32 configured to penetrate the fluid outlet of the medicament container M to establish fluid communication between the delivery tube 31 and the medicament container M, as shown in FIG. 34C. Preferably, the piercing member 32 is configured to pierce the fluid outlet M1′″ of the medicament container M once the cap 11b″″″″″ is attached to the container frame 11a′″″″″. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve. In this example, the container carrier partially receives the medicament container and the fluid-tight chamber, as shown in FIG. 33. In a preferred example, different sizes of the container carriers can be provided for different volume of medicament respectively, as shown in FIG. 33.

In the twenty-sixth example, the cassette comprises the container carrier 10′″″ with one fluid-tight chambers. In this example, the fluid-tight chamber 11a″″″″″, 11b″″″″″ of the container carrier is formed by the container frame 11a′″″″″ and a cap 11b″″″″″ configured to be attached to the container frame 11a″″″″″. In this example, container frame 11a′″″″″ can be made of rigid material or flexible material. In a preferred example, the body of the medicament container is a flexible bag. In a preferred example, the fluid outlet of the medicament container M is configured to be fluidly connected to a tube set 3. Preferably, the medicament container is configured to be attached to the tube set 3. The tube set 3 comprises the delivery tube 31. Preferably, the tube set 3 is a part of the cap 11b″″″″″. Preferably, the fluid outlet of the medicament container is configured to be connected to a medicament delivery member via the delivery tube. In this example, the fluid outlet of the medicament container is configured to be attached at one end of the delivery tube; and the medicament delivery member is configured to be attached at the other end of the delivery tube. Preferably, the tube set 3 comprises a piercing member 32 configured to penetrate the fluid outlet of the medicament container M to establish fluid communication between the delivery tube 31 and the medicament container M, as shown in FIG. 34C. Preferably, the piercing member 32 is configured to pierce the fluid outlet M1′″ of the medicament container M when the cap 11b″″″″″ is attached to the container frame 11a′″″″″ and a piercing trigger is activated. In one example, the piercing trigger can be a push button connected to the piercing member 32 such that the piercing member 32 is configured to pierce the fluid outlet M1′″ of the medicament container M when the push button is push towards the medicament container M. Alternatively, the piercing member is connected to a biasing member and being biased towards the medicament container M; a latch is configured to hold the piercing member against the biasing member. In this example, the latch is moved away from the piercing member when the piercing trigger is activated, e.g., in a mechanical way or an electrically way, e.g., the latch can be a Solenoid latch. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve. In this example, the container carrier partially receives the medicament container and the fluid-tight chamber, as shown in FIG. 33. In a preferred example, different sizes of the container carriers can be provided for different volume of medicament respectively, as shown in FIG. 33.

In the twenty-seventh example, the cassette comprises the container carrier with multiple fluid-tight chambers. In this example, each fluid-tight chamber is made of rigid material. In the twenty-seventh example, the container carrier is configured to accommodate multiple medicament containers within the multiple fluid-tight chambers respectively. In this example, each of the multiple medicament containers are identical. Alternatively, each of the multiple medicament containers has the same shape to one another but is sized differently to one another. Alternatively, at least two of the multiple medicament containers are geometrically different from one another. In the twenty-seventh example, each body of each of the multiple medicament container is the flexible bag. Each flexible bag is partially or fully accommodated within one of the multiple fluid-tight chambers so that when the fluid flows into the fluid-tight chamber, the fluid will press on the flexible bag, and thereby expels at least a part of the contained medicament out of the fluid outlet of the medicament container. In this example, the container carrier comprises a connecting port configured to be releasably attached to either the outlet of the fluid pressure power source of the reusable body of the medicament delivery device or another connecting port of another container carrier. In this example, each container carrier is configured to stacked on one another, as shown in FIG. 31. In a preferred example, the body of the medicament container is a flexible bag. In a preferred example, the fluid outlet of the medicament container M is configured to be fluidly connected to a tube set 3. Preferably, the medicament container is configured to be attached to the tube set 3. The tube set 3 comprises the delivery tube 31. Preferably, the fluid outlet of the medicament container is configured to be connected to a medicament delivery member via the delivery tube. In this example, the fluid outlet of the medicament container is configured to be attached at one end of the delivery tube; and the medicament delivery member is configured to be attached at the other end of the delivery tube. Preferably, the tube set comprises the piercing member 32 configured to penetrate the fluid outlet of the medicament container M to establish fluid communication between the delivery tube 31 and the medicament container M, as shown in FIG. 34C. In this example, the medicament container can be completely sealed before use. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve.

In the twenty-eighth example, the cassette comprises the container carrier with multiple fluid-tight chambers. In this example, each fluid-tight chamber is made of rigid material. In the twenty-eighth example, the container carrier is configured to accommodate multiple medicament containers within the multiple fluid-tight chambers respectively. In this example, each of the multiple medicament containers are identical. Alternatively, each of the multiple medicament containers has the same shape to one another but is sized differently to one another. Alternatively, at least two of the multiple medicament containers are geometrically different from one another. In the twenty-eighth example, each body of each of the multiple medicament container is the flexible bag. Each flexible bag is partially or fully accommodated within one of the multiple fluid-tight chambers so that when the fluid flows into the fluid-tight chamber, the fluid will press on the flexible bag, and thereby expels at least a part of the contained medicament out of the fluid outlet of the medicament container. In this example, the fluid-tight chamber of the container carrier is formed by the container frame and an interior chamber of the container carrier. In this example, container frame can be made of rigid material or flexible material. Preferably, the interface formed between the container frame 11a′″″″″ and an interior chamber of the container carrier is gas-tight. In a preferred example, the body of the medicament container is a flexible bag. In a preferred example, the fluid outlet of the medicament container M is configured to be fluidly connected to a tube set 3. Preferably, the medicament container is configured to be attached to the tube set 3. The tube set 3 comprises the delivery tube 31. Preferably, the fluid outlet of the medicament container is configured to be connected to a medicament delivery member via the delivery tube. In this example, the fluid outlet of the medicament container is configured to be attached at one end of the delivery tube; and the medicament delivery member is configured to be attached at the other end of the delivery tube. Preferably, the tube set comprises a piercing member 32 configured to penetrate the fluid outlet of the medicament container M to establish fluid communication between the delivery tube 31 and the medicament container M, as shown in FIG. 34C. Additionally, each fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve. Preferably, same sizes of container frames can be provided for different volume of medicament respectively, as shown in FIG. 32.

In the twenty-ninth example, the cassette comprises the container carrier with multiple fluid-tight chambers. In this example, each fluid-tight chamber is made of rigid material. In the fifteenth example, the container carrier is configured to accommodate multiple medicament containers within the multiple fluid-tight chambers respectively. In this example, each of the multiple medicament containers are identical. Alternatively, each of the multiple medicament containers has the same shape to one another but is sized differently to one another. Alternatively, at least two of the multiple medicament containers are geometrically different from one another. In the fifteenth example, each body of each of the multiple medicament container is the flexible bag. Each flexible bag is partially or fully accommodated within one of the multiple fluid-tight chambers so that when the fluid flows into the fluid-tight chamber, the fluid will press on the flexible bag, and thereby expels at least a part of the contained medicament out of the fluid outlet of the medicament container. In this example, each fluid-tight chamber of the container carrier is formed by the container frame and a cap configured to be attached to the container frame. In this example, container frame can be made of rigid material or flexible material. In a preferred example, the body of the medicament container is a flexible bag. In a preferred example, the fluid outlet of the medicament container M is configured to be fluidly connected to a tube set 3. Preferably, the medicament container is configured to be attached to the tube set 3. The tube set 3 comprises the delivery tube 31. Preferably, the tube set 3 is a part of the cap. Preferably, the fluid outlet of the medicament container is configured to be connected to a medicament delivery member via the delivery tube. In this example, the fluid outlet of the medicament container is configured to be attached at one end of the delivery tube; and the medicament delivery member is configured to be attached at the other end of the delivery tube. Preferably, the tube set 3 comprises a piercing member 32 configured to penetrate the fluid outlet of the medicament container M to establish fluid communication between the delivery tube 31 and the medicament container M, as shown in FIG. 34C. Preferably, the piercing member 32 is configured to pierce the fluid outlet M1′″ of the medicament container M once the cap is attached to the container frame. Additionally, each fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve. In this example, the container carrier partially receives the medicament container and the fluid-tight chamber, as shown in FIG. 33. In a preferred example, different sizes of the container carriers can be provided for different volume of medicament respectively, as shown in FIG. 33.

In the thirtieth example, the cassette comprises the container carrier with multiple fluid-tight chambers. In this example, each fluid-tight chamber is made of rigid material. In the fifteenth example, the container carrier is configured to accommodate multiple medicament containers within the multiple fluid-tight chambers respectively. In this example, each of the multiple medicament containers are identical. Alternatively, each of the multiple medicament containers has the same shape to one another but is sized differently to one another. Alternatively, at least two of the multiple medicament containers are geometrically different from one another. In the fifteenth example, each body of each of the multiple medicament container is the flexible bag. Each flexible bag is partially or fully accommodated within one of the multiple fluid-tight chambers so that when the fluid flows into the fluid-tight chamber, the fluid will press on the flexible bag, and thereby expels at least a part of the contained medicament out of the fluid outlet of the medicament container. In this example, each fluid-tight chamber of the container carrier is formed by the container frame and a cap configured to be attached to the container frame. In this example, container frame can be made of rigid material or flexible material. In a preferred example, the body of the medicament container is a flexible bag. In a preferred example, the fluid outlet of the medicament container M is configured to be fluidly connected to a tube set 3. Preferably, the medicament container is configured to be attached to the tube set 3. The tube set 3 comprises the delivery tube 31. Preferably, the tube set 3 is a part of the cap. Preferably, the fluid outlet of the medicament container is configured to be connected to a medicament delivery member via the delivery tube. In this example, the fluid outlet of the medicament container is configured to be attached at one end of the delivery tube; and the medicament delivery member is configured to be attached at the other end of the delivery tube. Preferably, the tube set 3 comprises a piercing member 32 configured to penetrate the fluid outlet of the medicament container M to establish fluid communication between the delivery tube 31 and the medicament container M, as shown in FIG. 34C. Preferably, the piercing member 32 is configured to pierce the fluid outlet M1′″ of the medicament container M when the cap is attached to the container frame and a piercing trigger is activated. In one example, the piercing trigger can be a push button connected to the piercing member 32 such that the piercing member 32 is configured to pierce the fluid outlet M1′″ of the medicament container M when the push button is push towards the medicament container M. Alternatively, the piercing member is connected to a biasing member and being biased towards the medicament container M; a latch is configured to hold the piercing member against the biasing member. In this example, the latch is moved away from the piercing member when the piercing trigger is activated, e.g., in a mechanical way or an electrically way, e.g., the latch can be a Solenoid latch. Additionally, each fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve.

In this example, the container carrier partially receives the medicament container and the fluid-tight chamber, as shown in FIG. 33. In a preferred example, different sizes of the container carriers can be provided for different volume of medicament respectively, as shown in FIG. 33.

In the thirty-first example, the cassette comprises a cassette housing configured to be releasably attached to the reusable body of the medicament delivery device. The cassette comprises the cassette comprises multiple container carrier with one or more fluid-tight chambers respectively. In this example, each fluid-tight chamber is made of rigid material. In the thirty-first example, the container carrier is configured to accommodate multiple medicament containers within the multiple fluid-tight chambers respectively. In this example, each of the multiple medicament containers are identical. Alternatively, each of the multiple medicament containers has the same shape to one another but is sized differently to one another. Alternatively, at least two of the multiple medicament containers are geometrically different from one another. In the twenty-seventh example, each body of each of the multiple medicament container is the flexible bag. Each flexible bag is partially or fully accommodated within one of the fluid-tight chambers so that when the fluid flows into the fluid-tight chamber, the fluid will press on the flexible bag, and thereby expels at least a part of the contained medicament out of the fluid outlet of the medicament container. In this example, the container carrier comprises a connecting port configured to be releasably attached to either the outlet of the fluid pressure power source of the reusable body of the medicament delivery device or another connecting port of another container carrier. In this example, each container carrier is configured to stacked on one another within the cassette housing, as shown in FIG. 31. In a preferred example, the body of the medicament container is a flexible bag. In a preferred example, the fluid outlet of the medicament container M is configured to be fluidly connected to a tube set 3. Preferably, the medicament container is configured to be attached to the tube set 3. The tube set 3 comprises the delivery tube 31. Preferably, the fluid outlet of the medicament container is configured to be connected to a medicament delivery member via the delivery tube. In this example, the fluid outlet of the medicament container is configured to be attached at one end of the delivery tube; and the medicament delivery member is configured to be attached at the other end of the delivery tube. Preferably, the tube set comprises the piercing member 32 configured to penetrate the fluid outlet of the medicament container M to establish fluid communication between the delivery tube 31 and the medicament container M, as shown in FIG. 34C. In this example, the medicament container can be completely sealed before use. Additionally, the fluid-tight chamber optionally comprises the release valve 115. Additionally, the inlet of the fluid-tight chamber optionally comprises a one-way tube valve, e.g., an umbrella valve or a Belleville spring valve or a ball valve. Alternatively, the inlet of the fluid-tight chamber comprises a multi-directions valve.

It should be noted that the container carrier, as described above examples, can be reusable or disposable. In one example where the container carrier is reusable, the container carrier comprises a main body and a cover sealing the main body. The cover can be completely removed from main body. Alternatively, instead of the cover, the main body comprises a hinge door movably sealing the main body. In this example, the user can return the used cassette to a recycle point or the pharmacy. The used medicament container can be removed from the container carrier. The container carrier now is ready to be used for another new medicament container. In one example where the container carrier is disposable, the container carrier is configured to seal the medicament container once the medicament container is assembled into the container carrier.

It should be noted that when the cassette is configured to be connected to the vacuum device, as mentioned above, the cassette does not need to have the release valve 115 as the vacuum device can expel the fluid within the fluid-tight chamber as the release valve.

Another aspect of the invention provides a medicament delivery device 2; 2; 2″ comprises the cassette as mentioned above. The medicament delivery device comprises a reusable body 20; 20′; 20″; 20′″ and a changeable medicament delivery member 23; 23′a, 23bd, 23c′, 23d′. The reusable body 20; 20′; 20″; 20′″ of the medicament delivery device 2; 2; 2″ comprises the fluid pressure power source 21 connected to the inlet 110; 110′; 110″; 110′″; 110″″; 110a of the fluid-tight chamber 11; 11′; 11″; 11a′″, 11b′″″, 11c′″; 11a″″, 11b″″, 11c″″, 11d″″; 11′″″, 11″″″, 11a′″″, 11b′″″″, 11e′″″″, 11d′″″″, 11a′″″″, 11b′″″″, 11c′″″″, 11d′″″″. In a preferred example, the fluid pressure power source is pneumatic power source so that the pneumatic power source can output gas, e.g., air or nitrogen, into the fluid-tight chamber. Alternatively, the fluid pressure power source is hydraulic power source, so that the hydraulic power source can output liquid, e.g., water or oil, into the fluid-tight chamber.

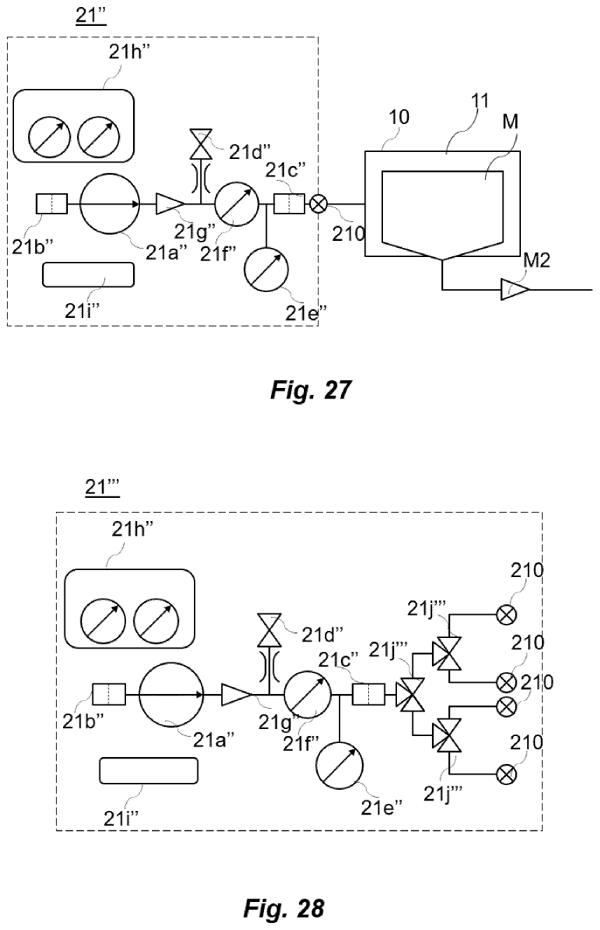

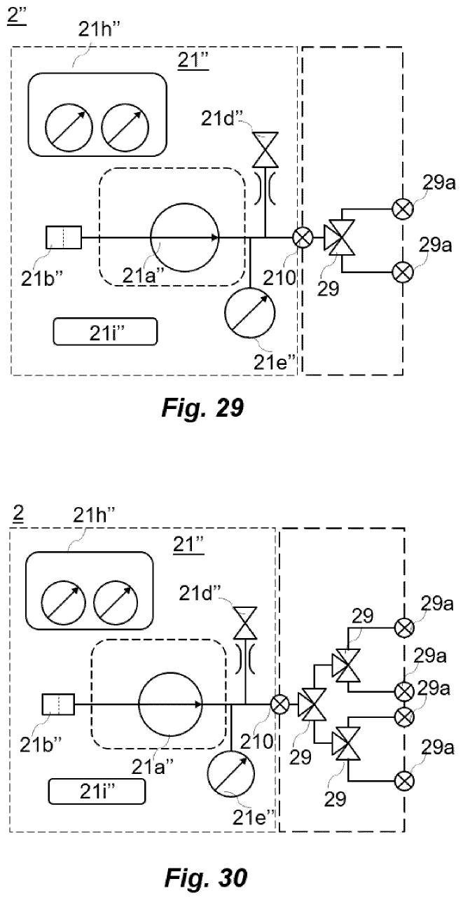

In one example, as shown in FIG. 27, the fluid pressure power source 21″; 21′″ comprises a fluid pump 21a″ having an inlet fluidly connected to the environment and an inlet filter 21b″ connected to the inlet of the fluid pump 21a″ such that the contamination from the environment, e.g., dust, can be prevented from entering to the fluid pump 21a″. Thus, the ingress protection of the fluid pressure power source can be IP56 level. In a preferred example, the fluid pressure power source 21″; 21′″ comprises a pump-outlet check valve 21g″, followed by a downstream controllable release valve 21d″ (venting to atmosphere), a flow sensor 21f′, a pressure sensor 21e″, an outlet filter 21c″configured to be connected to the fluid-tight chamber. The fluid pressure power source 21″; 21′″ optionally connected to a compensation block 21h″ and a controller 21i″. The controller 21i″ can be a processor within the reusable body of the medicament delivery device connected to the fluid pressure power source; or the controller 21i″ can be assembled in the fluid pressure power source. The compensation block 21h″ will be explained in detail later. The flow sensor and the pressure sensor are optional for the fluid pressure power source 21″; 21″. For example, the examples as shown in FIGS. 29-30 do not have flow sensor. It should be noted that, the flow sensor within the fluid pressure power source us configured to measure the fluid flow of the fluid pressure power source.

In another example, as shown in FIG. 28, the fluid pressure power source 21′″ further comprises one or more multi-directions valve 21j′″, e.g., 2/2-way valve, 3/2-way valve, 5/2-way valve. The outlet port of each multi-directions valve defines the outlet 210 of the fluid pressure power source 21′″. In this example, the fluid pressure power source 21′″ can connect to multiple cassettes and/or multiple fluid-tight chamber in one cassette.

Alternatively, or additionally, the medicament delivery device 2″ comprises a multi-directions valve 29, connected to the fluid pressure power source 21″. In this example, the outlet 210 of the fluid pressure power source 21″ is configured to connect to a port of the multi-directions valve 29; and the other ports 29a of the multi-directions valve 28 are configured to be attached to the fluid-tight chamber, as shown in FIGS. 29-30. In this example, the inlet of the fluid-tight chamber is fluidly connected to the outlet 210 of the fluid pressure power source via the multi-directions valve 29. As a result, one fluid pressure power source 21″ can connect to multiple fluid-tight chambers, and thus, connected to multiple medicament containers.

Furthermore, the fluid pump 21a″ can be a piezo pump, a motor-based fluid pump, a piston pump, or a diaphragm pump.

The medicament delivery device comprises the changeable medicament delivery member 23; 23′a, 23bd, 23c′, 23d′ configured to connect to the fluid outlet M1; M1′; Ma1, Mb1, Mc1 of the medicament container M; Ma, Mb, Mc, Md; Ma′, Mb′, Mc′, Md′. In a preferred example, the medicament delivery device 2 is configured to deliver multiple medicaments and/or large volume medicament to the patient.

In one example, the medicament delivery device comprises the tube set 3. As mentioned above, the tube set 3 comprises the delivery tube 31. The fluid outlet M1′″ of the medicament container M is configured to be attached to one end of the delivery tube 31; and the medicament delivery member 23 is configured to be attached to the other end of the delivery tube 31. In this example, the medicament delivery member 23 and the tube set 3 are both changeable. In a preferred example, the tube set 3 comprises the piercing member 32 configured to establish the fluid connection between the delivery tube 3 and the medicament container M as mentioned above.

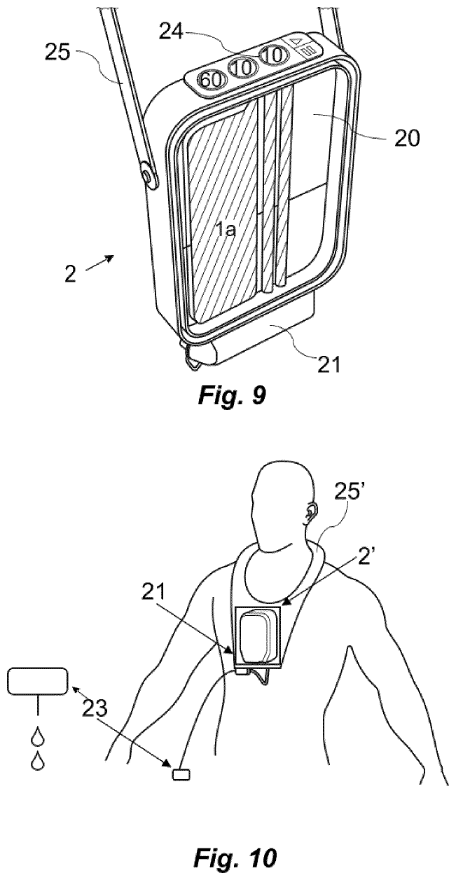

As the medicament delivery device 2; 2; 2″ is configured to deliver multiple medicaments and/or large volume of medicament to the patient, the medicament delivery time could be long, e.g., couple of hours. Thus, in a preferred example, the medicament delivery device is portable. Therefore, the user can easily carry the medicament delivery device 2 for moving, and travelling, also, the patient can easily carry the medicament delivery device 2 during the medicament delivery operation. In a preferred example, the medicament delivery device comprises a user wearable feature 25; 25′; 25″ connected to the reusable body. The user wearable feature is preferably not configured to be adhesively attached to a patient's skin, e.g., not attached to a user via an adhesive material as a skin contact with an adhesive material is easily to cause a skin reaction, e.g., infections, heat, allergens, and/or a rash. In the preferred example, the user wearable feature is a belt, shoulder straps, neck strap, a vest, a harness, a belt clip, portions thereof or a combination of thereof, as shown in FIGS. 9-10, 17-19 and 20-21.

Therefore, the user can easily carry the medicament delivery device 2; 2; 2″. Furthermore, the patient can easily carry the medicament delivery device 2; 2; 2″ during the medicament delivery operation. Therefore, the mobility of the patient will have fewer limitations during the medicament delivery operation. In a preferred example, the user wearable feature be provided with one or more of a persistently antimicrobial, antifungal, or antiviral agent. In a preferred example, the user wearable feature is provided with a coating comprising fibers woven into the fabric material (e.g., silver fibers), be provided through a secondary coating, spray, or dipping operation, or by selecting an outer fabric layer featuring persistently antimicrobial, antifungal, or antiviral properties.

In a preferred example, the medicament delivery device is an injection device, e.g., an infusion device or an on-body injector. In this example, the medicament delivery member is an injection needle or an insertion needle with a soft cannula.

The fluid pressure power source 21 is configured to generate/release pressurized fluid, e.g., liquid or gas, and deliver the pressurized fluid into the fluid-tight chamber. In a preferred example, the fluid pressure power source 21 is configured to generate/release pressurized gas. In one example, the fluid pressure power source can be a pressurized gas canister. In a preferred example, the fluid pressure power source comprises a piezo pump. Alternatively, the fluid pressure power source comprises a motor-based fluid pump, e.g., a diaphragm pump or a piston pump, as mentioned above.

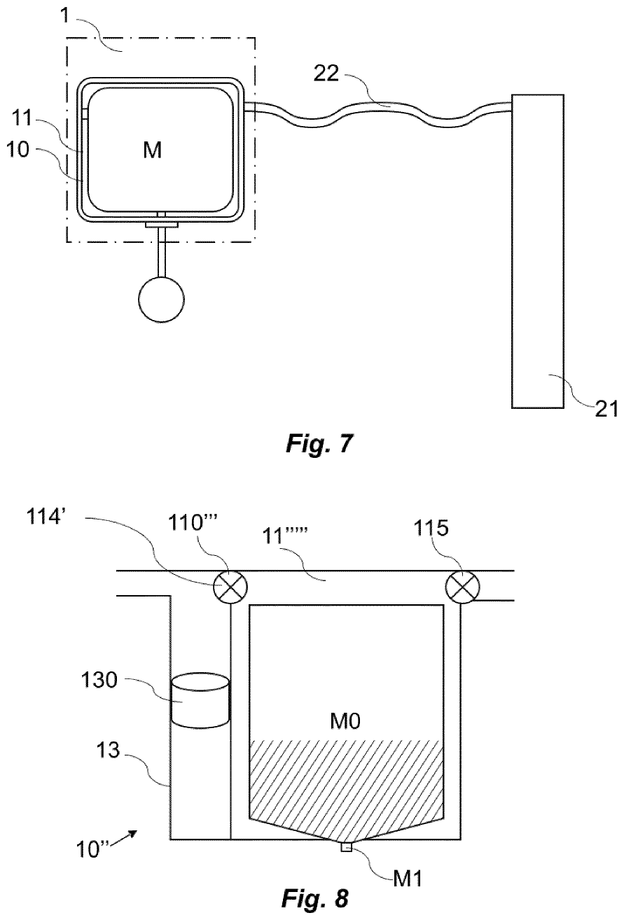

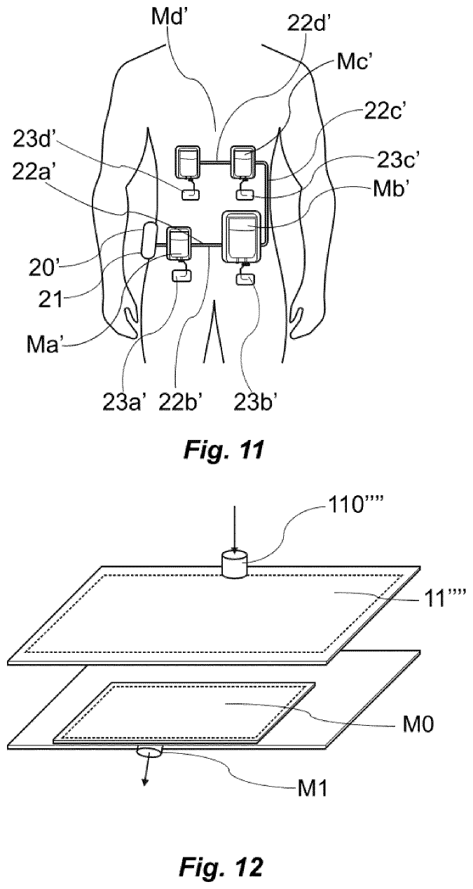

In one example, the fluid-tight chamber is adjacent to the fluid pressure power source. Therefore, the fluid pressure power source is fluidly connected to the inlet of the fluid-tight chamber directly. Alternatively, a transmission tube 22 is arranged between the fluid pressure power source 21 and the inlet of the fluid-tight chamber 11, as shown in FIG. 7. In this example, the fluid pressure power source is fluidly connected to the inlet of the fluid-tight chamber via the transmission tube 22. In one example, the medicament delivery device is configured to be attached to at least two medicament containers Ma, Mb, Mc, Md, as shown in FIG. 11. In this example, the fluid pressure power source 21 is connected to one fluid-tight chamber via the transmission tube 22a′, and indirectly connected to other fluid-tight chambers via other transmission tubes 22b′, 22c′, 22d′. The transmission tubes 22a′, 22b′, 22c′, 22d′ are configured to direct the output fluid from the fluid pressure power source to each individual fluid-tight chamber. In one example where the fluid pressure power source of the medicament delivery device is connected to more than one cassette, the connection as disclosed in the embodiment as shown in FIG. 11 is also applicable. In the example as shown in FIG. 11, the multiple cassettes are configured to be connected to the fluid pressure power source 21 via multiple transmission tubes 22a′, 22b′, 22c′, 22d′. In this example, the reusable body 20′ is configured to accommodate the fluid pressure power source 21 and optionally accommodate one or more electronic components, e.g., piezo pump, a processor(s), wireless communication unit(s), touch screen(s), display(s), microphone(s), LED light(s), speaker(s), vibration motor(s), accelerometer(s), gyro sensor(s), memory, temperature sensor(s), temperature conditioning unit(s) . . . , etc., a battery. In one example, the reusable body 20 In a preferred example, the reusable body 20′ is compact and easy to be carried.

In one example, each transmission tube extends between two opposite ends. Each transmission tube comprises two screw connecting heads attached at the two opposition ends of the transmission tube respectively. In this example, the fluid pressure power source 21′ of the medicament delivery device comprises a counter screw head, the user can screw one transmission tube 22a′ to a cassette and to the fluid pressure power source 21′ of the medicament delivery device so that the fluid pressure power source 21′ of the medicament delivery device is fluidly connected to the transmission tube. The user can screw the rest of transmission tubes between two cassettes. In this example, all transmission tubes can be identical in this example so that the manufacture cost can be reduced, and it is also easy to be used for the user. In one example where the cassette comprises the cassette housing, two counter screw heads is arranged in a wall of the cassette housing. Alternatively, two counter screw heads are arranged in walls of the fluid-tight chamber. Instead of screw connection, a Luer connection or a bayonet connection can be used between the transmission tube, the cassette, and the fluid pressure power source. Therefore, the cassette can be releasably attached to the medicament delivery device.

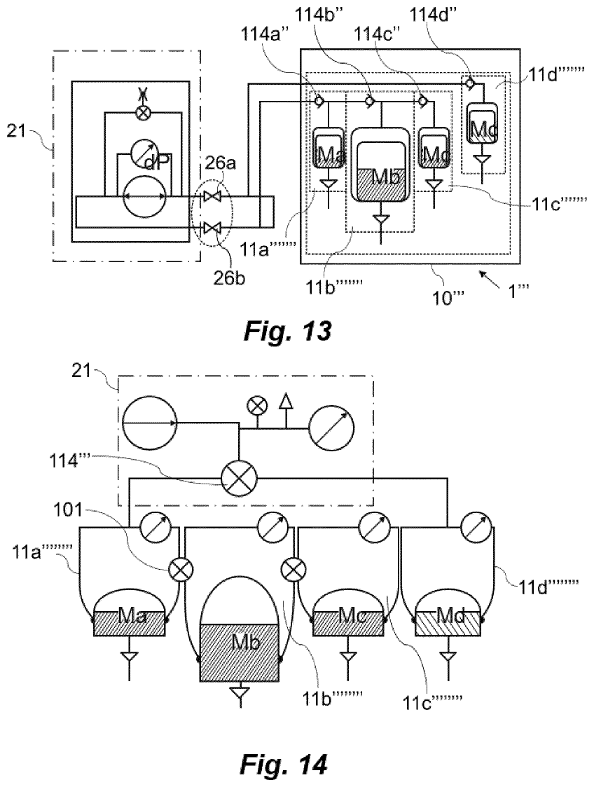

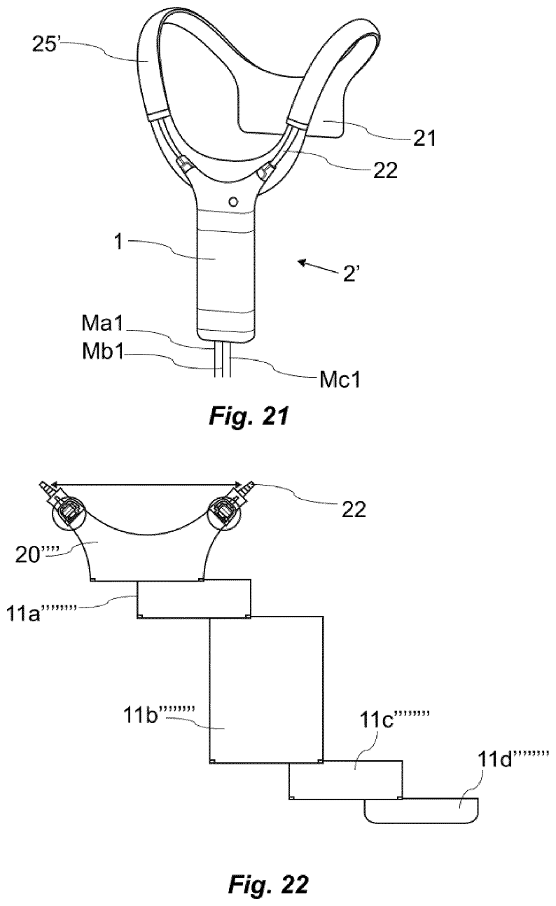

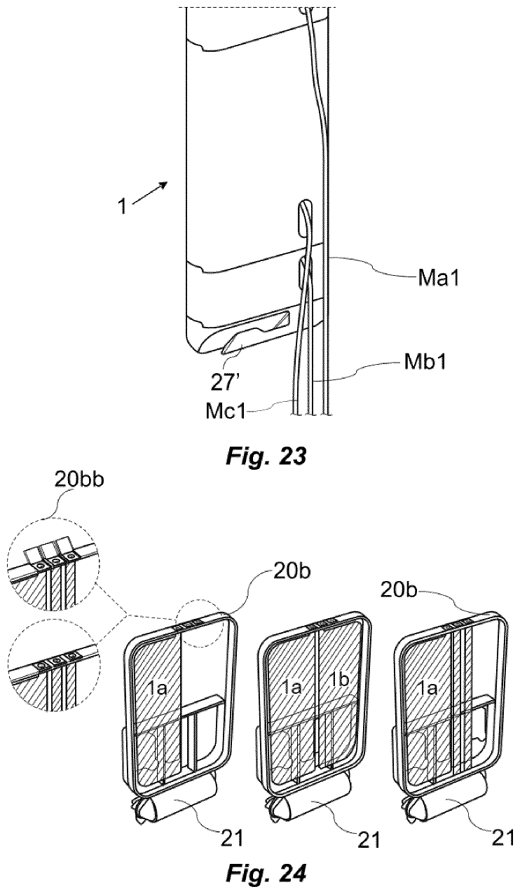

Alternatively, as shown in FIGS. 21-23, the reusable body 20″″ comprises a base 20″″. The base 20″″ is configured to accommodate the fluid pressure power source 21. In a preferred example as shown in FIGS. 21-23, the user wearable feature 25′ of the reusable body 20″″ comprises a neck strap. In this example, the base 20″″ comprises a front part configured to be placed next to a patient's chest and a rear part configured to be placed next to the patient's back. In this example, the cassette comprises multiple fluid-tight chambers 11a′″″″, 11b′″″″, 11c′″″″, 11d′″″″, as shown in FIGS. 13, 14 and 22. As mentioned above, each fluid-tight chamber comprises one of the multiple medicament container Ma, Mb, Mc, Md, as shown in FIGS. 21 and 23. In this example, the cassette is configured to be releasably attached to the base 20″″, e.g., by snap-fit or a groove-and-ridge connection. In a preferred example, at least two of the multiple medicament containers comprises a fluid outlet Ma1, Mb1, Mc1 that is fluidly separated from a fluid outlet of any other one of the multiple medicament containers. Therefore, at least two medicament containers are provided with a fluidically separate connection to the patient in a manner by which separate medications do not mix during administration unless desired at the patient site. It should be noted that, the connection between fluid-tight chambers as shown in FIG. 22 is can also be used as a connection to connect multiple different cassette or multiple different container carriers. Furthermore, as shown in FIG. 21, the transmission tube 22 extends from the rear part of the base 20″″ to the front part of the base 20″″ so that the output fluid from the fluid pressure power source 21 can be transmitted to the fluid-tight chamber of the cassette.

An over the shoulder design, namely, the design as shown in FIG. 10 and FIGS. 20-23, distributes weight of the device across the patients back, chest, and shoulders, as seen in FIG. 10. Worn over or under clothing, it provides a short path to the abdomen site. As seen in FIG. 20, the fluid pressure power source 21 is placed on the rear part of the base and is a reusable component. The fluid pressure power source 21 accommodates both pneumatic and mechanical drive systems for flexibility. In one preferred embodiment, the fluid pressure power source 21 also has a battery that may be recharged in between uses of the device. In an alternative embodiment, the fluid pressure power source 21 does not have the battery, and the battery is provided as a detachable component (i.e. module as discussed below) on the front or back. Shoulder straps are designed for comfort, even when reusable body contains the maximum medication volume. These are used to conceal one or more tubes and/or wires making electrical, optical, pneumatic, or other connections from the front part to the rear part. As seen in FIG. 21, the cassette(s) is situated on the front part of the base and is disposable comments that may be arranged in accordance with a desired medication sequence or regimen. The cassette may be prefilled by pharmacy, a pharmaceutical manufacturer, or both. The cassette may be attached in sequence, or alternatively, the cassette may be connected by a patient one at time if low weight is desired, but this is not expected to be the preferred commercial embodiment.

As seen in FIG. 22, large and small volume concepts may also stack in a variety of configurations for more complex regimens and automated delivery. The cassette contains a flexible bag (in a preferred embodiment; but syringes, cartridges, or other container are possible) with outer rigid cassettes sized for the volumes they contain. When multiple cassettes are used, they may be provided with their own individual in process or end of dose feedback indicators, e.g., lights/LEDs, or feedback may be provided in the upper unit with the wired connections (or both the upper unit and cassettes).

Cassettes are filled by the pharmacist or selected from pre-filled options, and then securely assembled in sequence (slide, click, etc.) and capped to complete the preparation steps. Other cassettes may also be interspersed. This forms a single unit that is shipped to a patient using common cold-chain shipping, as is used for autoinjectors. The patient receives the assembled cartridges and, with the couplers, attaches them to the drive unit umbilical(s), as shown in FIG. 21. Although FIG. 21 shows two lines, as many lines as desired may be connected, or alternatively, each attachment point may make more than one connection, as with a multiple lumen or multiple conductor sets. One line can be optionally dedicated to emergency drug delivery if required by the patient's regimen. The barbed fitting of FIG. 22, and the removable connection assembly of barbed tubing and release mechanism of FIG. 21 may be replaced by any suitable connector, ideally designed for secure attachment, easy intentional removal, and difficult unintentional removal, and universal design principles to accommodate a wide variety of user populations. FIG. 21 also illustrates an optional power button and also the status lights on each module illuminated. As shown in FIG. 23, the end cap which completes the sequence also incorporates a tie-bar style clip 25″ to secure the reusable body for active users. This clip 25″ may be placed in the center seam of a shirt or may be provided with a magnetic closure on the other side of the shirt if a seam is not present. FIG. 21, or other similar features, optionally may be included to prevent undesired dangling of the reusable body during infusion, especially as a user wearing the device leans forward. Here in FIG. 23, the side view of the modules is also visible, showing the interlocking features. FIG. 23 also shows optional tubing strain reliefs; other tubing management features may be available based on the length and diameter of tubing, number of tubes, and intended injection site(s).

Alternatively, FIGS. 21-23 can also schematically show the medicament delivery device comprises a reusable body with multiple sections for receiving multiple cassettes. In this example, each of the multiple fluid-tight chambers 11a″″″″, 11b″″″″, 11c″″″″, 11d″″″″ in FIG. 22 are received within different sections of the reusable body respectively.

Furthermore, alternatively, instead of a cascade connection between the reusable body 20″″ of the medicament delivery device 2′ and the one or more cassette, as shown in FIGS. 21-23, the reusable body 20; 20′; 20″; 20′″ of the medicament delivery device 2 is configured to accommodate one or more cassettes, as shown in FIGS. 9-10 and 17-19. In this example, the reusable body comprises an inner section for accommodating the one or more cassettes. The cassette can be attached to the inner section of the reusable body via magnetic connection, a releasably snap-fit connection, a screw thread connection, and/or a bayonet connection. Therefore, the cassette can be releasably attached to the medicament delivery device.

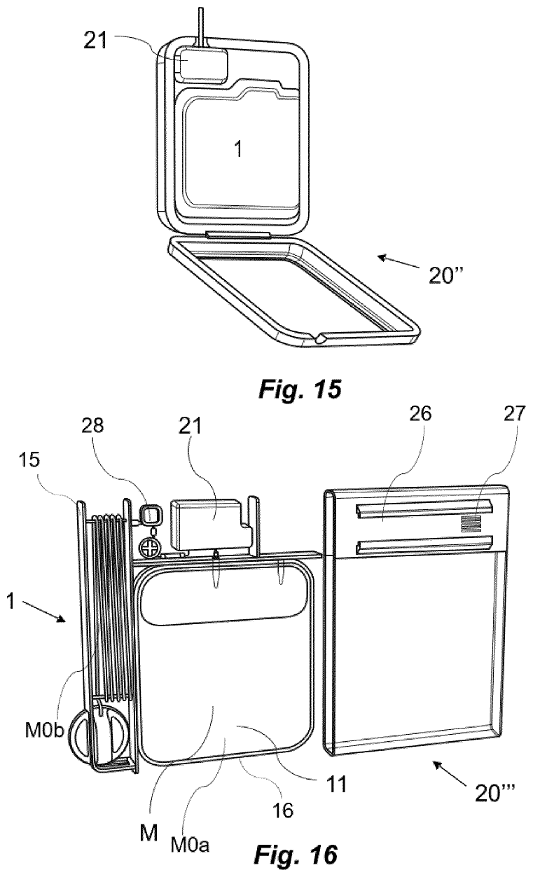

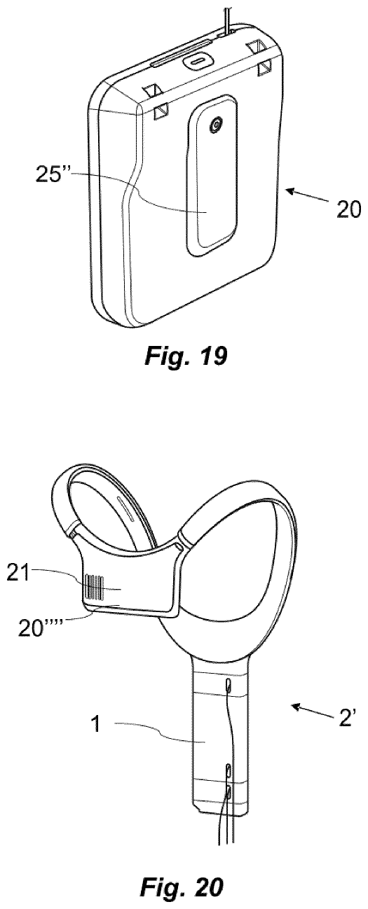

In one example, the fluid pressure power source 21 is attached to the inner section of the reusable body 20″, as shown in FIGS. 15 and 17. In another example, the fluid pressure power source 21 is attached to a cassette and the cassette is attached to the inner section of the reusable body 20″, as shown in FIGS. 16 and 18. In one example, the reusable body 20″ comprises two covers connected together via a hinge, as shown in FIGS. 15 and 17. Alternatively, the reusable body 20′″ comprises a wall around the inner section, in other words, the inner section of the reusable body 20′″ is the space with a boundary that is provided by the wall, as shown in FIGS. 16 and 18. In this example, the wall around an opening where the cassette is configured to be placed into the inner section through the opening, e.g., the user inserts the cassette into the inner section of the reusable body 20′″ via through the opening. In this example, the cassette can be attached to the inner section of the reusable body 20″ via a groove-and-ridge connection.

It should be noted that in the example as shown in FIG. 16 and FIG. 18, instead of being attached to the cassette, the fluid pressure power source can be releasably attached to the inner section of the reusable body independently. For example, the fluid pressure power source can be inserted into the inner section of the reusable body followed by the cassette being inserted into the inner section. In this example, the fluid pressure power source can be reusable or disposable. Furthermore, in one example where the medicament container is a combination of the flexible bag M0a and the delivery tube M0b extending from the flexible bag, as shown in FIG. 16, the flexible bag MO of the medicament container M is placed within the fluid-tight chamber 11 of the cassette. The delivery tube M0b is wound around a frame 15 of the cassette. In this example, the cassette is configured to be partially placed within the inner section of the reusable body 20′″, as shown in FIG. 18. When the cassette is placed into the reusable, the frame 15 and the delivery tube M0b is positioned outside of the inner section of the reusable body 20′″. Additionally, in another example, the cassette provides a filter 28 connected to the delivery tube M0b. In a preferred example, the filter 28 is configured to expel air bubbles that are contained in the contained medicament such that the air bubbles will not be delivered into the user's body.

Furthermore, instead of having the fluid-tight chamber 11 as a part of the cassette as mentioned above, the fluid-tight chamber 11 can be found by the cassette and the reusable body. For example, the cassette comprises a medicament container frame 16, as shown in FIG. 16, configured to surround the medicament container M. In this example, the medicament container M is attached to the medicament container frame 16 and is configured to be inserted into the inner section of the reusable body. When the medicament container frame 16 is placed into the inner section of the reusable body, the inner section of the reusable body together with the medicament container frame 16 form the fluid-tight chamber. In this example, the cassette can be made of less plastic material, thus, the cost of the cassette can be reduced. In another example, the medicament container frame 16 comprises a connector to the fluid pressure power source 21, for example, a valve; therefore, the fluid pressure power source can only be activated to release the fluid into the fluid-tight chamber once the medicament container frame 16 is inserted into the inner section of the reusable body. Additionally, the reusable body 20′″ comprises a connection track 26. In one example as shown in FIG. 16, the connection track 26 is formed by two ribs. The connection track 26 enables multiple reusable bodies 20′″ to be attached together; for example, the reusable body comprises the connection track 26 at one side and a connection protrusion at the other side opposite to the connection track 26. The connection protrusion is configured to be attached to the connection track 26 by being slid along the connection track 26. In this example, the reusable body comprises a communication spot 27 configured to connect to a counter communication unit of another reusable body. For example, the communication spot 27 is arranged within the connection track 26, as shown in FIG. 16; and the counter communication unit is arranged in the connection protrusion. The communication spot can be a conductive spot configured to be in contact with and electrically connected to the counter communication unit. Alternatively, the communication spot 27 can be an RFID/NFC circuit that is configured to be connected with the counter communication unit via a contact free connection. In this example, the user interface 24 comprises the counter communication unit configured to be connected to the communication spot 27. In a preferred example, the user interface 24 comprises the counter communication unit so that the user interface 24 can be attached to the reusable body by being attaching to the connection track 26. An example as shown in FIG. 26, two reusable bodies 20′″ with two cassettes inserted respectively are attached to one another. The user interface 24 is attached to one of the two reusable body 20′″. In this example, the user interface 24 is configured to detect how many cassettes are connected, via the connection between the communication spot and the counter communication unit, followed by controlling the medicament in different cassette to be delivered.

In a preferred example, the user wearable feature 25″ is a belt clip, as shown in FIG. 19, in the example where the reusable body is configured to accommodate the cassette.

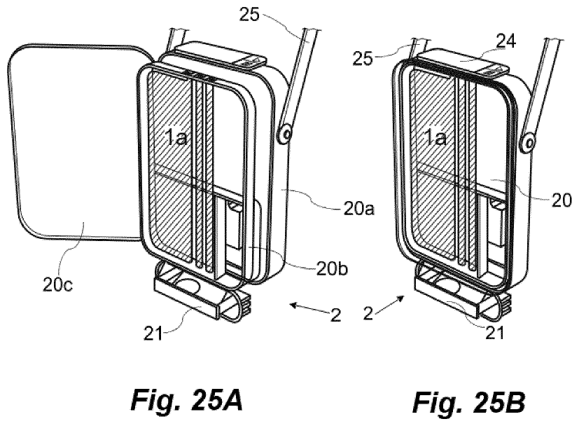

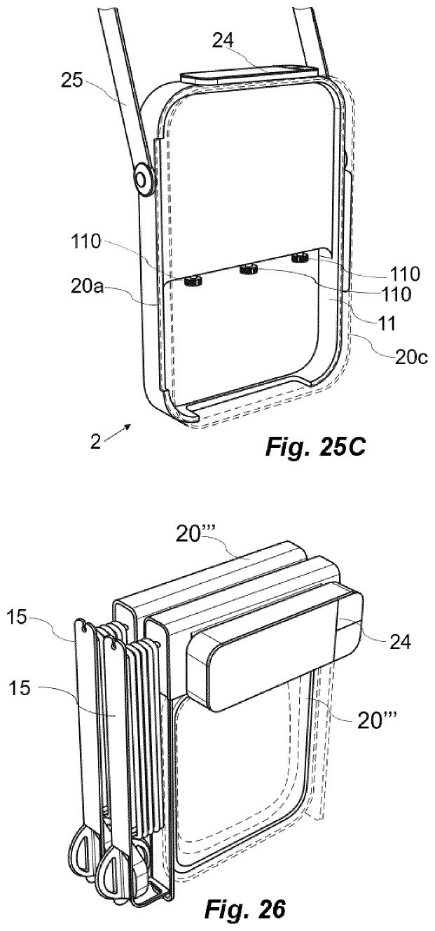

In another example, as shown in FIGS. 9, 24-25, and 31, the reusable body 20 of the medicament delivery device 2 is configured to accommodate multiple more cassettes 1a, 1b. In this example, the reusable body 20 comprises an inner section for accommodating the more cassettes 1a, 1b. In this example, the reusable body 20 comprises a main body 20a comprising the inner section, optionally a frame 20b configured to be attached with more cassettes 1a, 1b, and a lid 20c configured to seal the main body 20a. Similar to the previous example, the cassettes 1a, 1b can be attached to the inner section of the reusable body via magnetic connection, a releasably snap-fit connection, a screw thread connection, and/or a bayonet connection. In a preferred example, the cassettes 1a, 1b can be attached to the frame 20b of the reusable body 20 via magnetic connection, a releasably snap-fit connection, a screw thread connection, and/or a bayonet connection. Therefore, when the frame is releasably attached to the main body 20a, the cassette can be releasably attached to the medicament delivery device. In this example, the reusable body 20 contains one or more medicament container (flexible bags) and filling ports for use in pharmacy cleanrooms or pharmaceutical filling suite. In one example, the frame 20b comprises one or more tube openings 20bb for being connected to one or more tubes and/or medicament delivery members respectively. In a preferred example, the frame 20b has an integral tube sets and delivery members, e.g., needles. Tube sets and needles are gathered into a tubing management sleeve (bottom of the reusable body) that allows for compact shipment and avoids tangles prior to use and during needle application. Alternatively, the tubing may be optionally terminated in a modular connector, and the tubing set and needle(s) attached separately as part of the use process. The reusable body contains a battery, the fluid pressure power source, and a medicament container interface, e.g., via the lid 20c. The design is intentionally insensitive to orientation or gravity. The form factor has a simple set of use steps that may be performed multiple times. In this example, the user inserts the cassette into the medicament delivery device as seen in FIGS. 25A-B, attaches the needles to the injection sites and simply presses the start button to administer the full treatment regimen. For multiple medication regimens, the device may remind the user when to proceed, or reject medications loaded out of sequence. In one example, the fluid-tight chamber 11 is formed by the reusable body 20 and the frame 20b. In this example, the inlet 110 of the fluid-tight chamber 11 is positioned within the reusable body 20, as shown in FIG. 25C. Additionally, multiple inlets 110 of the fluid-tight chamber 11 can be provided, as shown in FIG. 25C.

Alternatively, FIGS. 9, 24-25, 32 can also schematically show the medicament delivery device comprises one cassette with multiple medicament containers. In this example, the body 20 and the frame 20b that are both described as a part of the reusable body of the medicament delivery device should be considered as a part of the cassette in this example. In other word, in this example, the reusable body of the medicament delivery device only comprises the fluid pressure power source 21 and additionally the user interface 24 and the user wearable feature 25. In this example, the body (with the reference sign 20 in FIGS. 24-25B) and the frame (with the reference sign 20b in FIGS. 24-25B) are the container carrier of the cassette. In this example, the container carrier of the cassette is reusable. As mentioned above, the frame is configured to be attached with more medicament containers (with the reference sign 1a in FIGS. 24-25B), and a lid (with the reference sign 20c in FIGS. 24-25B) is configured to seal the main body with the reference sign 20a in FIGS. 24-25B) of the cassette. Similar to the previous example, the medicament containers can be attached to the inner section of the reusable body via magnetic connection, a releasably snap-fit connection, a screw thread connection, and/or a bayonet connection. In a preferred example, the medicament containers can be attached to the frame of the reusable body via magnetic connection, a releasably snap-fit connection, a screw thread connection, and/or a bayonet connection. Therefore, when the frame is releasably attached to the main body, the medicament container can be releasably attached to the cassette. Similarly, in this example, the frame comprises one or more tube openings (with the reference sign 20bb in FIGS. 24-25B) for being connected to one or more tubes and/or medicament delivery members respectively. In a preferred example, the frame has an integral tubing sets and needles. Tubing sets and needles are gathered into a tubing management sleeve (bottom of the reusable body) that allows for compact shipment and avoids tangles prior to use and during needle application. Alternatively, the tubing may be optionally terminated in a modular connector, and the tubing set and needle(s) attached separately as part of the use process. In this example, the main body and the lid formed the fluid-tight chamber, therefore, when the fluid flows into the main body, the fluid can press the medicament contained within the medicament container out and delivery via the medicament delivery member to the patient. In this example, the patient connects the cassette to the fluid pressure power source 21 as seen in FIGS. 25A-B, attaches the needles to the injection sites and simply presses the start button to administer the full treatment regimen.

In another example, the medicament delivery device 2; 2′; 2″ comprises a processor electrically connected to the fluid pressure power source 21, an electrical power source, e.g., battery, connected to the processor. The processor and the electrical power source are accommodated within the reusable body 20; 20′; 20″; 20′″. In this example, the controller 21i″ as mentioned above, can be the processor of the medicament delivery device or is electrically connected to the processor.

In another example, the medicament delivery device 2; 2′; 2″ comprises a user interface 24, as shown in FIGS. 9 and 18, attached to the reusable body 20; 20′; 20″; 20′″. The user interface 24 is electrically connected to the processor. In one example, the user interface is a button protruding from an outer surface of the reusable body. In another example, the user interface is a touch panel arranged on an outer surface of the reusable body. Furthermore, in another example, the medicament delivery device comprises an orientation sensor, e.g., a gyro sensor, such that the display, the scree, or the touch panel can always be presented in the right-reading graphic indicia to a user, as shown in FIGS. 37A-37B.

In another example, the medicament delivery device comprises a wireless communication receiver and/or transmitter connected to the processor. The wireless communication receiver and/or transmitter can be any suitable long-distance or short-distance wireless communication technology, e.g., RF, Bluetooth, Zigbee, 3G, 4G, 5G. The wireless communication receiver is configured to receive a wireless signal from a remote device and/or an information tag to the processor. The wireless communication transmitter is configured to transmit a wireless signal from the processor to a remote device or write information in an information tag. In this example, instead of the user interface, the user can use the remote device, e.g., a smartphone, to control and/or monitor the medicament delivery operation. Furthermore, in another example, the communication transmitter is configured to write the used information in the information tag of the cassette. The information can be the residual medicament within the cassette.

In another example, the pressure sensor and/or the position sensor of the piston of the fluid-tight measure chamber and/or the position sensor of the container carrier and/or the flow sensor and/or the sensor evaluating the mass flow into the fluid-tight chamber(s) are electrically connected to the processor. Optionally, a temperature sensor is electrically connected to the processor. In this example, the temperature sensor is configured to monitor the temperature of the fluid-tight chamber. In this example, the processor is configured to control the fluid pressure power source to output fluid into the fluid-tight chamber according to a signal from the pressure sensor and/or the position sensor of the piston of the fluid-tight measure chamber and/or the position sensor of the container carrier. In one example, when the fluid pressure power source is a pneumatic power source, the processor is configured to control the pneumatic power source 21 to output fluid at a constant pressure level that is greater than the resistant pressure value of the target tissue until the medicament container is empty, or a predetermined amount of the contained medicament has been delivered. As the volume of the fluid-tight chamber is known, the residual medicament within the medicament container M; M′ can be calculated by monitoring the pressure level within the fluid-tight chamber based on the application of the Ideal Gas Law. Similarly, in another example, the processor is configured to control that the contained medicament is delivered at a constant delivery rate of the medicament contained within the medicament container by maintaining the pressure level within the fluid-tight chamber to be constant during the medicament delivery operation. In a preferred example, there is no other flow rate control arrangement is used. Therefore, the flow rate is substantially equal to the delivery rate of the medicament contained within the medicament container. In this example, the processor controls the fluid pressure power source to deliver more fluid when the detected pressure level is drop and stops the fluid pressure power source or starts the vacuum device when the detected pressure level is increased.

Additionally, in one example where the body of the medicament container comprises the flexible bag and the flexible tube, instead of using the pressure level to control the delivery rate of the medicament contained within the medicament container as mentioned above, the delivery rate of the medicament contained within the medicament container of the medicament delivery can be control by compressing the flexible tube. For example, the flexible tube can be attached with another fluid-tight tube that is connected to the fluid pressure power source. In this example, when the fluid from the fluid pressure power source flows into the fluid-tight tube, the fluid-tight tube can press on the flexible tube of the medicament container and thus control the delivery rate of the medicament contained within the medicament container of the medicament delivery. Alternatively, as mentioned above, the delivery rate of the medicament contained within the medicament container can be adjusted by manipulating the flexible tube, e.g., by a pinch valve.

In one example where the cassette comprises the medicament container, the medicament container comprises a temperature sensor and/or a timer. The temperature sensor is configured to monitor the temperature of the contained medicament, and the timer is configured to record the storage period of the container medicament. In this example, when the cassette is attached to the reusable body, the processor is configured to be electrically connected to the temperature sensor and/or the timer such that the condition of the contained medicament can be verified by the processor. For example, if the contained medicament was stored for too long or has been exposed to a high temperature that might impact the efficiency of the contained medicament, the processor can generate a warning indication to the user and/or send out a warning signal to a remoter device.

In another example, the fluid pressure power source 21 is connected to at least two fluid-tight chambers 11a′″″, 11b′″″, 11c′″; 11a′″″″, 11b′″″″, 11c′″″″, 11d′″″″; 11a″″″″, 11b″″″″, 11c″″″″, 11d″″″″ as shown in FIGS. 5, 13 and 14. For example, the fluid pressure power source 21 is connected to at least two fluid-tight chambers 11a′″, 11b′″, 11c′″; 11a′″″″, 11b′″″″, 11c′″″″, 11d′″″″ via at least two separate fluid paths, e.g., two independent fluid transmission tubes or a dual-lumen tube, as shown in FIGS. 5 and 13. Alternatively, the fluid pressure power source 21 is connected to at least two fluid-tight chambers 11a″″″″, 11b″″″″, 11c″″″″, 11d″″″″ via one main fluid path with two split sub-fluid path and a control valve, as shown in FIG. 14. In this example, the processor is configured to control the fluid pressure power source to selectively output fluid into at least one the fluid-tight chambers 11a′″, 11b′″, 11c′″; 11a′″″″, 11b′″″″, 11c′″″″, 11d′″″″; 11a″″″″, 11b″″″″, 11c″″″″, 11d″″″″.

In one example where the pressure sensor and/or the position sensor or the fluid-tight measure chamber and/or the position sensor of the container carrier is electrically connected to the processor, the processor is configured to control the fluid pressure power source to selectively output fluid into at least one the fluid-tight chambers according to a signal from the pressure sensor and/or the position sensor or the fluid-tight measure chamber and/or the position sensor of the container carrier and/or a sensor evaluating the mass flow into the fluid-tight chambers.

Furthermore, in another example, the processor is configured to control the fluid pressure power source to output a certain amount of fluid. In this example, the certain amount is predetermined or is dependent on a signal from the pressure sensor and/or the position sensor or the fluid-tight measure chamber and/or the position sensor of the container carrier; thereby only a certain amount of medicament contained within the medicament container can be pressed out of the fluid outlet.

In examples where the medicament delivery device 2 comprises the wireless communication receiver connected to the processor and the medicament delivery device is configured to be attached with the cassette comprising multiple medicament containers, the wireless communication receiver is an RFID reader that is configured to read an RFID tag on the cassette.

In this example, the RFID tag carries information about the flow rate for each individual medicament, delivery sequence among different medicaments, dosage, emergency stop information, etc.

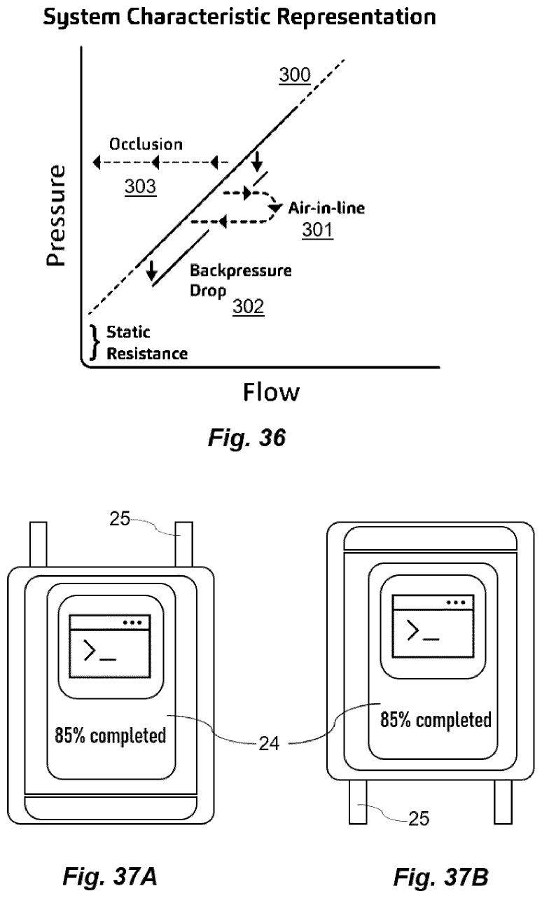

In one example where the fluid-tight chamber comprises a pressure sensor and a flow rate sensor. In a preferred example, the medicament container is a flexible bag fully received within the fluid-tight chamber. In this example, when the user attaches the cassette 1 to the reusable body 20 of the medicament delivery device, the fluid-tight chamber is connected to the fluid pressure power source 21. Before the delivery tube is fluidly connected to the medicament container, the processor can control the fluid pressure power source pressurized the fluid-tight chamber and thus measure the initial condition of the medicament container, e.g., the actual filled volume of the medicament, by the Ideal Gas Law and/or its simplifications as mentioned above. During medicament delivery operation, the processor is configured to continuously monitor the pressure and the flow rate of the fluid-tight chamber via the pressure sensor and the flow rate sensor. As a result, as shown in FIG. 36, comparing to the relation between the flow rate and the pressure in the normal delivery operation 300, if the medicament delivery member is prematurely removed 302, and/or the delivery tube containing air 301 (flow rate increased but the pressure is not increased), and/or delivery occlusion occurs 303 (pressure increased, but the flow rate is not increased), the event(s) can be detected by monitoring the pressure level and the flow rate, thus, the processor can control the fluid pressure power source 21 to stop, slow down; the processor can also control the release valve to open; and the processor can provide feedback to the user. In a preferred example, the one or more processors of the medicament delivery device can be programmed with machine learning module; thus, the judgement of relation between the flow rate and the pressure in the normal delivery operation 300 and/or the changes 301, 302, 303 made by the one or more processor can be self-adjusted during use to increase the accuracy of the event detection.

In one example, as shown in FIG. 13, the fluid pressure power source 21 is connected to the cassette 1 via a dual-lumen tube. In this example, one inner tube of the dual-lumen tube comprises a first valve 26a, and the other inner tube of the dual-lumen tube comprises a second valve 26b. In this example, the fluid pressure power source 21 is connected to a first set of the medicament containers Ma, Mb, Mc via a first valve 26a. In this example, each medicament container is accommodated within one independent fluid-tight chamber 11a′″″″, 11b′″″″, 11c′″″″, 11d′″″″. Each fluid-tight chamber comprises an inlet, and a valve 114a″, 114b″, 114c″ at the inlet. The fluid pressure power source 21 further is connected to a second medicament container Md via a second valve 26b. The second medicament container Md is accommodated within another fluid-tight chamber 11d′″″″ with an inlet and a valve 114d″ at the inlet. In this example, the patient should receive the medicament sequentially from each medicament container Ma, Mb, Mc, and the patient should receive the medicament from the second medicament container M0d only when in an emergency, e.g., an adverse drug reaction appears. In this example, the processor will control the first valve 26a to open and close the second valve 26b. The valve 114a″, 114b″, 114c″ for each fluid-tight chamber can be designed to either with different resistance so that will only open when the previous medicament container is empty or is controlled by the processor. If there is an emergency (either detected by other sensors or the user activates the emergency button as mentioned above), the processor can close the first valve 26a and open the second valve 26d so that the output fluid from the fluid pressure power source can flow into the fluid-tight chamber with the second medicament container Md.

In another example, as shown in FIG. 14, instead of the first and the second valves, the first set of the medicament container Ma, Mb, Mc and the second medicament container Md (the emergency medicament), can be connected to the fluid pressure power source 21 with one multi-direction valve 114′″. In this example, the processor controls the multi-direction valve 114′″ to close, open towards the first set of the medicament containers Ma, Mb, Mc or open towards the second medicament container M0d′ (the emergency medicament). In this example, each of the first set of the medicament containers Ma, Mb, Mc is accommodated within an independent fluid-tight chamber 11a′″″″, 11b′″″″, 11c′″″″. In this example, only one of the fluid-tight chambers 11a″″″″ comprises the inlet connected to the fluid pressure power source 21. A one-way valve 101 is located between two fluid-tight chamber 11a″″″″, 11b″″″″, 11c″″″″. Therefore, only when the previous medicament container is empty, the pressure within the previous fluid-tight chamber can accumulate to open the one-way valve 101, thus the fluid from the fluid pressure power source 21 can flow into the next fluid-tight chamber.

In one example, each of the first set of the medicament container Ma, Mb, Mc used in the design as illustrated in FIG. 14 comprises a flexible bag. Additionally, each of the first set of the medicament container Ma, Mb, Mc used in the design as illustrated in FIG. 14 comprises a flexible tube as mentioned above. In this example, the second medicament container Md comprises a flexible bag. Additionally, the second set of the medicament container Md used in the design as illustrated in FIG. 14 comprises a flexible tube as mentioned above.

Alternatively, in another example, one example, each of the first set of the medicament container Ma, Mb, Mc used in the design as illustrated in FIG. 14 comprises a flexible bag. Additionally, each of the first set of the medicament container Ma, Mb, Mc used in the design as illustrated in FIG. 14 comprises a flexible tube as mentioned above. In this example, the second medicament container Md comprises a cartridge made of rigid material, e.g., glass or rigid plastic.

Additionally, the second set of the medicament container Md used in the design as illustrated in FIG. 14 comprises a flexible tube as mentioned above.

Furthermore, the examples as illustrated in FIGS. 13-14 are only the schematical views of multiple fluid-tight chambers. In one example, each of multiple fluid-tight chambers is accommodated in one independent cassette. Alternatively, all of multiple fluid-tight chambers are accommodated in one cassette. Alternatively, at least one of the multiple fluid-tight chambers are accommodated in one cassette, and the rest of the multiple fluid-tight chambers are accommodated in another cassette.

Furthermore, when the medicament delivery member 23; 23a, 23b, 23c, 23d is an injection needle or an insertion needle with a soft cannula, the processor can also detect a removal of the medicament delivery member 23; 23a, 23b, 23c, 23d when a predetermined pressure drop is detected as the resistant pressure of the target tissue will be less significant when the medicament delivery member 23; 23a, 23b, 23c, 23d is removed from the target tissue.

Furthermore, an intentional breakpoint can be arranged close to the medicament delivery member 23; 23a, 23b, 23c, 23d. For example, if the delivery tube of the body of the medicament container were to be pulled in a fashion that would normally dislodge the medicament delivery member 23; 23a, 23b, 23c, 23d from the patient, the delivery tube of the body of the medicament container would instead break at this joint separating the delivery tube of the body of the medicament container from the medicament delivery member 23; 23a, 23b, 23c, 23d to avoid any potential injury by the medicament delivery member 23; 23a, 23b, 23c, 23d.

This break would also have the effect of removing the pressure drop associated with the needle and subcutaneous tissue backpressure. Thus, for a medication delivered at a controlled flow rate, the upstream drive pressure would decrease.

Therefore, when removal of the medicament delivery member is detected, the processor can generate an indication on the user interface and/or send a warning signal to the remote device. The processor can also stop the fluid pressure power source 21 and open the release valve 115 to stop the medicament delivery operation.

Other events, such as the delivery tube containing air, or occlusion, can be detected with any pressure sensor and/or the flow sensor mentioned above, as shown in FIG. 36.

As mentioned above, the term ‘flow rate’, mentioned in the following examples mentioned the flow rate of the medicament leaves the medicament container.

Furthermore, in one example where the fluid pressure power source 21″; 21′″ is a pneumatic power source, the volume of the medicament within the medicament container may be determined using the Ideal Gas Law and its simplifications, e.g., Boyle's law and Charles's Law, as mentioned above in the summary section of the description. As mentioned above, for a commercial presentation of this device, it is unlikely, however, that the system uses pure ideal gases; use of ambient air is highly advantageous. To compensate for the inherent sensitivities to environmental variables and improve the pump performance in the presence of environmental uncertainty, the compensation block 21h″ as mentioned above can be used. The compensation block 21h″ comprising an atmospheric pressure sensor and/or an ambient temperature sensor such that the calculation can be calibrated based on the detection from the compensation block 21h″. Optionally, the compensation block can comprise humidity sensor to compensate for changes in air density. In a preferred example, the flow sensor can be integrated temperature sensor. It should be noted that the compensation block can be a unit with one or more sensors as mentioned above; alternatively, the compensation block is a control structure that is programmed in the one or more processors of the medicament delivery device, in this example, all sensors are arranged in the cassette and/or the fluid-tight chamber, and/or the pneumatic fluid pressure power source. In this example, all the sensors are connected to the one or more processors of the medicament delivery device.

Furthermore, in another example, the flow rate is not sensed directly. Rather, the system repeatedly calculates the void volume of the system. Provided the only way void volume can change is by liquid leaving the medicament container, the rate of change of the void volume equates to the flow rate.

To avoid noise observed in the liquid flow calculation when adjacent void volume measurements are used, it is possible to filter the measurements to obtain a cleaner signal. One such approach involves a buffer and linear regression. At each controller evaluation, the initial condition of the container carrier (calculated by the Ideal Gas Law and its simplifications) is added to a buffer and a linear regression is run on that buffer. The slope of the regression line is the flow rate.

The system allows for continuous control of flow rate. In one example, a target flow rate can be controlled by making continuous fluid delivery into the fluid-tight chamber while monitoring pressure, by removing the fluid flowed from the fluid pressure power source from the fluid-tight chamber while monitoring pressure, and/or by stopping flow abruptly (e.g., as in an emergency or during a systemic infusion reaction) by delivering the accumulated pressure or evacuating the fluid-tight chamber, reducing the pressure to ambient around the cassette. For example, the system can control the release valve to release the fluid to the ambient around the cassette based on a detection from one or more connected sensor(s). The system allows alteration of flow during medicament delivery operation, as may be needed during rate adjustment regimens common in oncology and provides for each cassette to have a desired flow rate that may be configured independently. Different medication cassettes or container carriers may be combined in a desired sequence, each cassette may have any desired fluid volume and may be delivered at a desired flow rate independently of viscosity, volume, or other medicament, patient, or system configuration (e.g., cannula gauge) parameters.

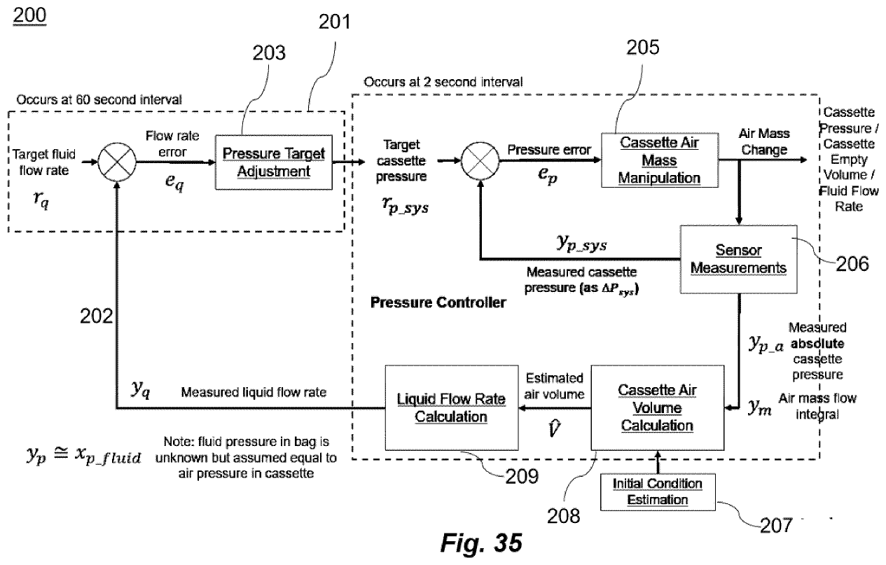

Furthermore, in one example where the fluid pressure power source is a pneumatic fluid pressure power source, the system can be control with a target maintenance mechanism, as shown in FIG. 35, the system (the operation of the fluid pressure power source when it connects to the fluid-tight chamber of the cassette) can be arranged with the target maintenance mechanism 200. In this example, the information about the initial condition of the cassette is measured by a measure module 207, 208, 209. The measure module comprises a flow rate calculation module 209 to calculate the medicament leaving rate from the fluid outlet the flexible bag, an air volume calculation module 208 to calculate the air volume within the fluid-tight chamber, and an initial information input 207. The flow rate information is input to the flow rate error detection module 201, and the pressure target can be adjusted via the adjustment module 203. The adjusted target pressure can be used to adjust the air mass in the fluid-tight chamber by the air mass module 205 together with the sensor module 206 to adjust the pressure level within the fluid-tight chamber. In this example, the target maintenance period simply adjusts the pressure target of the system proportionally to the measured flow rate error, as ideally there is a linear relationship between pressure and liquid flow rate. The flow rate error is interpreted as a ratio between target and measured liquid flow rate rather than a difference. The pressure target adjustment method may take many forms, but all inherently would be fed the flow error as an input for the response adjustment. Some controllers, such as Proportional Integral (PI) controller, Proportional-Integral-Derivative (PID) controller, or bang-bang controller can be used. In one example, the target maintenance mechanism can be designed with: the current system pressure and pressure target are evaluated by the PID function; the output of the PID function (in the form of a fluid pressure power source command) is compared to fluid pressure power source activity limitations, such as restricted power operation windows and on-time duration rules to ensure airflow sensing accuracy; and If permitted, the fluid pressure power source runs for the calculated amount of time. In a preferred example, time-based (rather than intensity-based) is used for the target maintenance mechanism, as intermittent, higher air flow rates are easier to sense accurately with a thermal mass flow sensor than continuous, low air flow rates. During each pressure control loop, the PID output is translated to fluid pressure power source on time (% of the control period). In one example where the fluid-tight chamber comprises the release valve, a release control mechanism can be used. The release control mechanism can be designed to compare the pressure target to the current system pressure. If the difference between those pressures is greater than a threshold and negative, and release control mechanism is activated. In a preferred example, when the release control mechanism is activated, the processor of the medicament delivery device is configured to pause the medicament delivery operation. In one example, the medicament delivery operation can be paused by a clamping the delivery tube. In this example, the medicament delivery device comprises a clamp that is electrically connected to the processor.

For example, the clamp is connected to a solenoid wire. Once the release control mechanism is activated, it starts of the vent (open the release valve), the system records the current void air volume and injected fluid mass, e.g., air mass, gas mass. Once the pressure has dropped below the target, the release control mechanism closes release valve, the system makes a new calculation of injected fluid mass, e.g., air mass, gas mass., and then provides the signal to the processor to continue the medicament delivery operation.

In a preferred example, the medicament delivery device can be controlled by the following method. The medicament delivery device comprises the fluid pressure power source being a pneumatic power source as mentioned in any of the above-mentioned examples. The medicament delivery device comprises the fluid-tight chamber as mentioned in any of the above-mentioned examples, the fluid-tight chamber contains a medicament container. The medicament container is the flexible bag having the fluid outlet. The flexible bag contains medicament. The method comprising the following steps in the following order:
receiving at least one of a pressure level measurement of fluid pressure within the fluid-tight chamber, and a flow rate measurement of the medicament leaving rate from the fluid outlet the flexible bag;
fetching information from a database with the received measurement; and
providing a signal based on the fetched information to make one or more electronic components of the medicament delivery device perform an action or stop the currently performing action of one or more electronic components of the medicament delivery device.

Preferably, both the pressure level measurement of fluid pressure within the fluid-tight chamber, and the flow rate measurement of the medicament leaving rate from the fluid outlet the flexible bag are received. In a preferred example, the method is performed by the processor of the medicament delivery device. In a preferred example, the fluid-tight chamber comprises a pressure sensor and a flow rate sensor. In a preferred example, the step of fetching information from a database with the received measurement comprises the following steps in the following order:
calculating, based on the received measurement with the Ideal Gas Law and its simplifications, a value;
comparing the calculated value and a predetermined value; and
generating a compared result.

In a preferred example, the calculation is carried out by the processor. Alternatively, or additionally, a communication unit of the medicament delivery device can send out the received measurement to a remote server and/or a personal computing device to be calculated. In this example, the processor is configured to receive the calculated value and/or the compared result (in the example where the comparison is also carried out in the remote server and/or the personal computing device).

It should be noted that the predetermined value is received from an information tag on the medicament container. Alternatively, or additionally, the predetermined value is entered from the user interface. Alternatively, or additionally, the predetermined value is downloaded from a remote server. Alternatively, or additionally, the predetermined value is saved in the processor and/or memory of the medicament delivery device. Furthermore, the predetermined value is about at least one of a volume of the medicament container, a volume of the medicament contained within the medicament container, a target flow rate of the medicament leaving rate from the fluid outlet the flexible bag, a target pressure level of fluid pressure within the fluid-tight chamber, a previously received flow rate of the medicament leaving rate from the fluid outlet the flexible bag, a previously received pressure level of fluid pressure within the fluid-tight chamber, previously calculated volume of the medicament contained within the medicament container.

Furthermore, in a preferred example, after the step of generating a compared result, the step of fetching information from a database with the received measurement comprises further comprises a step of: providing the fetched information by matching the compared result with information from the database. Alternatively, or additionally, after the step of generating a compared result, the step of fetching information from a database with the received measurement comprises further comprises a step of: providing the fetched information by providing the compared result.

The fetched information is about at least one of an actual filled volume of the medicament within the medicament container, a residual volume of the medicament within the medicament container after use, air in a delivery tube, a delivery member is away from a delivery site, and delivery occlusion (can be determined to be the ‘End of Dose’ event).

Furthermore, the action of the one or more electronic components of the medicament delivery device is configured to perform or stop by the provided signal is at least one of providing an indication to a user of the medicament delivery device, medicament delivery operation, sending data to a remote server, adjusting the pressure level of fluid pressure within the fluid-tight chamber, and adjusting the medicament leaving rate from the fluid outlet the flexible bag. Furthermore, the reusable body of the medicament delivery device, and/or the cassette housing of the cassette and/or the container carrier of the cassette and/or the fluid-tight chamber of the cassette, as mentioned in any example, may be provided with (i.e., molded in, molded with) a compound featuring persistently antimicrobial, antifungal, and/or antiviral properties. Alternatively, a compound featuring persistently antimicrobial, antifungal, and/or antiviral properties may be applied to the molded (i.e., finished) components through secondary processes (e.g., chemical vapor deposition), spraying, or dipping processes.

In a preferred example, the multi-direction valve, e.g., e.g., 2/2-way valve, 3/2-way valve, 5/2-way valve, as mentioned above can be a solenoid valve.

The inventive concept has mainly been described above with reference to a few examples. However, as is readily appreciated by a person skilled in the art, other embodiments than the ones disclosed above are equally possible within the scope of the inventive concept, as defined by the appended claims.

Some other aspects of the invention are disclosed in the clauses below.
1. A cassette (1; 1′; 1″) of a medicament delivery device (2; 2′; 2″), wherein the medicament delivery device comprises a reusable body (20; 20′) comprising a fluid pressure power source (21; 21′), the cassette (1; 1′; 1″) comprising:
   a container carrier (10; 10′; 10″; 10′″; 10a″, 10b″, 10c″, 10d″);
   a medicament container (M; Ma, Mb, Mc, Md; Ma′, Mb′, Mc′, Md′) with a body (MO; M0′; M0″; M0a, M0b; M0a, M0a′, M0a″, M0b, M0b′; M0b″) and a fluid outlet (M1; M1′); wherein the body (M0; M0′; M0″; M0a, M0b; M0a, M0a′, M0a″, M0b, M0b′; M0b″) comprises a flexible portion; wherein the medicament container (M; Ma, Mb, Mc, Md; Ma′, Mb′, Mc′, Md′) is at least partially arranged within the container carrier (10; 10′; 10″; 10′″; 10a″, 10b″, 10c″, 10d″);
   wherein the container carrier (10; 10′; 10″; 10′″; 10a″, 10b″, 10c″, 10d″) comprises a fluid-tight chamber (11; 11′; 11′″; 11a′″, 11b′″, 11c′″; 11a″″, 11b″″, 11c″″, 11d″″; 11′″″, 11″″″, 11a′″″″, 11b′″″″, 11e′″″″, 11d′″″″; 11a″″″″, 11b″″″″, 11c″″″″, 11d″″″″; 11a′″″″″, 10e′″″; 11a″″″″″, 11b″″″″″); wherein the fluid-tight chamber (11; 11′; 11″; 11a′″, 11b′″, 11c′″; 11a″″, 11b″″, 11c″″, 11d″″; 11′″″, 11″″″, 11a′″″″, 11b′″″″, 11c′″″″, 11d′″″″; 11a″″″″, 11b″″″″, 11c″″″″, 11d″″″″; 11a′″″″″, 10e′″″; 11a″″″″″, 11b″″″″″) comprises an inlet (110; 110′; 110″; 110′″; 110″″; 110a); wherein the inlet (110; 110′; 110″; 110′″; 110″″; 110a) is configured to be fluidly connected to an outlet of the fluid pressure power source of the reusable body of the medicament delivery device;
   wherein the fluid-tight chamber (11; 11′; 11″; 11a′″, 11b′″, 11c′″; 11a″″, 11b″″, 11c″″, 11d″″; 11′″″, 11″″″, 11a′″″″, 11b′″″″, 11c′″″″, 11d′″″″; 11a″″″″, 11b″″″″, 11e″″″″, 11d″″″″; 11a′″″″″, 10e′″″; 11a″″″″″, 11b″″″″″) is coupled to the flexible portion of the body (M0; M0′; M0″; M0a, M0b; M0a, M0a′, M0a″, M0b, M0b′; M0b″) of the medicament container (M; Ma, Mb, Mc, Md; Ma′, Mb′, Mc′, Md′) so that when the inlet (110; 110′; 110″; 110′″; 110″″; 110a) receives output fluid from the fluid pressure power source (21; 21′), the output fluid flows into the fluid-tight chamber (11; 11′; 11″; 11a′″, 11b′″, 11c′″; 11a″″, 11b″″, 11c″″, 11d″″; 11′″″, 11″″″, 11a′″″″, 11b′″″″, 11c′″″″, 11d′″″″; 11a″″″″, 11b″″″″, 11c″″″″, 11d″″″″; 11a″″″″″, 10e′″″; 11a″″″″″, 11b″″″″″) and at least part of the medicament contained within the medicament container (M; Ma, Mb, Mc, Md; Ma′, Mb′, Mc′, Md′) is pressed out under the pressure of the output fluid;
   and wherein the fluid outlet (M1; M1′) is configured to be connected to a medicament delivery member (23; 23a′, 23b′, 23c′, 23d′) of the medicament delivery device (2; 2′; 2″) when the cassette (1; 1′; 1″) is attached to the medicament delivery device (2; 2′; 2″).
2. The cassette according to clause 1, wherein the cassette comprises a cassette housing; and wherein the container carrier is arranged within the housing cassette housing.
3. The cassette according to clause 1 or 2, wherein the cassette is configured to be releasably attached to the reusable body of the medicament delivery device.
4. The cassette according to any one of the preceding clauses, wherein the body of the medicament container comprises a flexible bag and/or a flexible tube.
5. The cassette according to any one of the preceding clauses, wherein the body comprises a delivery tube.
6. The cassette according to a combination of clause 4 and clause 5, when the body of the medicament container comprises a flexible tube, wherein the delivery tube is the flexible tube of the body.
7. The cassette according to any one of the preceding clauses, wherein the flexible portion of the body is at least partially accommodated within the fluid-tight chamber.
8. The cassette according to a combination of clauses 6 and 7, wherein the fluid-tight chamber comprises a tube inlet and a tube outlet; and wherein the delivery tube is configured to be positioned between the tube inlet and the tube outlet.
9. The cassette according to clause 7 or 8, wherein the fluid-tight chamber comprises an outlet configured to be connected to a vacuum device, so that when the pressure within the fluid-tight chamber is reduced, medicament contained within the medicament container is sucked into the delivery tube.
10. The cassette according to clause 9, wherein the inlet of the fluid-tight chamber is the outlet of the fluid-tight chamber.
11. The cassette according to any one of clauses 1-6, wherein the container carrier comprises a container chamber configured to at least partially accommodate the medicament container; wherein the fluid-tight chamber is inflatable; and wherein the fluid-tight chamber is adjacent to the flexible portion of the body, so that the output fluid from the fluid pressure power source is configured to flow into the fluid-tight chamber and inflate the fluid-tight chamber to press on the medicament container.
12. The cassette according to any one of clauses 1-4, wherein the cassette comprises the container carrier with one fluid-tight chamber within the container carrier; wherein the container carrier comprises a connecting port configured to be releasably attached to either the outlet of the fluid pressure power source of the reusable body of the medicament delivery device or another connecting port of another container carrier such that the container carrier is configured to stacked on one another.
13. The cassette according to clauses 12, wherein the cassette comprises one container carrier comprising the connecting port configured to be releasably attached to either the outlet of the fluid pressure power source of the reusable body of the medicament delivery device or another connecting port of another container carrier of another cassette such that the multiple cassettes are configured to stacked on one another.
14. The cassette according to clauses 12 when dependent on clause 2 or clauses 3-4 when dependent on clauses 2, wherein the cassette comprises multiple container carrier stacked on one another within the cassette housing.
15. The cassette according to any one of clauses 1-4 or clauses 12-13, wherein the fluid-tight chamber is configured to receive the medicament container.
16. The cassette according to clause 14, wherein the fluid-tight chamber is formed by a container frame.
17. The cassette according to clause 15, wherein the fluid-tight chamber is formed by the container frame and an interior chamber of the container carrier.
18. The cassette according to clause 15, wherein the fluid-tight chamber is formed by the container frame and a cap configured to be attached to the container frame.
19. The cassette according to clause 17, wherein the cap comprises a tube set comprising a delivery tube operably connected to the fluid outlet of the medicament container within the fluid-tight chamber.
20. The cassette according to clause 18, wherein the tube set comprises a piercing member configured to penetrate the fluid outlet of the medicament container to establish fluid communication between the delivery tube and the medicament container.
21. The cassette according to any one of the preceding clauses, wherein the inlet of the fluid-tight chamber comprises a valve.
22. The cassette according to clause 21, wherein the valve of the inlet of the fluid-tight chamber is a one-way valve.
23. The cassette according to any one of the preceding clauses, wherein the cassette comprises at least two fluid-tight chambers; and wherein the two fluid-tight chambers accommodate at least two medicament containers respectively.
24. The cassette according to clause 23, wherein a one-way valve is arranged between the two fluid-tight chambers.
25. The cassette according to clause 24, wherein only one fluid-tight chamber of one of the at least two fluid-tight chambers comprises the inlet that is configured to be fluidly connected to the fluid pressure power source.
26. The cassette according to clause 25, when dependent on clause 21 or 22, wherein the one-way valve between the two fluid-tight chambers is configured to open when a first fluid pressure threshold is reached; wherein the valve of the inlet of the fluid-tight chamber that is configured to be fluidly connected to the fluid pressure power source is configured to open when a second fluid pressure threshold is reached; and wherein the first fluid pressure threshold is equal to or greater than the second fluid pressure threshold.
27. The cassette according to any one of the preceding clauses, wherein the fluid-tight chamber comprises a release valve configured to release fluid that flows into the fluid-tight chamber so that the fluid can flow out of the fluid-tight chamber through the release valve.
28. The cassette according to clause 18 when dependent on clause 21 or 22, wherein the valve of the inlet of the fluid-tight chamber that is configured to be fluidly connected to the fluid pressure power source is configured to open when a second fluid pressure threshold is reached; wherein the release valve configured to release the fluid that flows into the fluid-tight chamber out of the fluid-tight chamber when the fluid pressure reaches a predetermined threshold; and wherein the predetermined threshold is greater than the second fluid pressure threshold.
29. The cassette according to clause 28 when dependent on clause 26, wherein the predetermined threshold is greater than the first fluid pressure threshold.
30. The cassette according to any one of the preceding clauses, wherein the fluid-tight chamber of the container carrier is at least partially made of rigid material.
31. The cassette according to any one of the preceding clauses, wherein the fluid-tight chamber comprises a pressure sensor, and/or wherein the fluid-tight chamber is coupled to a fluid-tight measure chamber; wherein the fluid-tight measure chamber is configured to be connected to the fluid pressure power source and is configured to be at the same fluid pressure level as the fluid-tight chamber; wherein the fluid-tight measure chamber comprises a piston; and wherein the piston is configured to be operably connected to a position sensor configured to sense the position of the piston within the fluid-tight measure chamber, and/or wherein the container carrier comprises a position sensor configured to detect the position of the medicament container.
32. The cassette according to any one of the preceding clauses, wherein the container carrier partially comprises the fluid-tight chamber that is configured to be formed by a combination of the container carrier and the reusable body of the medicament delivery device when the cassette is attached to the reusable body of the medicament delivery device.
33. A medicament delivery device comprising the cassette according to any one of clauses 1-32, wherein the medicament delivery device comprises a reusable body and a changeable medicament delivery member; wherein the reusable body of the medicament delivery device comprises the fluid pressure power source connected to the inlet of the fluid-tight chamber of the container carrier; and wherein the fluid outlet is operably connected to the medicament delivery member.
34. The medicament delivery device according to clause 33, wherein the fluid-tight chamber is formed by the container carrier of the cassette and the reusable body when the cassette is attached to the reusable body.
35. The medicament delivery device according to clause 33 or 34, wherein the medicament delivery device comprises a processor electrically connected to the fluid pressure power source, an electrical power source connected to the processor; wherein the processor, and the electrical power source are accommodated within the reusable body.
36. The medicament delivery device according to clause 35, wherein a transmission tube is arranged between the fluid pressure power source and the inlet of the fluid-tight chamber; and wherein the fluid pressure power source is fluidly connected to the inlet of the fluid-tight chamber via the transmission tube.
37. The medicament delivery device according to clause 35, the fluid-tight chamber is adjacent to the fluid pressure power source; and wherein the fluid pressure power source is fluidly connected to the inlet of the fluid-tight chamber directly.
38. The medicament delivery device according to any one of clauses 35-37, wherein the medicament delivery device comprises a user interface attached to the reusable body; wherein the user interface is electrically connected to the processor; wherein the user interface is a button protruding from an outer surface of the reusable body; or wherein the user interface is a touch panel arranged on an outer surface of the reusable body.
39. The medicament delivery device according to any one of clauses 35-38, when dependent on clause 31, wherein the pressure sensor and/or the position sensor, or the fluid-tight measure chamber and/or the position sensor, of the container carrier is electrically connected to the processor.
40. The medicament delivery device according to clause 39, wherein the processor is configured to control the fluid pressure power source to output fluid into the fluid-tight chamber according to a signal from the pressure sensor and/or the position sensor, or the fluid-tight measure chamber and/or the position sensor, of the container carrier.
41. The medicament delivery device according to any one of clauses 35-40, when dependent on clause 14, wherein the fluid pressure power source is connected to at least two fluid-tight chambers; and wherein the processor is configured to control the fluid pressure power source to selectively output fluid into at least one the fluid-tight chambers.
42. The medicament delivery device according to clause 41, when dependent on clause 39, wherein the processor is configured to control the fluid pressure power source to selectively output fluid into at least one the fluid-tight chambers according to a signal from the pressure sensor and/or the position sensor or the fluid-tight measure chamber and/or the position sensor of the container carrier.
43. The medicament delivery device according to clause 39 or clause 38, wherein the processor is configured to control the fluid pressure power source to output a certain amount of fluid; wherein the certain amount is predetermined or is dependent on a signal from the pressure sensor and/or the position sensor or the fluid-tight measure chamber and/or the position sensor of the container carrier; thereby only certain amount of medicament contained within the medicament container can be pressed out of the fluid outlet.
44. The medicament delivery device according to any one of clauses 33-43, wherein the fluid pressure power source is a pneumatic power source.
45. The medicament delivery device according to clause 44, wherein the pneumatic power source comprises a piezo pump configured to cause fluid to output from the pneumatic power source.
46. The medicament delivery device according to clause 44 or 45, wherein the fluid outputted from the pneumatic power source is gas.
47. The medicament delivery device according to any one of clauses 33-34, wherein the medicament delivery device is an infusion device.
48. The medicament delivery device according to any one of clauses 33-47, wherein the fluid pressure power source comprises a fluid pump having an inlet fluidly connected to the environment and an inlet filter connected to the inlet of the fluid pump comprises an inlet filter connected to the fluid pump.
49. The medicament delivery device according to any one of clauses 33-48, wherein the fluid pressure power source comprises a multi-directions valve; wherein the outlet port of each multi-directions valve defines the outlet of the fluid pressure power source.
50. The medicament delivery device according to any one of clauses 33-49, comprising a multi-directions valve connected to the outlet of the fluid pressure power source.
51. The medicament delivery device according to any one of clauses 33-50, comprising a tube set having a delivery tube and a piercing member; wherein the piercing member is configured to establish a fluid connection between the delivery tube and the medicament container.
52. A method for controlling a medicament delivery device having a fluid pressure power source being a pneumatic power source and a fluid-tight chamber containing a medicament container; wherein an inlet of the fluid-tight chamber is connected to the outlet of the fluid pressure power source; wherein the medicament container is a flexible bag having a fluid outlet; wherein the flexible bag contains medicament, the method comprising the following steps in the following order:
   receiving at least one of a pressure level measurement of fluid pressure within the fluid-tight chamber, and a flow rate measurement of the medicament leaving rate from the fluid outlet the flexible bag;
   fetching information from a database with the received measurement; and
   providing a signal based on the fetched information to make one or more electronic components of the medicament delivery device perform an action or stop the currently performing action of one or more electronic components of the medicament delivery device.
53. The method according to clause 52, wherein the step of receiving at least one of a pressure level measurement of fluid pressure within the fluid-tight chamber, and a flow rate measurement of the medicament leaving rate from the fluid outlet the flexible bag comprises a step of:
   receiving a pressure level measurement of fluid pressure within the fluid-tight chamber, and a flow rate measurement of the medicament leaving rate from the fluid outlet the flexible bag.
54. The method according to clause 52 or 53, wherein the step of fetching information from a database with the received measurement comprises the following steps in the following order:
   calculating, based on the received measurement with the Ideal Gas Law and its simplifications, a value;
   comparing the calculated value and a predetermined value; and
   generating a compared result.
55. The method according to clause 54, wherein after the step of generating a compared result, the step of fetching information from a database with the received measurement comprises further comprises a step of:
   providing the fetched information by matching the compared result with information from the database.
56. The method according to clause 54 or clause 55, wherein after the step of generating a compared result, the step of fetching information from a database with the received measurement comprises further comprises a step of:
   providing the fetched information by providing the compared result.
57. The method according to any one of clauses 56 or 57, wherein the fetched information is about at least one of an actual filled volume of the medicament within the medicament container, a residual volume of the medicament within the medicament container after use, air in a delivery tube, a delivery member is away from a delivery site, and delivery occlusion.
58. The method according to any one of clauses 54-57, wherein the predetermined value is about at least one of a volume of the medicament container, a volume of the medicament contained within the medicament container, a target flow rate of the medicament leaving rate from the fluid outlet the flexible bag, a target pressure level of fluid pressure within the fluid-tight chamber, a previously received flow rate of the medicament leaving rate from the fluid outlet the flexible bag, a previously received pressure level of fluid pressure within the fluid-tight chamber, previously calculated volume of the medicament contained within the medicament container.
59. The method according to clause 58, wherein the predetermined value is received from an information tag on the medicament container.
60. The method according to any one of clauses 52-59, wherein the action of the one or more electronic components of the medicament delivery device is configured to perform or stop by the provided signal is at least one of providing an indication to a user of the medicament delivery device, medicament delivery operation, sending data to a remote server, adjusting the pressure level of fluid pressure within the fluid-tight chamber, and adjusting the medicament leaving rate from the fluid outlet the flexible bag.
61. The medicament delivery device according to clause 35 or any one of clauses 36-51 when dependent on clause 35, wherein the processor is configured to perform the method according to any one of clauses 48-56.
62. A medicament delivery device comprising the cassette according to any one of clauses 1-32, wherein the medicament delivery device comprises a reusable body and a changeable medicament delivery member; wherein the reusable body of the medicament delivery device comprises the fluid pressure power source connected to the inlet of the fluid-tight chamber of the container carrier; wherein the fluid outlet is operably connected to the medicament delivery member; wherein the medicament delivery device comprises a processor configured to perform the method according to any one of clauses 48-56.
63. The medicament delivery device according to clause 62, wherein the fluid-tight chamber of the cassette is operably connected to a pressure sensor configured to measure a pressure level measurement of fluid pressure within the fluid-tight chamber and/or a flow rate sensor configured to measure the medicament leaving rate from the fluid outlet the flexible bag.
64. The medicament delivery device according to clause 63, wherein the cassette is operably connected to the pressure sensor and the flow rate sensor.
65. The medicament delivery device according to clause 64, wherein the cassette comprises the pressure sensor and the flow rate sensor.
66. The medicament delivery device according to any one of clauses 62-65, wherein the reusable body of the medicament delivery device comprises the processor.
67. The medicament delivery device according to clause 66, wherein the processor electrically connected to the fluid pressure power source.
68. The medicament delivery device according to a combination of clause 67 and any one of clauses 66-67, wherein the processor electrically connected to the pressure sensor and the flow rate sensor when the cassette is attached to the reusable body of the medicament delivery device.
69. The medicament delivery device according to any one of clauses 62-68, wherein the reusable body of the medicament delivery device comprises a communication unit configured to read an information tag on the medicament cassette when the cassette is attached to the reusable body of the medicament delivery device.
70. The medicament delivery device according to a combination of clause 68 and clause 69, wherein the communication unit is electrically connected to the processor.
71. The medicament delivery device according to any one of clauses 69 or 70, wherein the communication unit is an RFID/NFC reader and/or RFID/NFC writer.
72. The medicament delivery device according to any one of clauses 62-71, wherein the pneumatic power source comprises a piezo pump configured to cause fluid to output from the pneumatic power source.
73. The medicament delivery device according to clause 72, wherein the fluid outputted from the pneumatic power source is gas.
74. The medicament delivery device according to any one of clauses 62-73, wherein the medicament delivery device is an infusion device.
75. The medicament delivery device according to any one of clauses 62-74, wherein the fluid pressure power source comprises a fluid pump having an inlet fluidly connected to the environment and an inlet filter connected to the inlet of the fluid pump comprises an inlet filter connected to the fluid pump.
76. The medicament delivery device according to any one of clauses 62-75, wherein the fluid pressure power source comprises a multi-directions valve; wherein the outlet port of each multi-directions valve defines the outlet of the fluid pressure power source.
77. The medicament delivery device according to any one of clauses 62-76, comprising a multi-directions valve connected to the outlet of the fluid pressure power source.
78. The medicament delivery device according to any one of clauses 62-77, comprising a tube set having a delivery tube and a piercing member; wherein the piercing member is configured to establish a fluid connection between the delivery tube and the medicament container.

1-15. (canceled)

16. A cassette configured for use with a medicament delivery device, wherein the medicament delivery device comprises a reusable body comprising a fluid pressure power source, the cassette comprising:
a container carrier; and
a medicament container with a body and a fluid outlet, wherein the body comprises a flexible portion, and wherein the medicament container is at least partially arranged within the container carrier,
wherein the container carrier comprises a fluid-tight chamber, wherein the fluid-tight chamber comprises an inlet, wherein the inlet is configured to be fluidly connected to an outlet of the fluid pressure power source of the reusable body of the medicament delivery device,
wherein the fluid-tight chamber is coupled to the flexible portion of the body of the medicament container so that when the inlet receives output fluid from the fluid pressure power source, the output fluid flows into the fluid-tight chamber and at least part of the medicament contained within the medicament container is pressed out under the pressure of the output fluid,
and wherein the fluid outlet is configured to be connected to a medicament delivery member of the medicament delivery device when the cassette is attached to the medicament delivery device.

17. The cassette of claim 16, wherein the body of the medicament container comprises a flexible bag received within the fluid-tight chamber.

18. The cassette of claim 16, wherein the cassette comprises the container carrier with one fluid-tight chamber within the container carrier, and wherein the container carrier comprises a connecting port configured to be releasably attached to either the outlet of the fluid pressure power source of the reusable body of the medicament delivery device or another connecting port of another container carrier such that the container carrier is configured to stacked on one another.

19. The cassette of claim 18, wherein the cassette comprises one container carrier comprising the connecting port configured to be releasably attached to either the outlet of the fluid pressure power source of the reusable body of the medicament delivery device or another connecting port of another container carrier of another cassette such that the multiple cassettes are configured to stacked on one another.

20. The cassette of claim 18, wherein the cassette comprises a cassette housing configured to be releasably attached to the reusable body of the medicament delivery device, and wherein the cassette comprises multiple container carrier stacked on one another within the cassette housing.

21. The cassette of claim 16, wherein the fluid-tight chamber is formed by a container frame.

22. The cassette of claim 21, wherein the fluid-tight chamber is formed by the container frame and an interior chamber of the container carrier.

23. The cassette of claim 16, wherein the fluid-tight chamber is formed by the container frame and a cap configured to be attached to the container frame.

24. The cassette of claim 23, wherein the cap comprises a tube set comprising a delivery tube operably connected to the fluid outlet of the medicament container within the fluid-tight chamber.

25. The cassette of claim 24, wherein the tube set comprises a piercing member configured to penetrate the fluid outlet of the medicament container to establish fluid communication between the delivery tube and the medicament container.

26. The cassette of claim 16, wherein the fluid-tight chamber comprises a release valve configured to release fluid that flows into the fluid-tight chamber so that the fluid can flow out of the fluid-tight chamber through the release valve.

27. A medicament delivery device comprising the cassette of claim 16, wherein the medicament delivery device comprises a reusable body and a changeable medicament delivery member, wherein the reusable body of the medicament delivery device comprises the fluid pressure power source connected to the inlet of the fluid-tight chamber of the container carrier, and wherein the fluid outlet is operably connected to the medicament delivery member.

28. The medicament delivery device of claim 27, wherein the fluid pressure power source comprises a multi-directions valve, and wherein the outlet port of each multi-directions valve defines the outlet of the fluid pressure power source.

29. The medicament delivery device of claim 27, comprising a multi-directions valve connected to the outlet of the fluid pressure power source.

30. The medicament delivery device of claim 27, wherein the fluid pump is a piezo pump.
